(19) United States
(12) Patent Application Publication
(10) Pub. No.: US 2024/0315700 A1
(43) Pub. Date: Sep. 26, 2024

(54) Globular Mesh Compressed into Multilayer Bowl-Shaped Aneurysm Neck Bridge

(71) Applicant: Robert A. Connor, Wyoming, MN (US)

(72) Inventor: Robert A. Connor, Wyoming, MN (US)

(73) Assignee: Aneuclose LLC, Ham Lake, MN (US)

(21) Appl. No.: 18/674,996

(22) Filed: May 27, 2024

Related U.S. Application Data
(63) Continuation-in-part of application No. 18/613,053, filed on Mar. 21, 2024.
(63) Continuation-in-part of application No. 18/519,055, filed on Nov. 26, 2023, now Pat. No. 12,004,750.
Application No. 18/613,053 is a continuation-in-part of application No. 18/519,055, filed on Nov. 26, 2023, now Pat. No. 12,004,750.
Application No. 18/519,055 is a continuation-in-part of application No. 18/135,153, filed on Apr. 15, 2023, now abandoned.
Application No. 18/519,055 is a continuation-in-part of application No. 18/374,602, filed on Sep. 28, 2023, now Pat. No. 11,944,316.
Application No. 18/374,602 is a continuation-in-part of application No. 18/135,153, filed on Apr. 15, 2023, now abandoned.
Application No. 18/135,153 is a continuation-in-part of application No. 17/970,510, filed on Oct. 20, 2022.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 18/374,602 is a continuation-in-part of application No. 18/135,153, filed on Apr. 15, 2023, now abandoned.
Application No. 18/135,153 is a continuation-in-part of application No. 17/970,510, filed on Oct. 20, 2022.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 18/135,153 is a continuation-in-part of application No. 17/970,510, filed on Oct. 20, 2022.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 17/829,313 is a continuation-in-part of application No. 17/476,845, filed on Sep. 16, 2021, now Pat. No. 11,583,289.
Application No. 17/965,502 is a continuation-in-part of application No. 17/829,313, filed on May 31, 2022, now abandoned.
Application No. 17/829,313 is a continuation-in-part of application No. 17/476,845, filed on Sep. 16, 2021, now Pat. No. 11,583,289.
Application No. 17/829,313 is a continuation-in-part of application No. 17/485,390, filed on Sep. 25, 2021, now Pat. No. 11,471,164.
Application No. 17/485,390 is a continuation-in-part of application No. 17/476,845, filed on Sep. 16, 2021, now Pat. No. 11,583,289.
Application No. 17/476,845 is a continuation-in-part of application No. 17/472,674, filed on Sep. 12, 2021, now abandoned.
Application No. 17/472,674 is a continuation-in-part of application No. 17/467,680, filed on Sep. 7, 2021, now abandoned.
Application No. 17/467,680 is a continuation-in-part of application No. 17/466,497, filed on Sep. 3, 2021, now Pat. No. 11,357,511.
Application No. 17/466,497 is a continuation-in-part of application No. 17/353,652, filed on Jun. 21, 2021, now abandoned.
Application No. 17/353,652 is a continuation-in-part of application No. 17/220,002, filed on Apr. 1, 2021, now Pat. No. 11,464,518.
Application No. 17/220,002 is a continuation-in-part of application No. 17/214,827, filed on Mar. 27, 2021, now Pat. No. 11,484,322.
Application No. 17/214,827 is a continuation-in-part of application No. 17/211,446, filed on Mar. 24, 2021, now abandoned.
Application No. 17/220,002 is a continuation-in-part of application No. 17/214,827, filed on Mar. 27, 2021, now Pat. No. 11,484,322.
Application No. 17/214,827 is a continuation-in-part of application No. 17/211,446, filed on Mar. 24, 2021, now abandoned.
Application No. 17/211,446 is a continuation-in-part of application No. 16/693,267, filed on Nov. 23, 2019, now abandoned.
Application No. 16/693,267 is a continuation-in-part of application No. 16/660,929, filed on Oct. 23, 2019, now Pat. No. 11,471,163.
Application No. 16/693,267 is a continuation-in-part of application No. 16/660,929, filed on Oct. 23, 2019, now Pat. No. 11,471,163.
Application No. 16/660,929 is a continuation-in-part of application No. 16/541,241, filed on Aug. 15, 2019, now abandoned.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 16/541,241, filed on Aug. 15, 2019, now abandoned.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 15/865,822, filed on Jan. 9, 2018, now Pat. No. 10,716,573.
Application No. 15/865,822 is a continuation-in-part of application No. 15/081,909, filed on Mar. 27, 2016, now abandoned.
Application No. 15/081,909 is a continuation-in-part of application No. 14/526,600, filed on Oct. 29, 2014, now abandoned.
Application No. 14/526,600 is a continuation-in-part of application No. 15/080,915, filed on Mar. 25, 2016, now Pat. No. 10,028,747.
Application No. 15/080,915 is a continuation-in-part of application No. 14/526,600, filed on Oct. 29, 2014, now abandoned.
Application No. 15/081,909 is a continuation-in-part of application No. 14/526,600, filed on Oct. 29, 2014, now abandoned.
Application No. 15/080,915 is a continuation-in-part of application No. 14/526,600, filed on Oct. 29, 2014, now abandoned.
Application No. 14/526,600 is a continuation-in-part of application No. 12/989,048, filed on Oct. 21, 2010, now Pat. No. 8,974,487.
(60) Provisional application No. 63/119,774, filed on Dec. 1, 2020.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/720,173, filed on Aug. 21, 2018.
(60) Provisional application No. 62/589,754, filed on Nov. 22, 2017.
(60) Provisional application No. 62/472,519, filed on Mar. 16, 2017.
(60) Provisional application No. 62/589,754, filed on Nov. 22, 2017.
(60) Provisional application No. 62/472,519, filed on Mar. 16, 2017.
(60) Provisional application No. 62/444,860, filed on Jan. 11, 2017.
(60) Provisional application No. 61/897,245, filed on Oct. 30, 2013.
(60) Provisional application No. 61/126,047, filed on May 1, 2008.
(60) Provisional application No. 61/126,027, filed on May 1, 2008.

(51) Int. Cl. A61B 17/12

(52) U.S. Cl. CPC A61B 17/12113; A61B 17/12172; A61B 17/12186; A61B 2017/1205

(57) ABSTRACT

This invention is an intrasaccular aneurysm occlusion device. A proximal portion of the device has a single layer in a first configuration and multiple layers in a second configuration. The device can have a single-layer globular shape in the first configuration and a double-layer bowl shape in the second configuration. The device can be collapsed, compressed, inverted, and/or folded from the first configuration to the second configuration by the accumulation of embolic material in the aneurysm sac, wherein the embolic material is inserted through an opening in the device.

### CROSS-REFERENCE TO RELATED APPLICATIONS

This application is a continuation-in-part of U.S. patent application Ser. No. 18/519,055 filed on 2023 Nov. 26 and a continuation-in-part of U.S. patent application Ser. No. 18/135,153 filed on 2023 Apr. 15.

This application is a continuation-in-part of U.S. patent application Ser. No. 18/613,053 filed on 2024 Mar. 21 and a continuation-in-part of U.S. patent application Ser. No. 18/519,055 filed on 2023 Nov. 26.

U.S. patent application Ser. No. 18/613,053 was a continuation-in-part of U.S. patent application Ser. No. 18/519,055 filed on 2023 Nov. 26 and a continuation-in-part of U.S. patent application Ser. No. 18/135,153 filed on 2023 Apr. 15.

U.S. patent application Ser. No. 18/519,055 was a continuation-in-part of U.S. patent application Ser. No. 18/374,602 filed on 2023 Sep. 28, a continuation-in-part of U.S. patent application Ser. No. 18/135,153 filed on 2023 Apr. 15, a continuation-in-part of U.S. patent application Ser. No. 17/970,510 filed on 2022 Oct. 20, a continuation-in-part of U.S. patent application Ser. No. 17/965,502 filed on 2022 Oct. 13, and a continuation-in-part of U.S. patent application Ser. No. 17/829,313 filed on 2022 May 31.

U.S. patent application Ser. No. 18/374,602 was a continuation-in-part of U.S. patent application Ser. No. 18/135,153 filed on 2023 Apr. 15, a continuation-in-part of U.S. patent application Ser. No. 17/970,510 filed on 2022 Oct. 20, a continuation-in-part of U.S. patent application Ser. No. 17/965,502 filed on 2022 Oct. 13, and a continuation-in-part of U.S. patent application Ser. No. 17/829,313 filed on 2022 May 31.

U.S. patent application Ser. No. 18/135,153 was a continuation-in-part of U.S. patent application Ser. No. 17/970,510 filed on 2022 Oct. 20, a continuation-in-part of U.S. patent application Ser. No. 17/965,502 filed on 2022 Oct. 13, and a continuation-in-part of U.S. patent application Ser. No. 17/829,313 filed on 2022 May 31.

U.S. patent application Ser. No. 17/970,510 was a continuation-in-part of U.S. patent application Ser. No. 17/965,502 filed on 2022 Oct. 13, a continuation-in-part of U.S. patent application Ser. No. 17/829,313 filed on 2022 May 31, and a continuation-in-part of U.S. patent application Ser. No. 17/476,845 filed on 2021 Sep. 16.

U.S. patent application Ser. No. 17/829,313 was a continuation-in-part of U.S. patent application Ser. No. 17/485,390 filed on 2021 Sep. 25, was a continuation-in-part of U.S. patent application Ser. No. 17/476,845 filed on 2021 Sep. 16, was a continuation-in-part of U.S. patent application Ser. No. 17/472,674 filed on 2021 Sep. 12, was a continuation-in-part of U.S. patent application Ser. No. 17/467,680 filed on 2021 Sep. 7, was a continuation-in-part of U.S. patent application Ser. No. 17/466,497 filed on 2021 Sep. 3, was a continuation-in-part of U.S. patent application Ser. No. 17/353,652 filed on 2021 Jun. 21, was a continuation-in-part of U.S. patent application Ser. No. 17/220,002 filed on 2021 Apr. 1, was a continuation-in-part of U.S. patent application Ser. No. 17/214,827 filed on 2021 Mar. 27, was a continuation-in-part of U.S. patent application Ser. No. 17/211,446 filed on 2021 Mar. 24, was a continuation-in-part of U.S. patent application Ser. No. 16/693,267 filed on 2019 Nov. 23, and was a continuation-in-part of U.S. patent application Ser. No. 16/660,929 filed on 2019 Oct. 23.

U.S. patent application Ser. No. 17/220,002 was a continuation-in-part of U.S. patent application Ser. No. 17/214,827 filed on 2021 Mar. 27. U.S. patent application Ser. No. 17/220,002 was a continuation-in-part of U.S. patent application Ser. No. 17/211,446 filed on 2021 Mar. 24. U.S. patent application Ser. No. 17/220,002 claimed the priority benefit of U.S. provisional patent application 63/119,774 filed on 2020 Dec. 1. U.S. patent application Ser. No. 17/220,002 was a continuation-in-part of U.S. patent application Ser. No. 16/693,267 filed on 2019 Nov. 23. U.S. patent application Ser. No. 17/220,002 was a continuation-in-part of U.S. patent application Ser. No. 16/660,929 filed on 2019 Oct. 23.

U.S. patent application Ser. No. 16/693,267 was a continuation-in-part of U.S. patent application Ser. No. 16/660,929 filed on 2019 Oct. 23. U.S. patent application Ser. No. 16/693,267 claimed the priority benefit of U.S. provisional patent application 62/794,609 filed on 2019 Jan. 19. U.S. patent application Ser. No. 16/693,267 claimed the priority benefit of U.S. provisional patent application 62/794,607 filed on 2019 Jan. 19. U.S. patent application Ser. No. 16/693,267 was a continuation-in-part of U.S. patent application Ser. No. 16/541,241 filed on 2019 Aug. 15. U.S. patent application Ser. No. 16/693,267 was a continuation-in-part of U.S. patent application Ser. No. 15/865,822 filed on 2018 Jan. 9 which issued as U.S. Pat. No. 10,716,573 on 2020 Jul. 21. U.S. patent application Ser. No. 16/693,267 was a continuation-in-part of U.S. patent application Ser. No. 15/861,482 filed on 2018 Jan. 3.

U.S. patent application Ser. No. 16/660,929 claimed the priority benefit of U.S. provisional patent application 62/794,609 filed on 2019 Jan. 19. U.S. patent application Ser. No. 16/660,929 claimed the priority benefit of U.S. provisional patent application 62/794,607 filed on 2019 Jan. 19. U.S. patent application Ser. No. 16/660,929 was a continuation-in-part of U.S. patent application Ser. No. 16/541,241 filed on 2019 Aug. 15. U.S. patent application Ser. No. 16/660,929 was a continuation-in-part of U.S. patent application Ser. No. 15/865,822 filed on 2018 Jan. 9 which issued as U.S. Pat. No. 10,716,573 on 2020 Jul. 21. U.S. patent application Ser. No. 16/660,929 was a continuation-in-part of U.S. patent application Ser. No. 15/861,482 filed on 2018 Jan. 3.

U.S. patent application Ser. No. 16/541,241 claimed the priority benefit of U.S. provisional patent application 62/794,609 filed on 2019 Jan. 19. U.S. patent application Ser. No. 16/541,241 claimed the priority benefit of U.S. provisional patent application 62/794,607 filed on 2019 Jan. 19. U.S. patent application Ser. No. 16/541,241 claimed the priority benefit of U.S. provisional patent application 62/720,173 filed on 2018 Aug. 21. U.S. patent application Ser. No. 16/541,241 was a continuation-in-part of U.S. patent application Ser. No. 15/865,822 filed on 2018 Jan. 9 which issued as U.S. Pat. No. 10,716,573 on 2020 Jul. 21

U.S. patent application Ser. No. 15/865,822 claimed the priority benefit of U.S. provisional patent application 62/589,754 filed on 2017 Nov. 22. U.S. patent application Ser. No. 15/865,822 claimed the priority benefit of U.S. provisional patent application 62/472,519 filed on 2017 Mar. 16. U.S. patent application Ser. No. 15/865,822 was a continuation-in-part of U.S. patent application Ser. No. 15/081,909 filed on 2016 Mar. 27. U.S. patent application Ser. No. 15/865,822 was a continuation-in-part of U.S. patent application Ser. No. 14/526,600 filed on 2014 Oct. 29.

U.S. patent application Ser. No. 15/861,482 claimed the priority benefit of U.S. provisional patent application 62/589,754 filed on 2017 Nov. 22. U.S. patent application Ser. No. 15/861,482 claimed the priority benefit of U.S. provisional patent application 62/472,519 filed on 2017 Mar. 16. U.S. patent application Ser. No. 15/861,482 claimed the priority benefit of U.S. provisional patent application 62/444,860 filed on 2017 Jan. 11. U.S. patent application Ser. No. 15/861,482 was a continuation-in-part of U.S. patent application Ser. No. 15/080,915 filed on 2016 Mar. 25 which issued as U.S. Pat. No. 10,028,747 on 2018 Jul. 24. U.S. patent application Ser. No. 15/861,482 was a continuation-in-part of U.S. patent application Ser. No. 14/526,600 filed on 2014 Oct. 29.

U.S. patent application Ser. No. 15/081,909 was a continuation-in-part of U.S. patent application Ser. No. 14/526,600 filed on 2014 Oct. 29. U.S. patent application Ser. No. 15/080,915 was a continuation-in-part of U.S. patent application Ser. No. 14/526,600 filed on 2014 Oct. 29. U.S. patent application Ser. No. 14/526,600 claimed the priority benefit of U.S. provisional patent application 61/897,245 filed on 2013 Oct. 30. U.S. patent application Ser. No. 14/526,600 was a continuation-in-part of U.S. patent application Ser. No. 12/989,048 filed on 2010 Oct. 21 which issued as U.S. Pat. No. 8,974,487 on 2015 Mar. 10. U.S. patent application Ser. No. 12/989,048 claimed the priority benefit of U.S. provisional patent application 61/126,047 filed on 20 Aug. 5, 2001. U.S. patent application Ser. No. 12/989,048 claimed the priority benefit of U.S. provisional patent application 61/126,027 filed on 20 Aug. 5, 2001.

The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to aneurysm occlusion devices and methods.

### INTRODUCTION

An aneurysm is an abnormal bulging of a blood vessel wall. The vessel from which the aneurysm protrudes is the parent vessel. Saccular aneurysms look like a sac protruding out from the parent vessel. Saccular aneurysms have a neck and can be prone to rupture. Fusiform aneurysms are a form of aneurysm in which a blood vessel is expanded circumferentially in all directions. Fusiform aneurysms generally do not have a neck and are less prone to rupturing than saccular aneurysms. As an aneurysm grows larger, its walls generally become thinner and weaker. This decrease in wall integrity, particularly for saccular aneurysms, increases the risk of the aneurysm rupturing and hemorrhaging blood into the surrounding tissue, with serious and potentially fatal health outcomes.

Cerebral aneurysms, also called brain aneurysms or intracranial aneurysms, are aneurysms that occur in the intercerebral arteries that supply blood to the brain. The majority of cerebral aneurysms form at the junction of arteries at the base of the brain that is known as the Circle of Willis where arteries come together and from which these arteries send branches to different areas of the brain. Although identification of intact aneurysms is increasing due to increased use of outpatient imaging such as outpatient MRI scanning, many cerebral aneurysms still remain undetected unless they rupture. If they do rupture, they often cause stroke, disability, and/or death. The prevalence of cerebral aneurysms is generally estimated to be in the range of 1%-5% of the general population or approximately 3-15 million people in the U.S. alone. Approximately 30,000 people per year suffer a ruptured cerebral aneurysm in the U.S. alone. Approximately one-third to one-half of people who suffer a ruptured cerebral aneurysm die within one month of the rupture. Sadly, even among those who survive, approximately one-half suffer significant and permanent deterioration of brain function. Better alternatives for cerebral aneurysm treatment are needed.

### Review of the Relevant Art

U.S. patent application 20210128161 (Nageswaran et al., May 6, 2021, “Aneurysm Treatment Device”) discloses an aneurysm treatment system including a conduit with a distal portion, a coupler slidably coupled to the distal portion, an occlusive member coupled to the coupler, and a securing member coupled to the conduit proximal to the coupler. U.S. patent applications 20210129275 (Nguyen et al., May 6, 2021, “Devices, Systems, and Methods for Treating Aneurysms”) and 20230311254 (Nguyen et al., Oct. 5, 2023, “Devices, Systems, and Methods for Treating Aneurysms”) disclose methods of manufacturing an aneurysm occlusion device including shaping a mesh with a forming assembly comprising multiple forming members, a mandrel, and/or one or more coupling elements.

U.S. patent application 20210128162 (Rhee et al., May 6, 2021, “Devices, Systems, and Methods for Treatment of Intracranial Aneurysms”) discloses delivering an occlusive member to an aneurysm cavity and transforming a shape of the occlusive member within the cavity, including delivering an embolic element between the occlusive member and the aneurysm wall. U.S. patent applications 20210128169 (Li et al., May 6, 2021, “Devices, Systems, and Methods for Treatment of Intracranial Aneurysms”), 20210153872 (Nguyen et al., May 27, 2021, “Devices, Systems, and Methods for Treatment of Intracranial Aneurysms”), 20230294223 (Li et al., Sep. 21, 2023, “Devices, Systems, and Methods for Treatment of Intracranial Aneurysms”), and 20230373040 (Nguyen et al., Nov. 23, 2023, “Devices, Systems, and Methods for Treatment of Intracranial Aneurysms”) disclose delivering an occlusive member to an aneurysm cavity and deforming a shape of the occlusive member via introduction of an embolic element to a space between the occlusive member and the aneurysm wall.

U.S. patent application 20210128160 (Li et al., May 6, 2021, “Systems and Methods for Treating Aneurysms”) discloses delivering an occlusive member to an aneurysm sac in conjunction with an embolic element. U.S. patent application 20210128165 (Pulugurtha et al., May 6, 2021, “Systems and Methods for Treating Aneurysms”) discloses an occlusive member configured to be positioned within an aneurysm sac and a distal conduit coupled to the occlusive member and having a first lumen extending therethrough. U.S. patent application 20210128167 (Patel et al., May 6, 2021, “Systems and Methods for Treating Aneurysms”) discloses delivering an occlusive member to an aneurysm sac in conjunction with an embolic element. U.S. patent applications 20210128168 (Nguyen et al., May 6, 2021. “Systems and Methods for Treating Aneurysms”) and 20230023511 (Nguyen et al., Jan. 26, 2023. “Systems and Methods for Treating Aneurysms”) disclose delivering an occlusive member to an aneurysm sac in conjunction with an embolic element.

U.S. patent application 20220031334 (Aguilar, Feb. 3, 2022, “Expandable Devices for Treating Body Lumens”) discloses an occlusive device comprising an expandable mesh including an outer mesh and an inner mesh disposed within the outer mesh, a connection portion positioned at or within an inner cavity of the inner mesh and configured to be detachably coupled to a delivery member. U.S. Pat. No. 11,685,007 (Li et al., Jun. 27, 2023, “Devices, Systems, and Methods for Treatment of Intracranial Aneurysms”) discloses delivering an occlusive member to an aneurysm cavity and deforming a shape of the occlusive member via introduction of an embolic element to a space between the occlusive member and the aneurysm wall. U.S. patent application 20230225735 (Kulak et al., Jul. 20, 2023, “Expandable Devices for Treating Body Lumens”) discloses an occlusive device comprising a mesh having a low-profile state for intravascular delivery to the aneurysm and a deployed state, wherein the mesh comprises a tubular mesh configured to curve along its longitudinal dimension when implanted in an aneurysm cavity.

U.S. patent application 20230277184 (Rashidi, Sep. 7, 2023, “Occlusive Devices With Thrombogenic Inserts”) discloses an occlusive device for treating an aneurysm includes an expandable mesh configured to span a neck of the aneurysm, wherein expandable mesh can include an upper wall, a lower wall, and an insert between the upper and lower walls. U.S. patent application 20230301660 (Sonmez et al., Sep. 28, 2023, “Systems and Methods for Treating Aneurysms”) discloses delivering an occlusive member to an aneurysm sac in conjunction with an embolic element. U.S. patent application 20230404592 (Nageswaran, Dec. 21, 2023, “Implant with Intrasaccular and Intravascular Portions and Related Technology”) discloses technology configured for treating an aneurysm at a treatment location within a patient's vasculature at which first, second, and third blood vessels converge.

U.S. patent application 20240032941 (Shimizu et al., Feb. 1, 2024, “Embolic Material Delivery Device and Related Technology”) discloses an elongate conduit body defining an axial lumen through which the conduit body is configured to convey liquid embolic material toward the aneurysm. U.S. patent application 20240050099 (Pecor et al., Feb. 15, 2024, “Occlusive Devices for Treating Vascular Defects and Associated Systems and Methods”) discloses a plurality of braided filaments configured to be implanted in an aneurysm cavity. U.S. patent application 20240075565 (Li et al., Mar. 7, 2024, “Systems and Methods for Treating Aneurysms”) discloses delivering an occlusive member to an aneurysm sac in conjunction with an embolic element.

U.S. Pat. No. 8,998,947 (Aboytes et al., Apr. 7, 2015, “Devices and Methods for the Treatment of Vascular Defects”) discloses an expandable implant that with a first configuration in which the first portion and the second portion are substantially linearly aligned and a second configuration in which the second portion at least partially overlaps the first portion. U.S. patent application 20200205841 (Aboytes et al., Jul. 2, 2020, “Devices, Systems, and Methods for the Treatment of Vascular Defects”) and U.S. patent application 20210378681 (Aboytes et al., Dec. 9, 2021, “Devices, Systems, and Methods for the Treatment of Vascular Defects”) disclose aneurysm occlusion devices with a first configuration in which a first portion and a second portion are substantially linearly aligned and a second configuration in which the second portion at least partially overlaps the first portion. U.S. patent application 20210244420 (Aboytes et al., Aug. 12, 2021, “Devices and Methods for the Treatment of Vascular Defects”) discloses aneurysm occlusion devices with a first configuration in which a first portion and a second portion are substantially linearly aligned and a second configuration in which the second portion at least partially overlaps the first portion.

U.S. patent application 20220031334 (Aguilar, Feb. 3, 2022. “Expandable Devices for Treating Body Lumens”) discloses an occlusive device comprising an expandable mesh including an outer mesh and an inner mesh disposed within the outer mesh. U.S. patent application 20230240686 (Ashby et al., Aug. 3, 2023, “Occlusive Devices with Spiral Struts for Treating Vascular Defects”) discloses an occlusive device with a plurality of spiral struts. U.S. Pat. No. 11,185,335 (Badruddin et al., Nov. 30, 2021, “System for and Method of Treating Aneurysms”) discloses an apparatus for treating an aneurysm with an occlusion element disposed on a wire, wherein the occlusion element includes a cover for covering a neck of an aneurysm and an inner anchoring member. U.S. patent application 20170079661 (Bardsley et al., Mar. 23, 2017, “Occlusive Devices”), U.S. Pat. No. 10,314,593 (Bardsley et al., Jun. 11, 2019, “Occlusive Devices”), and U.S. patent application 20190269411 (Bardsley et al., Sep. 5, 2019, “Occlusive Devices”) disclose an implant with a single- or dual-layer braided body with variable porosity.

U.S. patent application 20110208227 (Becking, Aug. 25, 2011, “Filamentary Devices for Treatment of Vascular Defects”) discloses braid-balls for aneurysm occlusion. U.S. patent application 20120316598 (Becking et al., Dec. 13, 2012, “Multiple Layer Filamentary Devices for Treatment of Vascular Defects”) and U.S. Pat. No. 9,585,669 (Becking et al., Mar. 17, 2017, “Multiple Layer Filamentary Devices for Treatment of Vascular Defects”) disclose braid balls for aneurysm occlusion. U.S. Pat. No. 9,039,726 (Becking, May 26, 2015, “Filamentary Devices for Treatment of Vascular Defects”) discloses braid-balls for aneurysm occlusion. U.S. patent application 20200367904 (Becking et al., Nov. 26, 2020, “Multiple Layer Filamentary Devices for Treatment of Vascular Defects”) and U.S. patent application 20220022886 (Becking et al., Jan. 27, 2022, “Multiple Layer Filamentary Devices for Treatment of Vascular Defects”) disclose braid-balls suitable for aneurysm occlusion.

U.S. patent application 20210228214 (Bowman et al., Jul. 29, 2021, “Devices for Vascular Occlusion”) discloses a method of using and delivering an occlusive device. U.S. patent application 20230157696 (Carrillo, May 25, 2023, “Aneurysm Treatment Device and Associated Systems and Methods of Use”) discloses an aneurysm treatment device with a tip portion, a body portion, and a base portion. U.S. patent application 20230165587 (Carrillo, Jun. 1, 2023, “Expandable Devices for Treating Body Lumens”) discloses an expandable cage with a plurality of mesh stents which receives embolic material therein. U.S. patent application 20220370078 (Chen et al., Nov. 24, 2022, “Vaso-Occlusive Devices”) discloses a vaso-occlusive structure made with a gold-platinum-tungsten alloy. U.S. patent application 20230190292 (Choubey et al., Jun. 22, 2023, “Occlusive Devices with Petal-Shaped Regions for Treating Vascular Defects”) discloses an occlusive device for treating an aneurysm with a mesh formed from a tubular braid, including a petal-shaped region formed from a flattened section of the tubular braid.

U.S. patent application 20120165919 (Cox et al., Jun. 28, 2012, “Methods and Devices for Treatment of Vascular Defects”) and U.S. patent application 20140052233 (Cox et al., Feb. 20, 2014, “Methods and Devices for Treatment of Vascular Defects”) disclose an expandable wire body support structure having a low profile radially constrained state, an expanded relaxed state with a substantially spherical or globular configuration having a smooth outer surface, and a porous permeable layer comprising a braided wire occlusive mesh. U.S. patent application 20120283768 (Cox et al., Nov. 8, 2012, “Method and Apparatus for the Treatment of Large and Giant Vascular Defects”) discloses the deployment of multiple permeable shell devices within a single vascular defect. U.S. Pat. No. 11,179,159 (Cox et al., Nov. 23, 2021, “Methods and Devices for Treatment of Vascular Defects”) discloses a device comprising a first hub, a second hub, a support structure having a longitudinal axis, the support structure disposed between the first hub and the second hub, the support structure including a plurality of struts, and a layer of material disposed over the plurality of struts, wherein the first hub is cylindrical and connected to an end of each of the struts of the plurality of struts. U.S. patent application 20230107778 (Cox et al., Apr. 6, 2023, “Methods and Devices for Treatment of Vascular Defects”) discloses an expandable body comprising a plurality of elongate filamentary elements each having a first end and a second end, wherein the elements extend from a first end of the device to a second end of the device and back to the first end of the device. U.S. patent application 20110022149 (Cox et al., Jan. 27, 2011, “Methods and Devices for Treatment of Vascular Defects”) discloses a device with first ends secured to a first ring and second ends secured to a second ring with the first and second rings being disposed substantially concentric to the longitudinal axis.

U.S. patent application 20200289125 (Dholakia et al., Sep. 17, 2020, “Filamentary Devices Having a Flexible Joint for Treatment of Vascular Defects”) discloses an implant with a first permeable shell having a proximal end with a concave or recessed section and a second permeable shell having a convex section that mates with the concave or recessed section. U.S. patent application 20210282785 (Dholakia et al., Sep. 16, 2021, “Devices Having Multiple Permeable Shells for Treatment of Vascular Defects”) a device with a plurality of permeable shells connected by a plurality of coils.

U.S. patent applications 20140135811 (Divino et al., May 15, 2014, “Occlusive Devices”), 20140135812 (Divino et al., May 15, 2014, “Occlusive Devices”), 20190282242 (Divino et al., Sep. 19, 2019, “Occlusive Devices”), and 20190290286 (Divino et al., Sep. 26, 2019, “Occlusive Devices”) and also U.S. Pat. No. 10,327,781 (Divino et al., Jun. 25, 2019, “Occlusive Devices”) disclose multiple expandable structures, wherein each of the expandable structures has a unique shape or porosity profile. U.S. patent application 20170281194 (Divino et al., Oct. 5, 2017, “Embolic Medical Devices”) discloses an occlusive device with an elongate member having opposing first and second side edges which extend longitudinally along the member and a member width, wherein this member has a collapsed configuration in which the first and second side edges are curled toward each other about a longitudinal axis of the member. U.S. patent application 20190343532 (Divino et al., Nov. 14, 2019, “Occlusive Devices”) discloses a device with at least one expandable structure which is adapted to transition from a compressed configuration to an expanded configuration when released into an aneurysm.

U.S. patent application 20200155333 (Franano et al., May 21, 2020, “Ballstent Device and Methods of Use”) discloses a rounded, thin-walled, expandable metal structure and a flexible, elongated delivery device. U.S. Pat. No. 11,013,516 (Franano et al., May 25, 2021, “Expandable Body Device and Method of Use”) discloses a single-lobed, thin-walled, expandable body comprising gold, platinum, or silver. U.S. Pat. No. 11,033,275 (Franano et al., Jun. 15, 2021, “Expandable Body Device and Method of Use”) discloses devices, designs, methods of manufacturing and using hollow gold structures that can be folded, wrapped, and compressed. U.S. patent application 20210275187 (Franano et al., Sep. 9, 2021. “Expandable Body Device and Method of Use”) discloses medical devices comprising a single-lobed, thin-walled, expandable body. U.S. patent application 20190053811 (Garza et al., Feb. 21, 2019, “Flow Attenuation Device”) and U.S. Pat. No. 11,071,551 (Garza et al., Jul. 27, 2021, “Flow Attenuation Device”) disclose an embolic device for treating aneurysms with a desired porosity only at discrete sections.

U.S. patent application 20190365385 (Gorochow et al., Dec. 5, 2019, “Aneurysm Device and Delivery System”) and U.S. Pat. No. 10,939,915 (Gorochow et al., Mar. 9, 2021, “Aneurysm Device and Delivery System”) discloses a braid, wherein translating the braid causes a delivery portion to expand and form a distal sack as well as invert into itself. U.S. patent application 20200113576 (Gorochow et al., Apr. 16, 2020, “Folded Aneurysm Treatment Device and Delivery Method”) and U.S. patent application 20210196284 (Gorochow et al., Jul. 1, 2021, “Folded Aneurysm Treatment Device and Delivery Method”) disclose an implant having a braided section that folds to form an outer occlusive sack extending across a neck of an aneurysm to engage a wall of the aneurysm from within a sac of the aneurysm and an inner occlusive sack forming a trough nested within the outer occlusive sack. The implant can be closed at one or more of the braid ends to define a substantially enclosed bowl-shaped volume.

U.S. Pat. No. 10,653,425 (Gorochow et al., May 19, 2020, “Layered Braided Aneurysm Treatment Device”), U.S. patent application 20200367893 (Xu et al., Nov. 26, 2020, “Layered Braided Aneurysm Treatment Device”), U.S. patent application 20200367898 (Gorochow et al., Nov. 26, 2020, “Layered Braided Aneurysm Treatment Device”), U.S. Pat. No. 11,413,046 (Xu et al., Aug. 16, 2022, “Layered Braided Aneurysm Treatment Device”), and U.S. patent application 20200367900 (Pedroso et al., Nov. 26, 2020. “Layered Braided Aneurysm Treatment Device With Corrugations”) disclose a tubular braid comprising an open end, a pinched end, and a predetermined shape; wherein, in the predetermined shape, the tubular braid comprises: a first segment extending from the open end to a first inversion, a second segment encircled by the open end such that the second segment is only partially surrounded by the first segment and extending from the first inversion to a second inversion, and a third segment surrounded by the second segment and extending from the second inversion to the pinched end.

U.S. patent application 20210085333 (Gorochow et al., Mar. 25, 2021, “Inverting Braided Aneurysm Treatment System and Method”), U.S. Pat. No. 11,278,292 (Gorochow et al., Mar. 22, 2022, “Inverting Braided Aneurysm Treatment System and Method”), and U.S. patent application 20220104829 (Gorochow et al., Apr. 7, 2022, “Inverting Braided Aneurysm Treatment System and Method”) disclose a tubular braid with an intrasaccular section, an intravascular section, a pinched section, and a predetermined shape. U.S. patent application 20210169495 (Gorochow et al., Jun. 10, 2021, “Intrasaccular Inverting Braid with Highly Flexible Fill Material”) and U.S. Pat. No. 11,602,350 (Gorochow et al., Mar. 14, 2023, “Intrasaccular Inverting Braid with Highly Flexible Fill Material”) disclose a tubular braided implant which is delivered as a single layer braid, inverted into itself during deployment to form at least two nested sacks and includes additional braid material that can fill the innermost sack.

U.S. patent application 20210186518 (Gorochow et al., Jun. 24, 2021, “Implant Having an Intrasaccular Section and Intravascular Section”) and U.S. Pat. No. 11,457,926 (Gorochow et al., Oct. 4, 2022, “Implant Having an Intrasaccular Section and Intravascular Section”) disclose a tubular braid with an intrasaccular section, an intravascular section, a pinched section, and a predetermined shape. U.S. Pat. No. 11,058,430 (Gorochow et al., Jul. 13, 2021, “Aneurysm Device and Delivery System”) discloses a braid with a proximal expandable portion for positioning inside an aneurysm and sealing across the neck of the aneurysm. U.S. Pat. No. 11,076,861 (Gorochow et al., Aug. 3, 2021, “Folded Aneurysm Treatment Device and Delivery Method”) discloses an implant with a fold which defines an annular ridge and a radiopaque marker band. U.S. patent application 20210106338 (Gorochow, Apr. 15, 2021, “Spiral Delivery System for Embolic Braid”) discloses a braided implant having a spiral segment. U.S. patent application 20210145449 (Gorochow, May 20, 2021, “Implant Delivery System with Braid Cup Formation”) discloses an implant system with an engagement wire, a pull wire, and a braided implant having a distal ring thereon. U.S. patent application 20210169498 (Gorochow, Jun. 10, 2021, “Delivery of Embolic Braid”) discloses a braided implant delivery system which attaches a braided implant having a band to a delivery tube, positions the braided implant within an aneurysm, and then releases the band from the delivery tube. U.S. Pat. No. 11,051,825 (Gorochow, Jul. 6, 2021, “Delivery System for Embolic Braid”) discloses a braided implant which is attached to a releasing component that can be detachably engaged with a delivery tube and a pull wire.

U.S. patent application 20210338250 (Gorochow et al., Nov. 4, 2021, “Intrasaccular Flow Diverter”) and U.S. Pat. No. 11,523,831 (Gorochow et al., Dec. 13, 2022, “Intrasaccular Flow Diverter”) disclose an interior fill braid physically which is inverted over itself to form a proximal inverted end and an opposite free end and a dome braid disposed distally of and secured to the interior fill braid. U.S. patent application 20220202425 (Gorochow et al., Jun. 30, 2022, “Semispherical Braided Aneurysm Treatment System and Method”) discloses a tubular braid with three segments and two inversions, one of the three segments extending between the two inversions and forming a sack. U.S. patent application 20210338247 (Gorochow, Nov. 4, 2021, “Double Layer Braid”) discloses a double layered braid for treating an aneurysm. U.S. Pat. No. 11,583,282 (Gorochow et al., Feb. 21, 2023, “Layered Braided Aneurysm Treatment Device”) discloses a method for shaping a tubular braid into a predetermined shape. U.S. patent application 20180070955 (Greene et al., Mar. 15, 2018, “Embolic Containment”) discloses a method of treating a neurovascular arteriovenous malformation with liquid embolic and dimethyl sulfoxide.

U.S. patent applications 20150313605 (Griffin, Nov. 5, 2015, “Occlusion Device”) and 20190059909 (Griffin, Feb. 28, 2019, “Occlusion Device”) and also U.S. Pat. No. 10,130,372 (Griffin, Nov. 20, 2018, “Occlusion Device”) and U.S. Pat. No. 11,389,174 (Griffin, Jul. 19, 2022, “Occlusion Device”) disclose an occlusion device with a substantially solid marker having a proximal end, and a distal end; and a low profile resilient mesh body attached to the distal end of the marker. U.S. patent application 20170156734 (Griffin, Jun. 8, 2017, “Occlusion Device”), U.S. Pat. No. 10,285,711 (Griffin, May 14, 2019, “Occlusion Device”), U.S. patent application 20190269414 (Griffin, Sep. 5, 2019, “Occlusion Device”), U.S. patent application 20210153871 (Griffin, May 27, 2021, “Occlusion Device”), and U.S. patent application 20220313274 (Griffin, Oct. 6, 2022, “Occlusion Device”) disclose a continuous compressible mesh structure comprising axial mesh carriages configured end to end, wherein each end of each carriage is a pinch point in the continuous mesh structure. U.S. Pat. No. 11,471,162 (Griffin, Oct. 18, 2022, “Occlusion Device”) discloses an occlusion device for implantation into a body lumen or aneurysm which has a continuous compressible mesh structure comprising axial mesh carriages configured end to end, wherein each end of each carriage is a pinch point in the continuous mesh structure.

U.S. patent application 20200187953 (Hamel et al., Jun. 18, 2020, “Devices, Systems, and Methods for the Treatment of Vascular Defects”) discloses a mesh comprising a first end portion, a second end portion, and a length extending between the first and second end portions, and a first lateral edge, a second lateral edge, and a width extending between the first and second lateral edges. U.S. patent application 20210353299 (Hamel et al., Nov. 18, 2021, “Devices, Systems, and Methods for the Treatment of Vascular Defects”) discloses a mesh that is curved along its length with an undulating contour across at least a portion of one or both of its length or its width. U.S. patent application 20220330947 (Henkes et al., Oct. 20, 2022, “Implant for the Treatment of Aneurysms”) discloses an implant which is rolled up relative to a radial axis in order to form a balled-up configuration.

U.S. patent application 20140358178 (Hewitt et al., Dec. 4, 2014, “Filamentary Devices for Treatment of Vascular Defects”), U.S. Pat. No. 9,078,658 (Hewitt et al., Jul. 14, 2015, “Filamentary Devices for Treatment of Vascular Defects”), U.S. patent application 20160249934 (Hewitt et al., Sep. 1, 2016, “Filamentary Devices for Treatment of Vascular Defects”), U.S. Pat. No. 9,955,976 (Hewitt et al., May 1, 2018, “Filamentary Devices for Treatment of Vascular Defects”), U.S. patent application 20180206849 (Hewitt et al., Jul. 26, 2018, “Filamentary Devices for the Treatment of Vascular Defects”), U.S. patent application 20210007754 (Milhous et al., Jan. 14, 2021, “Filamentary Devices for Treatment of Vascular Defects”), U.S. Pat. No. 10,939,914 (Hewitt et al., Mar. 9, 2021, “Filamentary Devices for the Treatment of Vascular Defects”), and U.S. patent application 20210275184 (Hewitt et al., Sep. 9, 2021, “Filamentary Devices for Treatment of Vascular Defects”) disclose occlusion devices with permeable shells made of woven braided mesh having a variable mesh density and/or porosity. U.S. patent application 20160249935 (Hewitt et al., Sep. 1, 2016, “Devices for Therapeutic Vascular Procedures”), U.S. patent application 20160367260 (Hewitt et al., Dec. 22, 2016, “Devices for Therapeutic Vascular Procedures”), U.S. Pat. No. 9,629,635 (Hewitt et al., Apr. 25, 2017, “Devices for Therapeutic Vascular Procedures”), and U.S. patent application 20170128077 (Hewitt et al., May 11, 2017, “Devices for Therapeutic Vascular Procedures”) disclose a self-expanding resilient permeable shell and a metallic coil secured to the distal end of the permeable shell.

U.S. Pat. No. 9,492,174 (Hewitt et al., Nov. 15, 2016, “Filamentary Devices for Treatment of Vascular Defects”), U.S. patent application 20170095254 (Hewitt et al., Apr. 6, 2017, “Filamentary Devices for Treatment of Vascular Defects”), U.S. Pat. No. 10,136,896 (Hewitt et al., Nov. 27, 2018, “Filamentary Devices for Treatment of Vascular Defects”), U.S. patent application 20190192166 (Hewitt et al., Jun. 27, 2019, “Filamentary Devices for Treatment of Vascular Defects”), U.S. patent application 20200289124 (Rangwala et al., Sep. 17, 2020, “Filamentary Devices for Treatment of Vascular Defects”), U.S. Pat. No. 10,813,645 (Hewitt et al., Oct. 27, 2020, “Filamentary Devices for Treatment of Vascular Defects”), and U.S. patent application 20210106337 (Hewitt et al., Apr. 15, 2021, “Filamentary Devices for Treatment of Vascular Defects”) disclose a self-expanding permeable shell having a radially constrained elongated state configured for delivery within a catheter lumen, an expanded state with a globular and longitudinally shortened configuration relative to the radially constrained state, and a plurality of elongate filaments that are woven together.

U.S. patent application 20190223881 (Hewitt et al., Jul. 25, 2019, “Devices for Therapeutic Vascular Procedures”) discloses a self-expanding resilient permeable shell made from elongate resilient filaments with a distal region that extends beyond the distal end of the permeable shell. U.S. patent application 20200289126 (Hewitt et al., Sep. 17, 2020, “Filamentary Devices for Treatment of Vascular Defects”), U.S. Pat. No. 11,317,921 (Hewitt et al., May 3, 2022, “Filamentary Devices for Treatment of Vascular Defects”), and U.S. patent application 20220257258 (Hewitt et al., Aug. 18, 2022, “Filamentary Devices for Treatment of Vascular Defects”) disclose a permeable shell or mesh with a stiffer proximal portion at the neck of an aneurysm. U.S. patent application 20220192678 (Hewitt et al., Jun. 23, 2022, “Filamentary Devices for Treatment of Vascular Defects”) discloses an implant having a first permeable shell having a proximal hub and an open distal end and a second permeable shell having a distal hub and an open proximal end. U.S. patent application 20220257260 (Hewitt et al., Aug. 18, 2022, “Filamentary Devices for Treatment of Vascular Defects”) discloses an implant having multiple mesh layers. U.S. patent application 20230114169 (Hewitt et al., Apr. 13, 2023, “Devices for Treatment of Vascular Defects”) discloses a permeable woven implant with a radially-constrained state for delivery within a catheter and an expanded state thereafter.

U.S. patent application 20230039246 (Hossan et al., Feb. 9, 2023, “Non-Braided Biodegrable Flow Diverting Device for Endovascular Treatment of Aneurysm and Associated Fabrication Method”) discloses a biodegradable flow-diverting device that regulates blood flow into an aneurysmal sac. U.S. patent application 20230263528 (Jones, Aug. 24, 2023, “Intrasacular Flow Diverter and Related Methods”) discloses an intrasaccular flow diverter with a plurality of wires which are coiled to form a collapsible, substantially spherical frame. U.S. patent application 20230252631 (Kashyap et al., Aug. 10, 2023, “Neural Network Apparatus for Identification, Segmentation, and Treatment Outcome Prediction for Aneurysms”) discloses using medical imaging and a neural network to predict outcomes from the potential use of one or more different intrasaccular implant devices. U.S. patent application 20210353300 (Kottenmeier et al., Nov. 18, 2021. “Systems and Methods for Treatment of Defects in the Vasculature”) discloses aneurysm occlusion methods and systems including an expandable stent.

U.S. patent application 20230225735 (Kulak et al., Jul. 20, 2023, “Expandable Devices for Treating Body Lumens”) discloses a tubular mesh which curves along its longitudinal dimension when implanted in an aneurysm cavity. U.S. patent application 20210137526 (Lee et al., May 13, 2021, “Embolic Devices for Occluding Body Lumens”) discloses an embolic device with a first segment forming a first three-dimensional structure, wherein the first three-dimensional structure defines a cavity; and a second segment forming a second three-dimensional structure; wherein the cavity of the first three-dimensional structure is configured to accommodate at least a majority of the second three-dimensional structure. U.S. patent application 20230017150 (Lee et al., Jan. 19, 2023, “Hydrogel Stent and Embolization Device for Cerebral Aneurysm”) discloses a hydrogel stent for occluding a cerebral aneurysm.

U.S. patent application 20210128169 (Li et al., May 6, 2021, “Devices, Systems, and Methods for Treatment of Intracranial Aneurysms”) and U.S. patent application 20210153872 (Nguyen et al., May 27, 2021, “Devices, Systems, and Methods for Treatment of Intracranial Aneurysms”) disclose delivering an occlusive member to an aneurysm cavity and deforming a shape of the occlusive member via introduction of an embolic element to a space between the occlusive member and an inner surface of the aneurysm wall. U.S. patent application 20210128160 (Li et al., May 6, 2021, “Systems and Methods for Treating Aneurysms”), U.S. patent application 20210128167 (Patel et al., May 6, 2021. “Systems and Methods for Treating Aneurysms”), U.S. patent application 20210128168 (Nguyen et al., May 6, 2021, “Systems and Methods for Treating Aneurysms”) and disclose delivering an occlusive member (e.g., an expandable braid) to an aneurysm sac in conjunction with an embolic element (e.g., coils, embolic material).

U.S. patent application 20190192168 (Lorenzo et al., Jun. 27, 2019, “Aneurysm Device and Delivery Method”) and U.S. Pat. No. 10,716,574 (Lorenzo et al., Jul. 21, 2020, “Aneurysm Device and Delivery Method”) discloses a self-expanding braid for treating an aneurysm, including a method for inverting and buckling a proximal segment. U.S. patent application 20190223878 (Lorenzo et al., Jul. 25, 2019, “Aneurysm Device and Delivery System”) and U.S. patent application 20200397447 (Lorenzo et al., Dec. 24, 2020, “Aneurysm Device and Delivery System”) discloses an expandable segment which radially expands inside an outer occlusive sack. U.S. patent application 20210007755 (Lorenzo et al., Jan. 14, 2021, “Intrasaccular Aneurysm Treatment Device with Varying Coatings”) discloses an aneurysm intrasaccular implant with coated regions. U.S. Pat. No. 10,905,430 (Lorenzo et al., Feb. 2, 2021, “Aneurysm Device and Delivery System”) discloses an expandable segment which radially expands inside an outer occlusive sack.

U.S. patent applications 20150272589 (Lorenzo, Oct. 1, 2015, “Aneurysm Occlusion Device”) and 20190008522 (Lorenzo, Jan. 10, 2019, “Aneurysm Occlusion Device”) and also U.S. Pat. No. 11,076,860 (Lorenzo, Aug. 3, 2021, “Aneurysm Occlusion Device”) disclose a tubular structure which is constrained by a control ring. U.S. patent application 20180242979 (Lorenzo, Aug. 30, 2018, “Aneurysm Device and Delivery System”) and U.S. Pat. No. 10,751,066 (Lorenzo, Aug. 25, 2020, “Aneurysm Device and Delivery System”) disclose a self-expanding braided tubular member. U.S. patent application 20200375606 (Lorenzo, Dec. 3, 2020, “Aneurysm Method and System”) discloses a braided implant which is invertible about the distal implant end. U.S. patent application 20210177429 (Lorenzo, Jun. 17, 2021, “Aneurysm Method and System”) discloses a vaso-occlusive device with at least two nested sacks. U.S. Pat. No. 11,123,077 (Lorenzo et al., Sep. 21, 2021, “Intrasaccular Device Positioning and Deployment System”) discloses implant deployment systems including a braided implant that can be detachably attached to a delivery tube by an expansion ring. U.S. patent application 20210330331 (Lorenzo, Oct. 28, 2021, “Aneurysm Occlusion Device”) and U.S. Pat. No. 11,154,302 (Lorenzo et al., Oct. 26, 2021, “Aneurysm Occlusion Device”) disclose an occlusion device with a substantially annular body disposed on the proximal end region of the device. U.S. patent application 20200038034 (Maguire et al., Feb. 6, 2020, “Vessel Occluder”) discloses a vessel occluder with an expandable mesh portion having a flexible membrane that expands within a cavity of the expandable mesh portion.

U.S. patent application 20130245667 (Marchand et al., Sep. 19, 2013, “Filamentary Devices and Treatment of Vascular Defects”) discloses a self-expanding resilient permeable shell wherein filaments are bundled and secured to each other at a proximal end. U.S. patent application 20160249937 (Marchand et al., Sep. 1, 2016, “Multiple Layer Filamentary Devices for Treatment of Vascular Defects”), U.S. Pat. No. 9,918,720 (Marchand et al., Mar. 20, 2018, “Multiple Layer Filamentary Devices for Treatment of Vascular Defects”), and U.S. Pat. No. 10,238,393 (Marchand et al., Mar. 26, 2019, “Multiple Layer Filamentary Devices for Treatment of Vascular Defects”) disclose a permeable shell and an inner structure configured to occlude blood flow. U.S. patent application 20180000489 (Marchand et al., Jan. 4, 2018, “Filamentary Devices for Treatment of Vascular Defects”) discloses a self-expanding resilient permeable shell having a plurality of elongate resilient filaments with a woven structure. U.S. Pat. No. 10,610,231 (Marchand et al., Apr. 7, 2020, “Filamentary Devices for Treatment of Vascular Defects”) discloses a self-expanding resilient permeable shell with a plurality of elongate resilient filaments with a woven structure, wherein the plurality of filaments includes small filaments and large filaments, and wherein the small filaments have a transverse dimension which is smaller than the transverse dimension of the large filaments. U.S. patent application 20200281603 (Marchand et al., Sep. 10, 2020, “Filamentary Devices for Treatment of Vascular Defects”) discloses a permeable shell including a really swell polymer.

U.S. Pat. No. 11,517,321 (Mauger et al., Dec. 6, 2022, “System and Methods for Embolized Occlusion of Neurovascular Aneurysms”) discloses an occlusion device with a reinforcing portion with no porosity. U.S. Pat. No. 11,589,872 (Mauger, Feb. 28, 2023, “Vascular Occlusion Devices Utilizing Thin Film Nitinol Foils”) discloses an implantable occlusion device wherein mesh components are wrapped around a support structure and slot that enables a disc to be wrapped around the support structure with overlapping portions. U.S. patent application 20230285031 (Mayer et al, Sep. 14, 2023, “Device for Restricting Blood Flow to Aneurysms”) discloses a non-occlusive device with a coilable section and a docking section. U.S. patent application 20220249098 (Milhous et al., Aug. 11, 2022, “Filamentary Devices for Treatment of Vascular Defects”) discloses a permeable implant with a plurality of scaffolding filaments. U.S. patent application 20230039773 (Monstadt et al., Feb. 9, 2023, “Implant for Treating Aneurysms”) discloses an implant which is preset to a specific structure.

U.S. Pat. No. 11,426,175 (Morita et al., Aug. 30, 2022, “Expansile Member”) discloses an occlusive system comprising: a catheter; a shell deliverable through the catheter, a delivery pusher detachably connected to the shell and configured to navigate the shell through the catheter, wherein the shell has a globular shaped portion. U.S. patent application 20210129275 (Nguyen et al., May 6, 2021, “Devices, Systems, and Methods for Treating Aneurysms”) discloses methods of manufacturing an occlusive device including conforming a mesh to a forming assembly and setting a shape of the mesh based on the forming assembly. U.S. patent application 20210275779 (Northrop, Sep. 9, 2021. “Actuating Elements for Bending Medical Devices”) discloses an actuating element causes a tube to bend. U.S. patent Ser. No. 11/498,165 (Patel et al., Nov. 15, 2022, “Systems and Methods for Treating Aneurysms”) discloses an occlusive implant with a conduit which receives a liquid embolic.

U.S. patent application 20210346032 (Patterson et al., Nov. 11, 2021, “Devices for Treatment of Vascular Defects”) discloses an expandable stent for placement in a parent vessel proximal, near, or adjacent an aneurysm. U.S. Pat. No. 11,607,226 (Pedroso et al., Mar. 21, 2023, “Layered Braided Aneurysm Treatment Device with Corrugations”) discloses an implant with a proximal inversion and two segments. U.S. Pat. No. 11,058,431 (Pereira et al., Jul. 13, 2021, “Systems and Methods for Treating Aneurysms”) discloses an inverted mesh tube having an outer layer and an inner layer, wherein the outer layer transitions to the inner layer at an inversion fold located at or adjacent to the distal end of the occlusion element. U.S. patent application 20170258473 (Plaza et al., Sep. 14, 2017, “Systems and Methods for Delivery of Stents and Stent-Like Devices”) discloses an elongate tubular member having a lumen, an expandable stent, and a delivery device which is placed in a cerebral vessel adjacent to an aneurysm.

U.S. patent applications 20060155323 (Porter et al., Jul. 13, 2006, “Intra-Aneurysm Devices”) and 20190298379 (Porter et al., Oct. 3, 2019, “Intra-Aneurysm Devices”) and also U.S. Pat. No. 10,265,075 (Porter et al., Apr. 23, 2019, “Intra-Aneurysm Devices”) disclose an occlusive device having a neck and a dome. U.S. patent application 20210052279 (Porter et al., Feb. 25, 2021, “Intra-Aneurysm Devices”) discloses a device including an upper member that sits against the dome of an aneurysm, a lower member that sits in the neck of the aneurysm, and a means of adjusting the overall dimensions of the device. U.S. patent application 20210128165 (Pulugurtha et al., May 6, 2021, “Systems and Methods for Treating Aneurysms”) and U.S. Pat. No. 11,305,387 (Pulugurtha et al., Apr. 19, 2022, “Systems and Methods for Treating Aneurysms”) disclose a distal conduit coupled to an occlusive member with a first lumen extending therethrough and a proximal conduit with a second lumen extending therethrough.

U.S. patent application 20220039804 (Rangwala et al., Feb. 10, 2022, “Flow-Diverting Implant and Delivery Method”) discloses a saddle-shaped braided mesh diverter that covers the neck of an aneurysm. U.S. Pat. No. 11,559,309 (Rangwala et al., Jan. 24, 2023, “Filamentary Devices for Treatment of Vascular Defects”) discloses a permeable implant whose proximal portion is stiffer. U.S. patent application 20230277184 (Rashidi et al., Sep. 7, 2023, “Occlusive Devices with Thrombogenic Inserts”) discloses an expandable mesh which spans a neck of the aneurysm with an insert configured to promote thrombosis.

U.S. patent application 20170079662 (Rhee et al., Mar. 23, 2017, “Occlusive Devices”) discloses an implant with a frame and a mesh component, wherein the mesh component has a first porosity and the frame has a second porosity. U.S. Pat. No. 10,478,194 (Rhee et al., Nov. 19, 2019, “Occlusive Devices”) and U.S. patent application 20200038032 (Rhee et al., Feb. 6, 2020, “Occlusive Devices”) disclose an implant with a frame and a mesh component, wherein the mesh component has a first porosity and the frame has a second porosity. U.S. patent application 20210128162 (Rhee et al., May 6, 2021, “Devices, Systems, and Methods for Treatment of Intracranial Aneurysms”) discloses introduction of an embolic element to a space between an occlusive member and an inner surface of the aneurysm wall.

U.S. patent application 20140330299 (Rosenbluth et al., Nov. 6, 2014, “Embolic Occlusion Device and Method”), U.S. patent application 20180303486 (Rosenbluth et al., Oct. 25, 2018, “Embolic Occlusion Device and Method”), and U.S. patent application 20210259699 (Rosenbluth et al., Aug. 26, 2021, “Embolic Occlusion Device and Method”) disclose an occlusion device with a tubular braided member having a first end and a second end and extending along a longitudinal axis, the tubular braided member having a repeating pattern of larger diameter portions and smaller diameter portions arrayed along the longitudinal axis. U.S. patent applications 20160022445 (Ruvalcaba et al., Jan. 28, 2016, “Occlusive Device”) and 20190343664 (Ruvalcaba et al., Nov. 14, 2019, “Occlusive Device”) U.S. Pat. No. 11,389,309 (Ruvalcaba et al., Jul. 19, 2022, “Occlusive Device”) disclose an aneurysm embolization device having a single, continuous piece of material that is shape set into a plurality of distinct structural components.

U.S. patent application 20020169473 (Sepetka et al., Nov. 14, 2002, “Devices and Methods for Treating Vascular Malformations”) discloses a device with a primary coil to provide structural integrity and secondary windings to fill interstitial spaces. U.S. patent application 20080281350 (Sepetka et al., Nov. 13, 2008, “Aneurysm Occlusion Devices”) discloses an implantable occlusion device with a concave or cup-shaped shape after implantation. U.S. Pat. No. 8,597,320 (Sepetka et al., Dec. 3, 2013, “Devices and Methods for Treating Vascular Malformations”) discloses a method of filling an aneurysm by advancing a device with a proximal collar and a distal collar through a vascular system and then positioning the device within an aneurysm. U.S. patent application 20140200607 (Sepetka et al., Jul. 17, 2014, “Occlusive Device”), U.S. patent application 20190274691 (Sepetka et al., Sep. 12, 2019, “Occlusive Device”), and U.S. Pat. No. 11,045,203 (Sepetka et al., Jun. 29, 2021. “Occlusive Device”) disclose multiple sequentially deployed occlusive devices that are connected together to create an extended length. U.S. patent application 20210282784 (Sepetka et al., Sep. 16, 2021, “Occlusive Device”) discloses a device comprising a plurality of braided wires and an embolic coil.

U.S. patent application 20170224350 (Shimizu et al., Aug. 10, 2017, “Devices for Vascular Occlusion”), U.S. Pat. No. 10,729,447 (Shimizu et al., Aug. 4, 2020, “Devices for Vascular Occlusion”), U.S. patent application 20200323534 (Shimizu et al., Oct. 15, 2020, “Devices for Vascular Occlusion”), U.S. Pat. No. 10,980,545 (Bowman et al., Apr. 20, 2021, “Devices for Vascular Occlusion”), U.S. patent application 20210228214 (Bowman et al., Jul. 29, 2021, “Devices for Vascular Occlusion”), and U.S. patent application 20210228214 (Bowman et al., Jul. 29, 2021, “Devices for Vascular Occlusion”) disclose an occlusive device, an occlusive device delivery system, method of using, method of delivering an occlusive device, and method of making an occlusive device to treat various intravascular conditions. U.S. patent application 20230031965 (Sivapatham, Feb. 2, 2023, “Intrasaccular Stent Device for Aneurysm Treatment”) discloses a system for treating an aneurysm in a blood vessel comprising a catheter, a guidewire, a delivery wire, an intrasaccular stent/retaining device removably attached to the delivery wire, and endovascular coiling.

U.S. patent application 20200305886 (Soto Del Valle et al, Oct. 1, 2020, “Aneurysm Treatment Device”) and U.S. patent application 20220225997 (Soto Del Valle et al., Jul. 21, 2022, “Aneurysm Treatment Device”) disclose a device with an expandable sack with a free open end and an elongated looping portion. U.S. patent application 20200305885 (Soto Del Valle et al, Oct. 1, 2020, “Aneurysm Treatment Device”) discloses an occlusion device that expands to form a cup shape within an aneurysm sac. U.S. patent application 20200375607 (Soto Del Valle et al., Dec. 3, 2020, “Aneurysm Device and Delivery System”) discloses a method of expanding mesh segments to form an outer occlusive sack and an inner occlusive sack. U.S. Pat. No. 11,337,706 (Soto Del Valle et al., May 24, 2022, “Aneurysm Treatment Device”) discloses an implant having an elongated portion and an expandable braided sack portion.

U.S. patent application 20210282789 (Vu et al., Sep. 16, 2021, “Multiple Layer Devices for Treatment of Vascular Defects”) discloses a first permeable shell and a second permeable shell, where the second permeable shell sits within an interior cavity of the first permeable shell. U.S. patent application 20220175389 (Wallace et al., Jun. 9, 2022, “Vaso-Occlusive Devices Including a Friction Element”) discloses a vaso-occlusive implant with a friction element between a soft braided member and a coil. U.S. patent application 20200187952 (Walsh et al., Jun. 18, 2020, “Intrasaccular Flow Diverter for Treating Cerebral Aneurysms”) and U.S. patent application 20220151632 (Walsh et al., May 19, 2022, “Intrasaccular Flow Diverter for Treating Cerebral Aneurysms”) disclose a stabilizing frame with two parts, the first part sized to anchor within the sac of the aneurysm and the exterior part sized to anchor against a region of the blood vessel wall adjacent the aneurysm neck.

U.S. patent application 20200029973 (Walzman, Jan. 30, 2020, “Mash Cap for Ameliorating Outpouchings”) discloses an embolic device comprising a control element, a catheter element, a delivery microcatheter hypotube, a detachment element, a mesh disc, a distal opening, and at least one attached extension arm. U.S. patent application 20200405347 (Walzman, Dec. 31, 2020, “Mesh Cap for Ameliorating Outpouchings”) discloses a self-expandable occluding device which covers the neck of an outpouching and serves as a permanent embolic plug. U.S. Pat. No. 10,398,441 (Warner et al., Sep. 3, 2019, “Vascular Occlusion”) discloses a vascular treatment system with a containment device, a pusher, and a stopper ring. U.S. Pat. No. 11,166,731 (Wolfe et al., Nov. 9, 2021, “Systems and Methods for Treating Aneurysms”) discloses an inverted mesh tube having an outer layer and an inner layer, the outer layer transitioning to the inner layer at an inversion fold.

U.S. patent application 20200367906 (Xu et al., Nov. 26, 2020, “Aneurysm Treatment With Pushable Ball Segment”) and U.S. patent application 20230016312 (Xu et al., Jan. 19, 2023, “Aneurysm Treatment with Pushable Implanted Braid”) disclose a braided implant with a retractable dual proximal layer. U.S. patent application 20220087681 (Xu et al., Mar. 24, 2022, “Inverting Braided Aneurysm Implant with Dome Feature”) discloses an implant with a dome feature configured to press into aneurysm walls near the aneurysm's dome and facilitate securement of the braid across the aneurysm's neck. U.S. Pat. No. 11,497,504 (Xu et al., Nov. 15, 2022, “Aneurysm Treatment with Pushable Implanted Braid”) discloses a braided implant with a retractable dual proximal layer. U.S. Pat. No. 11,596,412 (Xu et al., Mar. 7, 2023, “Aneurysm Device and Delivery System”) discloses a braid with a proximal portion which goes across an aneurysm neck and an expandable distal portion. U.S. patent application 20230061363 (Yee et al., Mar. 2, 2023, “Embolic Device with Improved Neck Coverage”) discloses an embolic device with a flexible structure which has a series of alternating narrow portions and link portions.

U.S. patent application 20200367896 (Zaidat et al., Nov. 26, 2020, “Systems and Methods for Treating Aneurysms”) discloses an apparatus for treating an aneurysm in a blood vessel with a first tubular mesh having a first end and a second end coupled together at a proximal end of the occlusion element. U.S. Pat. No. 11,202,636 (Zaidat et al., Dec. 21, 2021, “Systems and Methods for Treating Aneurysms”), U.S. patent application 20220022884 (Wolfe et al., Jan. 27, 2022, “Systems and Methods for Treating Aneurysms”), and U.S. patent application 20220211383 (Pereira et al., Jul. 7, 2022, “Systems and Methods for Treating Aneurysms”) disclose an apparatus for treating an aneurysm including an occlusion element configured to be releasably coupled to an elongate delivery shaft and a distal end, a proximal end, and a longitudinal axis extending between the distal end and the proximal end. U.S. patent application 20220054141 (Zaidat et al., Feb. 24, 2022, “Systems and Methods for Treating Aneurysms”) discloses an apparatus for treating an aneurysm in a blood vessel with a first tubular mesh having a first end and a second end coupled together at a proximal end of the occlusion element.

### SUMMARY OF THE INVENTION

Disclosed herein is an intrasaccular aneurysm occlusion device including a mesh, net, lattice, or braid, wherein a proximal portion of the mesh, net, lattice, or braid has a single layer in a first configuration and multiple layers in a second configuration. The mesh, net, lattice, or braid has a globular shape which is collapsed, compressed, inverted, and/or folded into a bowl shape in an aneurysm sac in order to create a double-layer barrier across an aneurysm neck. There is opening in the mesh, net, lattice, or braid through which liquid embolic material is inserted into the aneurysm sac. In an example, insertion of the embolic material collapses, compresses, inverts, and/or folds the mesh, net, lattice, or braid from the first configuration to the second configuration.

### BRIEF INTRODUCTION TO THE FIGURES

FIG. 1 shows a device with a bowl-shaped neck bridge, a distal mesh, and embolic material.

FIGS. 2 through 5 show four sequential views of device with a spherical mesh which is collapsed into a bowl shape, a distal mesh, and embolic material.

FIG. 2 shows the device exiting a catheter into an aneurysm sac.

FIG. 3 shows the spherical mesh expanding.

FIG. 4 shows the spherical mesh collapsing into a bowl shape.

FIG. 5 shows the device after embolic material has been inserted.

FIG. 6 shows a half-torus neck bridge, a distal mesh, and embolic material.

FIG. 7 shows a toroidal neck bridge, a distal mesh, and embolic material.

FIG. 8 shows a bowl-shaped neck bridge, a distal mesh, and embolic material.

FIG. 9 shows four sequential views of a mesh which is inserted, radially-expanded, and then longitudinally-collapsed within an aneurysm sac.

FIG. 10 shows a double-layer bowl-shaped mesh with a valve through which embolic material is inserted into an aneurysm sac.

FIG. 11 shows a leaflet valve through which embolic material can be inserted into an aneurysm sac.

FIG. 12 shows an elastic annular valve through which embolic material can be inserted into an aneurysm sac.

FIG. 13 shows a rotational valve through which embolic material can be inserted into an aneurysm sac.

FIG. 14 shows a sliding valve through which embolic material can be inserted into an aneurysm sac.

FIG. 15 shows a pivoting valve through which embolic material can be inserted into an aneurysm sac.

FIG. 16 shows a plug mechanism which closes an opening through which embolic material has been inserted into an aneurysm sac.

FIG. 17 shows a bowl-shaped mesh wherein a first pinched end of a tubular mesh points outward in a proximal direction and a second pinched end of the tubular mesh points outward in a distal direction.

FIG. 18 shows a bowl-shaped mesh wherein a first pinched end of a tubular mesh points outward in a proximal direction and a second pinched end of the tubular mesh points inward in a proximal direction.

FIG. 19 shows a bowl-shaped mesh wherein a first pinched end of a tubular mesh points inward in a distal direction and a second pinched end of the tubular mesh points inward in a proximal direction.

FIG. 20 shows a bowl-shaped mesh wherein a first pinched end of a tubular mesh points inward in a distal direction and a second pinched end of the tubular mesh points outward in a distal direction.

FIG. 21 shows a bowl-shaped mesh wherein a first pinched end of a tubular mesh points inward in a distal direction and a second pinched end of the tubular mesh points outward in a distal direction, wherein the ends overlap.

FIG. 22 shows a bowl-shaped mesh wherein a first pinched end of a tubular mesh points outward in a proximal direction and a second pinched end of the tubular mesh points inward in a proximal direction, wherein the ends overlap.

FIG. 23 shows four sequential views of a parent vessel stent and an intrasaccular globular mesh which is compressed into a bowl-shaped mesh.

FIGS. 24 through 28 show five sequential views of the making and deployment of a bowl-shaped mesh and also embolic material.

FIG. 24 shows a tubular mesh.

FIG. 25 shows the ends of the tubular mesh being constrained.

FIG. 26 shows the tubular mesh being collapsed into a bowl-shaped mesh.

FIG. 27 shows embolic material being inserted into an aneurysm sac through an opening in the bowl-shaped mesh.

FIG. 28 shows the bowl-shaped mesh and the embolic material having been completely deployed.

FIGS. 29 through 32 show a proximal mesh being compressed from a globular to a bowl-shaped shape by movement of a wire and embolic material being inserted into a distal mesh.

FIG. 29 shows the mesh being inserted into an aneurysm sac.

FIG. 30 shows the mesh being radially-expanded into a globular shape.

FIG. 31 shows the mesh being collapsed into a bowl shape.

FIG. 32 shows embolic material having been inserted into the aneurysm sac.

FIGS. 33 through 36 show a proximal mesh being compressed from a globular to a bowl-shaped shape by movement of a wire and embolic material being inserted into an aneurysm sac.

FIG. 33 shows the mesh being inserted into an aneurysm sac.

FIG. 34 shows the mesh being radially-expanded into a globular shape.

FIG. 35 shows the mesh being collapsed into a bowl shape.

FIG. 36 shows embolic material having been inserted into the aneurysm sac.

FIGS. 37 through 40 show a proximal mesh being compressed from a globular to a bowl-shaped shape by the accumulation of embolic material in a distal mesh.

FIG. 37 shows the mesh being inserted into an aneurysm sac.

FIG. 38 shows the mesh being radially-expanded into a globular shape.

FIG. 39 shows the mesh being collapsed into a bowl shape.

FIG. 40 shows embolic material having been inserted into the aneurysm sac.

FIGS. 41 through 44 show a proximal mesh being compressed from a globular to a bowl-shaped shape by the accumulation of embolic material in an aneurysm sac.

FIG. 41 shows the mesh being inserted into an aneurysm sac.

FIG. 42 shows the mesh being radially-expanded into a globular shape.

FIG. 43 shows the mesh being collapsed into a bowl shape.

FIG. 44 shows embolic material having been inserted into the aneurysm sac.

FIGS. 45 through 48 show four sequential views of the formation and deployment of another example of a bowl-shaped mesh.

FIG. 45 shows a tubular mesh.

FIG. 46 shows the tubular mesh having been inverted into a bowl-shaped mesh.

FIG. 47 shows embolic material being inserted into an aneurysm sac through an opening in the bowl-shaped mesh.

FIG. 48 shows the bowl-shaped mesh and the embolic material having been completely deployed.

### DETAILED DESCRIPTION OF THE FIGURES

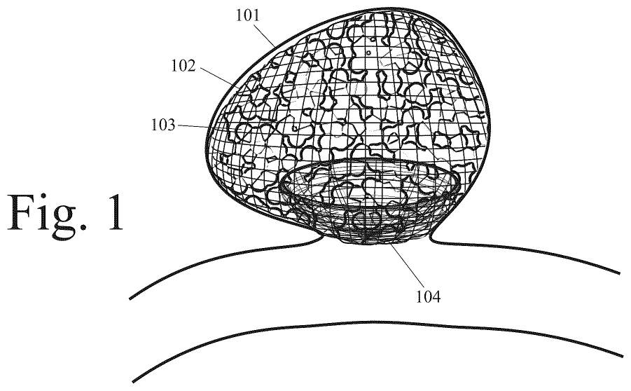

Before discussing the specific embodiments of this invention which are shown in FIGS. 1 through 48, this disclosure provides an introductory section which covers some of the general concepts, components, and methods which comprise this invention. Where relevant, these concepts, components, and methods can be applied as variations to the examples shown in FIGS. 1 through 48 which are discussed afterwards.

In an example, a resilient wider-than-neck part of an aneurysm occlusion device can comprise: a mesh, net, lattice, or braid whose proximal part comprises a single layer in a first configuration and two or more overlapping layers in a second configuration. In an example, the resilient wider-than-neck part can have a generally spherical shape which is collapsed into a generally hemispherical and/or bowl shape in an aneurysm sac in order to create a double-layer barrier across an aneurysm neck.

In an example, there can be an opening in the resilient wider-than-neck part of the aneurysm occlusion device. In an example, liquid embolic material can be inserted through the opening into the aneurysm sac. In an example, a closure mechanism can close the opening after the liquid embolic material has been inserted into the aneurysm sac. In an example, the closure mechanism can be selected from the group consisting of: tensile ring; drawstring; electromagnetic melting mechanism; insertable plug; clamp; hydrogel plug; and bioactive congealing plug. In an example, the device can further comprise multiple delivery lumens or catheters. In an example, a first delivery lumen or catheter can deliver the resilient wider-than-neck part of the device and a second delivery lumen or catheter can deliver the embolic material.

In an example, a resilient wider-than-neck portion of an aneurysm occlusion device can comprise: a mesh, net, or lattice which is inserted into an aneurysm sac; wherein the mesh, net, or lattice has a first configuration in which a proximal portion of the mesh, net, or lattice has a single layer; and wherein the mesh, net, or lattice has a second configuration in which the proximal portion of the mesh, net, or lattice has two or more layers. In an example, the resilient wider-than-neck portion can be collapsed, compressed, and/or folded from the first configuration to the second configuration. In an example, the resilient wider-than-neck portion can have a generally spherical or ellipsoidal shape in the first configuration. In an example, the resilient wider-than-neck portion can have a bowl or cup shape in the second configuration.

In an example, there can be an opening in the resilient wider-than-neck portion. In an example, liquid embolic material can be inserted through the opening into the aneurysm sac. In an example, a closure mechanism can close the opening after the liquid embolic material has been inserted into the aneurysm sac. In an example, the closure mechanism can be selected from the group consisting of: tensile ring; drawstring; electromagnetic melting mechanism; insertable plug; clamp; hydrogel plug; and bioactive congealing plug. In an example, the device can further comprise multiple delivery lumens or catheters. In an example, a first delivery lumen or catheter can deliver the resilient wider-than-neck portion of the device and a second delivery lumen or catheter can deliver the embolic material.

In an example, an aneurysm occlusion device can comprise: a mesh, net, or lattice which is inserted into an aneurysm sac; wherein the mesh, net, or lattice is made with braided or woven filaments, wires, tubes, or strands; wherein first ends of the filaments, wires, tubes, or strands converge at a proximal hub in the first configuration; wherein second ends of the filaments, wires, tubes, or strands converge at a distal hub in the first configuration; and wherein the distal hub is closer to the proximal hub in the second configuration than in the first configuration. In an example, the distal hub does not contact the proximal hub in the first configuration, but the distal hub does contact the proximal hub in the second configuration.

In an example, a resilient wider-than-neck portion of an aneurysm occlusion device can have a generally spherical shape which is collapsed into a generally hemispherical and/or bowl shape in an aneurysm sac in order to create a double-layer barrier across an aneurysm neck. In an example, this can be done when a device operator pulls on a wire, cable, or cord connected to the distal surface of the resilient wider-than-neck portion. In an example, a resilient wider-than-neck portion of a device can be a multi-layer polymer bowl or hemisphere.

In an example, a resilient wider-than-neck portion can comprise a multi-layer bowl or hemisphere. In an example, a portion can have a metal layer and a polymer layer. In an example, an inner layer can be metal and an outer layer can be made from a polymer. In an example, a resilient wider-than-neck portion of a device can have a biologically-active layer which encourages cell growth for more thorough embolization of the aneurysm neck. In an example, a resilient wider-than-neck portion can have multiple layers of material with different mesh or braid directions, different porosity levels, different elasticity levels, and/or different rigidity levels.

In an example, a resilient wider-than-neck portion of a device can have a single layer in its first configuration and multiple layers in its second configuration. In an example, a resilient wider-than-neck portion of a device can be folded and/or curved back on itself in its second configuration so as to have multiple layers in its second configuration. In an example, the proximal part of a resilient wider-than-neck portion of a device can have a single layer as it is transported through a delivery lumen to an aneurysm sac and can have two overlapping layers after it is released from the lumen. In an example, it can be folded after expansion within the aneurysm sac. In an example, a resilient wider-than-neck portion of a device can be a mesh, net, lattice, or braid whose proximal part comprises a single layer in its first configuration (as it is transported through a delivery lumen to an aneurysm sac) and two or more overlapping layers in its second configuration (after the resilient wider-than-neck portion is released from the lumen and deployed within the aneurysm sac).

In an example, a resilient wider-than-neck portion of a device can have an opening through which embolic members are inserted. In an example, this opening can have a one-way valve which enables embolic members to be inserted into the flexible sac-filling portion of a device but not escape out. In an example, an opening can be manually and/or remotely changed from a first (open) configuration to a second (closed) configuration. In an example, the device can further comprise a closure mechanism (which closes an opening) selected from the group consisting of: tensile ring; drawstring; electromagnetic melting mechanism; insertable plug; clamp; hydrogel plug; and bioactive congealing plug.

In an example, embolic members can be pieces of gel or foam. In an example, embolic members can be embolic coils. In an example, embolic members can be selected from the group consisting of: beads or microspheres; compressible balls; congealing gel; embolic coils; fibers;

hydrogel pieces; microspheres; microsponges; pieces of foam; pieces of gel; rigid balls; and wires. In an example, embolic members can be 3D polygonal pieces of gel or foam.

In an example, a device can comprise multiple delivery lumens or catheters. In an example, a first delivery lumen (or catheter) can deliver a resilient wider-than-neck portion of a device and a second delivery lumen (or catheter) can deliver embolic members (or material). In an example, a device can further comprise a delivery lumen (such as a catheter) with a rotatable distal end, wherein rotation of the distal end changes the orientation of a resilient wider-than-neck portion delivered into an aneurysm sac.

In an example, a neck bridge can be a multi-layer neck bridge which is made by folding and/or compressing a generally spherical or ellipsoidal stent, lattice, mesh, or framework within the aneurysm sac. In an example, a neck bridge can be a multi-layer neck bridge which is made by folding and/or compressing a generally spherical or ellipsoidal stent, lattice, mesh, or framework into a multi-layer bowl or cup shaped stent, lattice, mesh, or framework.

In an example, a neck bridge can self-expand within an aneurysm sac. In an example, a neck bridge can be a braided or woven stent, lattice, mesh, net, and/or framework. In an example, a neck bridge can be a 3D printed stent, lattice, mesh, and/or framework. In an example, a neck bridge can be 3D printed as one, continuous piece of mesh. In an example, a neck bridge can be a multi-layer neck bridge which is made by folding and/or compressing a generally spherical or ellipsoidal stent, lattice, mesh, or framework into a multi-layer bowl or cup shaped stent, lattice, mesh, or framework. In an example, a neck bridge can be a multi-layer neck bridge which is made by folding and/or compressing a generally spherical or ellipsoidal stent, lattice, mesh, or framework within the aneurysm sac. In an example, a neck bridge can be a multi-layer neck bridge which is made by folding, twisting, and/or inverting a generally tubular stent, lattice, mesh, or framework into a multi-layer bowl or cup shaped stent, lattice, mesh, or framework.

In an example, an expandable proximal member of an aneurysm occlusion device can have a toroidal shape, bowl shape, convex lens shape, cup shape, paraboloid (of revolution) shape, inverted umbrella shape, or hemispherical shape. In an example, an expandable proximal member can have an annular shape, cylindrical shape doughnut shape, ring shape, tire shape, or wheel shape. In an example, an expandable proximal member can be a spherical stent which has been collapsed into a hemispherical shape. In an example, an expandable proximal member can comprise a spherical stent which is collapsed into a hemisphere in order to create a double-layer barrier near an aneurysm neck.

In an example, an expandable proximal member of an aneurysm occlusion device can further comprise an opening through which embolic members are introduced, wherein the closure mechanism is configured to be actuated to reduce a size of the opening. In an example, a hole (e.g. opening) in an expandable proximal member can be closed after an aneurysm sac has been filled with embolic members inserted through the hole. In an example, a closure mechanism can be integrated into an expandable proximal member to prevent embolic members from escaping through the opening by which they were inserted. In an example, a closure mechanism can be remotely actuated by a device user to reduce a size of the opening. In an example, a closure mechanism can comprise a one-way valve that automatically lets embolic members into the aneurysm sac, but does not let them out. In an example, a closure mechanism can include a cord which is pulled from a remote location outside the body to detach an expandable proximal member from a catheter and close an opening in the expandable proximal member.

In an example, an aneurysm occlusion device can comprise: (a) a catheter; (b) one or more embolic coils which are inserted through the catheter into an aneurysm sac; (c) a braided Nitinol neck bridge (e.g. stent, framework, lattice, or mesh) which is inserted into the aneurysm sac in a first shape, then is expanded (e.g. self-expands) within the aneurysm sac into an ellipsoidal second shape, and then is compressed and/or folded into a bowl or cup third shape with a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an example, a proximal portion of a net or mesh can expand within an aneurysm sac into a spherical, prolate spheroidal, ellipsoidal, or ovaloidal shape and then be transformed (e.g. compressed) into a bowl, hemispherical, contact lens, paraboloid, or inverted umbrella shape. In an example, a proximal portion of a net or mesh can expand within an aneurysm sac into a spherical, prolate spheroidal, ellipsoidal, or ovaloidal shape and then be transformed (e.g. compressed) into a bowl, hemispherical, contact lens, paraboloid, or inverted umbrella shape by a user pulling (or pushing) a distal portion of the spherical, prolate spheroidal, ellipsoidal, or ovaloidal shape in a proximal direction.

In an example, an aneurysm occlusion device can comprise: (a) a catheter; (b) one or more embolic coils which are inserted through the catheter into an aneurysm sac; (c) a Nitinol neck bridge (e.g. stent, framework, lattice, or mesh) which is inserted into the aneurysm sac in a first shape, then expanded (e.g. self-expands) within the aneurysm sac into an ellipsoidal second shape, and then compressed and/or folded into a bowl or cup third shape with a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an example, an aneurysm occlusion device can comprise: (a) a catheter; (b) one or more embolic coils which are inserted through the catheter into an aneurysm sac; (c) a shaped neck bridge (e.g. stent, lattice, mesh, or framework) which is inserted into the aneurysm sac, is expanded (e.g. self-expands) into a substantially spherical shape within the aneurysm sac, and then is collapsed into a double-layer, upward-opening, bowl shape which substantially covers the inside of the aneurysm neck; (d) a (central) opening in the neck bridge through which the coils are inserted into the aneurysm sac; and (e) a closure mechanism which closes the opening after the coils have been inserted into the aneurysm sac.

In an example, a net or mesh which is inserted into an aneurysm sac can have a structure which is selected from the group consisting of: braided or woven shell, braided or woven structure, braided or woven tubular structure with inverted end portions, braided or woven tubular structure with tied ends, braided textile sphere, braided wire sphere, dual-layer braided or woven structure, hollow braided or woven structure, spherical braided or woven structure, and tubular braided or woven structure. In an example, a net or mesh which is inserted into an aneurysm sac can have a structure which is selected from the group consisting of: 3D-printed convex net or mesh, flexible metal mesh or net, metal hexagonal mesh or net, and polymer hexagonal mesh or net.

In an example, an aneurysm occlusion device can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck, wherein the expandable proximal member is a spherical stent which has been collapsed or folded into a double-layer hemispherical shape; (d) a (central) opening in the neck bridge through which the coils are inserted into the aneurysm sac; and (e) a closure mechanism which closes the opening after the coils have been inserted into the aneurysm sac.

In an example, a proximal portion of a net or mesh which is inserted into an aneurysm sac can have a structure which is selected from the group consisting of: 3D-printed convex proximal portion of a net or mesh, flexible metal mesh or net, metal hexagonal mesh or net, and polymer hexagonal mesh or net. In an example, a proximal portion of a net or mesh which is inserted into an aneurysm sac can have a structure which is selected from the group consisting of: containment bag, dual-layer body, flexible aneurysm liner, hollow framing structure, hollow shell structure, and thin-wall flexible metal sphere with holes. In an example, a proximal portion of a net or mesh which is inserted into an aneurysm sac can be a cellular lattice or a hollow array of biological cells.

In an example, an aneurysm occlusion device can comprise: (a) inserting a braided neck bridge (e.g. stent, lattice, mesh, or framework) into an aneurysm sac; (b) allowing the neck bridge to self-expand into a spherical or ellipsoidal first shape; (c) axially contracting, compressing, and/or folding the neck bridge from the first shape into a double-layer bowl or cup second shape; (d) inserting embolic coils into the aneurysm sac through an opening in the neck bridge; and (e) closing the opening in the neck bridge.

In an example, an aneurysm occlusion device can comprise: (a) a catheter; (b) one or more embolic coils which are inserted through the catheter into an aneurysm sac; (c) a shaped neck bridge (e.g. stent, lattice, mesh, or framework) which is inserted into the aneurysm sac, is expanded (e.g. self-expands) into a substantially ellipsoidal shape within the aneurysm sac, and then is collapsed into a double-layer, upward-opening, bowl shape which substantially covers the inside of the aneurysm neck; (d) a (central) opening in the neck bridge through which the coils are inserted into the aneurysm sac; and (e) a closure mechanism which closes the opening after the coils have been inserted into the aneurysm sac.

In an example, a proximal portion of a net or mesh can have a first level of elasticity, the distal portion of the net or mesh can have a second level of elasticity, and the second level can be greater than the first level. In an example, the proximal portion of a net or mesh can have a first level of flexibility, the distal portion of the net or mesh can have a second level of flexibility, and the second level can be greater than the first level. In an example, the proximal portion of a net or mesh can have a first level of conformability, the distal portion of the net or mesh can have a second level of conformability, and the second level can be greater than the first level.

In an example, a proximal portion of a net or mesh can have a first level of resilience, the distal portion of the net or mesh can have a second level of resilience, and the second level can be less than the first level. In an example, the proximal portion of a net or mesh can have a first level of radial strength, the distal portion of the net or mesh can have a second level of radial strength, and the second level can be less than the first level. In an example, the proximal portion of a net or mesh can have a first level of stiffness, the distal portion of the net or mesh can have a second level of stiffness, and the second level can be less than the first level.

In an example, an aneurysm occlusion device can comprise: (a) a catheter; (b) one or more embolic coils which are inserted through the catheter into an aneurysm sac; (c) a neck bridge (e.g. stent, framework, lattice, or mesh) which is inserted into the aneurysm sac in a first shape, then is expanded (e.g. self-expands) within the aneurysm sac into a spherical second shape, and then is compressed and/or folded into a hemispherical third shape with a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an example, an aneurysm occlusion device can have a first, compressed configuration as it travels through a catheter to an aneurysm. In an example, an aneurysm occlusion device can transition from the first, compressed configuration to a second, expanded, generally spherical configuration within the aneurysm sac. In an example, an aneurysm occlusion device can transition from the second, expanded, generally spherical configuration to a third, generally hemispherical configuration covering the interior of the aneurysm neck. In an example, an aneurysm occlusion device can form a double-thickness wire mesh which covers the aneurysm neck.

In an example, embolic members can be embolic coils or ribbons. In an example, embolic members can be pieces of foam or gel (such as hydrogel). In an example, embolic members can be microballs or microspheres. In an example, embolic members can be microsponges. In an example, embolic members can be filaments or yarns. In an example, liquid embolic material can be inserted into a net or mesh. In an example, liquid embolic material can be is inserted through an opening into an aneurysm sac and then congeal within the aneurysm sac. In an example, a closure mechanism can close the opening after the liquid embolic material has been inserted into the aneurysm sac.

In an example, an aneurysm occlusion device can comprise: (a) a catheter; (b) one or more embolic coils which are inserted through the catheter into an aneurysm sac; (c) a braided Nitinol neck bridge (e.g. stent, framework, lattice, or mesh) which is inserted into the aneurysm sac in a first shape, then is expanded (e.g. self-expands) within the aneurysm sac into a spherical second shape, and then is compressed and/or folded into a hemispherical third shape with a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an example, an aneurysm occlusion device can comprise: (a) a catheter; (b) one or more embolic coils which are inserted through the catheter into an aneurysm sac; (c) a neck bridge (e.g. stent, framework, lattice, or mesh) which is inserted into the aneurysm sac in a first shape, then is expanded (e.g. self-expands) within the aneurysm sac into an ellipsoidal second shape, and then is compressed and/or folded into a bowl or cup third shape with a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an example, an aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) within an aneurysm sac, expanding into a bowl or cup shape with a diameter which is larger than the diameter of the aneurysm neck, wherein the proximal half of the neck bridge has a first porosity level, wherein the distal half of the neck bridge has a second porosity level, and wherein the second porosity level is greater than the first porosity level; (d) a (central) opening in the neck bridge through which the coils are inserted into the aneurysm sac; and (e) a closure mechanism which closes the opening after the coils have been inserted into the aneurysm sac.

In an example, an aneurysm occlusion device can comprise: (a) a catheter; (b) one or more embolic coils which are inserted through the catheter into an aneurysm sac; (c) a neck bridge (e.g. stent, lattice, mesh, or framework) which is inserted into the aneurysm sac, is expanded (e.g. self-expands) into a substantially spherical shape within the aneurysm sac, and then is collapsed into a double-layer, upward-opening, hemispherical shape which substantially covers the inside of the aneurysm neck; (d) a (central) opening in the neck bridge through which the coils are inserted into the aneurysm sac; and (e) a closure mechanism which closes the opening after the coils have been inserted into the aneurysm sac.

In an example, an aneurysm occlusion device can comprise: (a) a catheter; (b) one or more embolic coils which are inserted through the catheter into an aneurysm sac; (c) a Nitinol neck bridge (e.g. stent, framework, lattice, or mesh) which is inserted into the aneurysm sac in a first shape, then expanded (e.g. self-expands) within the aneurysm sac into a spherical second shape, and then compressed and/or folded into a hemispherical third shape with a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an example, an aneurysm occlusion device can comprise: (a) a catheter; (b) one or more embolic coils which are inserted through the catheter into an aneurysm sac; (c) a shaped neck bridge (e.g. stent, lattice, mesh, or framework) which is inserted into the aneurysm sac, is expanded (e.g. self-expands) into a substantially ellipsoidal shape within the aneurysm sac, and then is collapsed into a double-layer, upward-opening, substantially paraboloidal shape which substantially covers the inside of the aneurysm neck; (d) a (central) opening in the neck bridge through which the coils are inserted into the aneurysm sac; and (e) a closure mechanism which closes the opening after the coils have been inserted into the aneurysm sac.

In an example, an aneurysm occlusion device can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck, wherein the expandable proximal member is a spherical stent which has been collapsed or folded into a hemispherical shape; (d) a (central) opening in the neck bridge through which the coils are inserted into the aneurysm sac; and (e) a closure mechanism which closes the opening after the coils have been inserted into the aneurysm sac.

In an example, an expandable proximal member (e.g. neck bridge) can be a multi-layer neck bridge which is made by folding and/or compressing a generally spherical or ellipsoidal stent, lattice, mesh, or framework into a multi-layer bowl or cup shaped stent, lattice, mesh, or framework. In an example, an expandable proximal member (e.g. neck bridge) can be a multi-layer neck bridge which is made by folding and/or compressing a generally spherical or ellipsoidal stent, lattice, mesh, or framework within the aneurysm sac. In an example, an expandable proximal member (e.g. neck bridge) can be a multi-layer neck bridge which is made by folding, twisting, and/or inverting a generally tubular stent, lattice, mesh, or framework into a multi-layer bowl or cup shaped stent, lattice, mesh, or framework.

In an example, an aneurysm occlusion device can comprise: (a) an expandable net or mesh which is inserted into an aneurysm sac; wherein a proximal portion of the expandable net or mesh is configured to be a first distance from the aneurysm neck after the net or mesh has been inserted into the aneurysm sac; wherein a distal portion of the expandable net or mesh is configured to be a second distance from the aneurysm neck after the net or mesh has been inserted into the aneurysm sac; wherein the second distance is greater than the first distance; wherein the proximal portion of the expandable net or mesh has a first level of flexibility, elasticity, conformability, and/or compliance; wherein the distal portion of the expandable net or mesh has a second level of flexibility, elasticity, conformability, and/or compliance; and wherein second level of flexibility, elasticity, conformability, and/or compliance is greater than the first level of flexibility, elasticity, conformability, and/or compliance; and (b) an embolic liquid which is inserted into, retained within, and congealed within the expandable net or mesh after the net or mesh has been inserted into the aneurysm sac.

In an example, an aneurysm occlusion device can comprise a proximal stent which is expanded to a globular shape within an aneurysm sac and then compressed into a bowl shape which covers the aneurysm neck. The device can further comprise embolic members and/or embolic material which is inserted into a distal portion of the aneurysm sac. The proximal stent component can cover the aneurysm neck so as to reduce blood flow into the aneurysm sac and the accumulated embolic members and/or embolic material in the distal portion of the aneurysm sac can keep the proximal stent in place.

In an example, an aneurysm occlusion device can comprise: a proximal stent, wherein the proximal stent is inserted into an aneurysm sac, expanded within the aneurysm sac into a spherical, ellipsoidal, and/or globular configuration, and then collapsed within the aneurysm sac into a hemispherical, bowl, and/or distally-concave configuration which covers the aneurysm neck; embolic members and/or material, wherein the embolic members and/or material is inserted into a distal portion of the aneurysm sac, thereby exerting pressure on the distal surface of the proximal stent and compressing the proximal stent from its spherical, ellipsoidal, and/or globular configuration to its hemispherical, bowl, and/or distally-concave configuration; and a catheter and/or other lumen, wherein the embolic members and/or material is delivered through the catheter and/or other lumen into the distal portion of the aneurysm sac.

In an example, an aneurysm occlusion device can comprise: a proximal stent, wherein the proximal stent is inserted into an aneurysm sac, expanded within the aneurysm sac into a spherical, ellipsoidal, and/or globular configuration, and then collapsed within the aneurysm sac into a hemispherical, bowl, and/or distally-concave configuration which covers the aneurysm neck;

embolic members and/or material, wherein the embolic members and/or material is inserted into a distal portion of the aneurysm sac; a catheter and/or other lumen, wherein the embolic members and/or material is delivered through the catheter and/or other lumen into the distal portion of the aneurysm sac; and a wire, cord, and/or filament, wherein the wire, cord, and/or filament is pulled to collapse the proximal stent into the hemispherical, bowl, and/or distally concave shape.

In an example, an aneurysm occlusion device can comprise: a proximal stent, wherein the proximal stent is inserted into an aneurysm sac, expanded within the aneurysm sac into a spherical, ellipsoidal, and/or globular configuration, and then collapsed within the aneurysm sac into a hemispherical, bowl, and/or distally-concave configuration which covers the aneurysm neck; a distal flexible mesh or net, wherein the flexible mesh or net is inserted into the aneurysm sac, and wherein the most distal portion of the flexible mesh or net is farther from the aneurysm neck than the most distal portion of the stent in its hemispherical, bowl, and/or distally-concave configuration; embolic members and/or material, wherein the embolic members and/or material is inserted into the flexible mesh or net, wherein insertion of the embolic members and/or material into the flexible mesh or net expands the flexible mesh or net to conform to the walls of even an irregularly-shaped aneurysm sac, and wherein insertion of the embolic members and/or material into the flexible mesh or net also helps to keep the stent in place covering the aneurysm neck; a catheter and/or other lumen, wherein the embolic members and/or material is delivered through the catheter and/or other lumen into the flexible mesh or net; and a wire, cord, and/or filament, wherein the wire, cord, and/or filament is pulled to collapse the proximal stent into the hemispherical, bowl, and/or distally concave shape.

In an example, an aneurysm occlusion device can comprise a proximal stent which is first expanded to a globular shape within an aneurysm sac and then compressed into a bowl shape which covers the aneurysm neck. The device can further comprise embolic members and/or embolic material which is inserted into a distal portion of the aneurysm sac. In an example, the proximal stent can be compressed from a globular shape to a bowl shape when an operator pulls on a wire or cord attached to a distal portion of the proximal stent.

In an example, a proximal stent can be compressed from a globular shape to a bowl shape by pressure from the accumulation of embolic members and/or embolic material inserted into the distal portion of the aneurysm sac. The proximal stent component covers the aneurysm neck so as to reduce blood flow into the aneurysm sac and the accumulated embolic members and/or embolic material in the distal portion of the aneurysm sac keep the proximal stent in place, gently pressing the proximal stent against the aneurysm neck from inside the aneurysm sac. In an example, this device can further comprise a distal flexible net or mesh into which the embolic members and/or embolic material is inserted in the distal portion of the aneurysm sac. The flexible net or mesh can reduce the possibility of embolic members and/or embolic material escaping from the aneurysm sac.

In an example, embolic members and/or material inserted into the flexible net or mesh can be liquid which congeals and/or solidifies. In an example, embolic members and/or material inserted into the flexible net or mesh can be a polymer which congeals and/or solidifies. In an example, embolic members and/or material inserted into the flexible net or mesh can be a liquid embolic material. In an example, embolic members and/or material inserted into the flexible net or mesh can be hydrogel material. In an example, embolic members and/or material inserted into the flexible net or mesh can be congealing adhesive material. In an example, accumulation of embolic members and/or material in an aneurysm sac can compress a flexible net or mesh from a spherical, ellipsoidal, and/or globular configuration to a hemispherical, bowl-shaped, and/or distally-concave configuration by pressing against the distal surface of the flexible net or mesh.

In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac. In an example, accumulation of embolic members and/or material in a space between a proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) toward a proximal portion of the proximal mesh.

In an example, accumulation of embolic members and/or material in a space between a proximal mesh (e.g. neck bridge) in an aneurysm sac and a wall (e.g. the distal wall or dome) of the aneurysm sac can push, press, and/or deform a distal portion of the proximal mesh and changing the concavity of the distal portion of the mesh (relative to a proximal portion of the proximal mesh). In an example, accumulation of embolic members and/or material in a space between a proximal mesh (e.g. neck bridge) in an aneurysm sac and a wall (e.g. the distal wall or dome) of the aneurysm sac can push, press, and/or deform a distal portion of the proximal mesh and changing the convexity of the distal portion of the mesh (relative to a proximal portion of the proximal mesh).

In an example, accumulation of embolic members and/or material in a space between a proximal mesh (e.g. neck bridge) in an aneurysm sac and a wall (e.g. the distal wall or dome) of the aneurysm sac can push, press, and/or deform a distal portion of the proximal mesh and changing the distal portion of the mesh from being convex to concave (relative to a proximal portion of the proximal mesh). In an example, accumulation of embolic members and/or material in a space between a proximal mesh (e.g. neck bridge) in an aneurysm sac and a wall (e.g. the distal wall or dome) of the aneurysm sac can push, press, and/or deform a distal portion of the proximal mesh and changing the concavity of the distal portion of the mesh from facing in a distal direction to facing in a proximal direction.

In an example, accumulation of embolic members and/or material in a space between a proximal mesh (e.g. neck bridge) in an aneurysm sac and a wall (e.g. the distal wall or dome) of the aneurysm sac can push, press, and/or deform a distal portion of the proximal mesh, thereby inverting the distal surface of the proximal mesh into the proximal surface of the proximal mesh. In an example, accumulation of embolic members and/or material in a space between a proximal mesh (e.g. neck bridge) in an aneurysm sac and a wall (e.g. the distal wall or dome) of the aneurysm sac can push, press, and/or deform a distal portion of the proximal mesh, thereby folding the distal portion of the proximal mesh into the proximal portion of the proximal mesh.

In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein the interior of proximal mesh is smaller in the second configuration than in the first configuration. In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein a distal end and/or hub of the proximal mesh connects to a proximal end and/or hub of the proximal mesh in the second configuration.

In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein the distal surface of the proximal mesh curves outward in a distal direction in the first configuration and has a bowl (e.g. hemisphere, semi-ellipsoid, inverted umbrella, or cup) shape in the second configuration.

In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein the proximal mesh has a generally-globular (e.g. sphere, ellipsoid, ovaloid, or prolate sphere) shape in the first configuration and a toroidal and/or doughnut shape in the second configuration.

In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein the proximal mesh has a half-toroidal and/or sliced-doughnut shape in the second configuration. In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein the proximal mesh has a funnel shape in the second configuration.

In an example, a proximal mesh (e.g. neck bridge) can have: a first configuration with a distal end and/or hub and a proximal end and/or hub after having been expanded in an aneurysm sac; and a second configuration after having been compressed in a distal-to-proximal direction by accumulation of embolic members and/or embolic material which has been inserted into a space between the proximal mesh and the wall (e.g. the distal wall or dome) of the aneurysm sac; wherein the distal end and/or hub connects with the proximal end and/or hub in the second configuration.

In an example, insertion of embolic members and/or material into a space between a globular (e.g. spherical, ellipsoidal, ovaloid, prolate spherical, apple, or barrel shaped) proximal mesh (e.g. neck bridge) in an aneurysm sac and a wall (e.g. the distal wall or dome) of the aneurysm sac can push, press, and/or deform a distal portion of the proximal mesh in a proximal direction, thereby causing the distal portion to invert and/or fold into multi-layer bowl (e.g. hemisphere, semi-ellipsoid, inverted umbrella, or cup) shape.

In an example, insertion of embolic members and/or material into a space between a mesh (e.g. neck bridge) with a first shape and a first level of wall flexibility in an aneurysm sac and a wall (e.g. the distal wall or dome) of the aneurysm sac can push, press, and/or deform a distal portion of the mesh in a proximal direction, thereby causing the mesh to compress, fold, and/or invert into a second shape and a second level of wall flexibility, wherein the second level of wall flexibility is less than the first level of wall flexibility.

In an example, insertion of embolic members and/or material into a space between a one-layer convex (e.g. spherical, ellipsoidal, ovaloid, prolate spherical, apple, or barrel) proximal mesh (e.g. neck bridge) in an aneurysm sac and a wall (e.g. the distal wall or dome) of the aneurysm sac can push, press, and/or deform a distal portion of the proximal mesh in a proximal direction, thereby causing the distal portion to invert and/or fold into two-layer concave (e.g. hemisphere, semi-ellipsoid, inverted umbrella, or cup) shape.

In an example, accumulation of embolic members and/or material in a space between a globular (e.g. spherical, ellipsoidal, ovaloid, prolate spherical, apple, or barrel shaped) proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) into a proximal portion of the proximal mesh and changing the proximal mesh into a multi-layer bowl (e.g. hemisphere, semi-ellipsoid, inverted umbrella, or cup) shape.

In an example, insertion of embolic members and/or material into a space between a globular (e.g. spherical, ellipsoidal, ovaloid, prolate spherical, apple, or barrel shaped) proximal mesh (e.g. neck bridge) in an aneurysm sac and a wall (e.g. the distal wall or dome) of the aneurysm sac can push, press, and/or deform a distal portion of the proximal mesh, thereby causing the distal portion to invert and/or fold into (a concavity of) a proximal portion of the proximal mesh and changing the proximal mesh into a multi-layer bowl (e.g. hemisphere, semi-ellipsoid, inverted umbrella, or cup) shape.

In an example, insertion of embolic members and/or material into a space between a globular (e.g. spherical, ellipsoidal, ovaloid, prolate spherical, apple, or barrel shaped) proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) toward a proximal portion of the proximal mesh and changing the proximal mesh into a multi-layer bowl (e.g. hemisphere, semi-ellipsoid, inverted umbrella, or cup) shape.

In an example, accumulation of embolic members and/or material in a space between a convex proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) in a proximal direction and changing the proximal mesh into a funnel shape (e.g. hyperbolic, parabolic, or frustal shape). In an example, insertion of embolic members and/or material into a space between a convex proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) into a proximal portion of the proximal mesh and changing the proximal mesh into a funnel shape (e.g. hyperbolic, parabolic, or frustal shape).

In an example, accumulation of embolic members and/or material in a space between a globular proximal mesh (e.g. neck bridge) in an aneurysm sac and a wall (e.g. the distal wall or dome) of the aneurysm sac can push and deform a distal portion of the proximal mesh in a proximal direction, thereby causing the distal portion of the globular proximal mesh to change into a funnel shape (e.g. hyperbolic, parabolic, or frustal shape) and the proximal portion of the proximal mesh to change into a bowl shape (e.g. hemispherical, semi-ellipsoidal, inverted umbrella, or cup shape).

In an example, accumulation of embolic members and/or material in a space between a convex proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, and/or invert) into a proximal portion of the proximal mesh and changing the proximal mesh into a toroidal (e.g. doughnut or bagel) shape. In an example, insertion of embolic members and/or material into a space between a convex proximal mesh (e.g. neck bridge) in an aneurysm sac and a wall (e.g. the distal wall or dome) of the aneurysm sac can push, press, and/or deform a distal portion of the proximal mesh, thereby causing the distal portion to invert into (a concavity of) a proximal portion of the proximal mesh and changing the proximal mesh into a toroidal (e.g. doughnut or bagel) shape.

In an example, insertion of embolic members and/or material into a space between a convex proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) toward a proximal portion of the proximal mesh and changing the proximal mesh into a toroidal (e.g. doughnut or bagel) shape. In an example, the interior of a proximal mesh can have a cardioid shape. In an example, the interior of a proximal mesh can have a funnel shape. In an example, the interior of a proximal mesh can have a half-hyperbolic shape. In an example, the interior of a proximal mesh can have a hyperbolic shape. In an example, the interior of a proximal mesh can have an apple shape.

In an example, a distal portion of a proximal mesh can comprise a central opening and/or aperture through which embolic members and/or embolic material pass into a space between the proximal mesh and the aneurysm wall. In an example, a distal portion of a proximal mesh can comprise a convex opening and/or aperture through which embolic members and/or embolic material pass into a space between the proximal mesh and the aneurysm wall. In an example, a distal portion of a proximal mesh can comprise an opening and/or aperture through which embolic members and/or embolic material pass into a space between the proximal mesh and the aneurysm wall, wherein this opening and/or aperture can be closed by the application of electrical energy after embolic members and/or embolic material has been inserted into this space.

In an example, a folded distal portion of a proximal mesh can form an opening and/or aperture through which embolic members and/or embolic material pass into a space between the proximal mesh and the aneurysm wall. In an example, a proximal mesh can have a distal hub, wherein there is an opening and/or lumen through this hub through which embolic members and/or embolic material is inserted into a space between the hub and the aneurysm wall.

In an example, a proximal mesh can have a distal hub; wherein there is an opening and/or lumen through this hub through which embolic members and/or embolic material is inserted into a space between the hub and the aneurysm wall; wherein this hub comprises two (inner and outer) rings, bands, or cylinders; wherein the rings, bands, or cylinders are nested and/or concentric; wherein embolic members and/or embolic material is inserted into the space through the inner ring, band, or cylinder; and wherein filaments comprising the proximal mesh are held (e.g. pinched, compressed, glued, and/or welded) between the inner and outer rings, bands, or cylinders. In an example, distal ends of filaments comprising a proximal mesh can be connected together at a distal hub, wherein embolic members and/or embolic material are inserted through this hub into a space between the hub and the wall (e.g. distal wall or dome) of an aneurysm sac.

In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck and a distal portion which is farther from the aneurysm neck, wherein the proximal portion spans between 20% and 45% of the interior of the proximal mesh. In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck and a distal portion which is farther from the aneurysm neck, wherein the proximal portion spans between 45% and 55% of the interior of the proximal mesh.

In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck and a distal portion which is farther from the aneurysm neck, wherein the distal portion spans between 20% and 45% of the interior of the proximal mesh. In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck and a distal portion which is farther from the aneurysm neck, wherein the distal portion spans between 45% and 55% of the interior of the proximal mesh.

In an example, accumulation of embolic members and/or material in a space between a proximal mesh (e.g. neck bridge) in an aneurysm sac and a wall (e.g. the distal wall or dome) of the aneurysm sac can push and/or force a distal half of the proximal mesh and increasing the concavity of the distal half of the mesh (relative to a proximal half of the proximal mesh). In an example, accumulation of embolic members and/or material in a space between a proximal mesh (e.g. neck bridge) in an aneurysm sac and a wall (e.g. the distal wall or dome) of the aneurysm sac can push and/or force a distal half of the proximal mesh and increasing the convexity of the distal half of the mesh (relative to a proximal half of the proximal mesh).

In an example, accumulation of embolic members and/or material in a space between a proximal mesh (e.g. neck bridge) in an aneurysm sac and a wall (e.g. the distal wall or dome) of the aneurysm sac can push and/or force a distal half of the proximal mesh and changing the distal half of the mesh from being concave to convex (relative to a proximal half of the proximal mesh). In an example, accumulation of embolic members and/or material in a space between a proximal mesh (e.g. neck bridge) in an aneurysm sac and a wall (e.g. the distal wall or dome) of the aneurysm sac can push and/or force a distal half of the proximal mesh, thereby causing the distal half to invert and/or fold into (a concavity of) a proximal half of the proximal mesh. In an example, insertion of embolic members and/or material into a space between a proximal mesh (e.g. neck bridge) in an aneurysm sac and a wall (e.g. the distal wall or dome) of the aneurysm sac can push and/or force a distal half of the proximal mesh, thereby causing the distal half to move (e.g. bend, curve, deform, fold, and/or invert) toward a proximal half of the proximal mesh.

In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein a distal third of the proximal mesh is closer to a proximal third of the proximal mesh in the second configuration than in the first configuration.

In an example, accumulation of embolic members and/or material in a space between a proximal mesh (e.g. neck bridge) in an aneurysm sac and a wall (e.g. the distal wall or dome) of the aneurysm sac can push and/or force a distal third of the proximal mesh, thereby causing the distal third to move (e.g. bend, curve, deform, fold, and/or invert) in a proximal direction. In an example, insertion of embolic members and/or material into a space between a proximal mesh (e.g. neck bridge) in an aneurysm sac and a wall (e.g. the distal wall or dome) of the aneurysm sac can push and/or force a distal third of the proximal mesh, thereby causing the distal third to move (e.g. bend, curve, deform, fold, and/or invert) into a proximal third of the proximal mesh.

In an example, embolic material of this device can be a bioactive polymer material. In an example, embolic material of this device can be a liquid embolic material. In an example, embolic members of this device can be embolic ribbons. In an example, embolic members of this device can be microsponges. In an example, embolic members of this device can comprise metal embolic coils. In an example, embolic members of this device can comprise polymer strands. In an example, embolic members of this device can comprise solid or hollow polymer noodles.

In an example, a method for occluding an aneurysm can comprise: inserting a proximal mesh (e.g. neck bridge) into an aneurysm sac; expanding the proximal mesh into a convex (e.g. spherical, ellipsoidal, ovaloid, prolate spheroid, and/or generally globular) shape; inserting a catheter through the interior of the proximal mesh into a space between the proximal mesh and the wall (e.g. distal wall or dome) of the aneurysm; and inserting embolic members and/or embolic material through the catheter into this space, wherein accumulation of the embolic member and/or embolic material in this space compresses the distal surface of the proximal mesh in a proximal direction, thereby deforming, folding, and/or inverting the proximal mesh into a bowl shape (e.g. hemispherical, semi-ellipsoidal, inverted umbrella shape, or cup shape).

In an example, this device can comprise a first catheter and/or lumen through which an proximal mesh is delivered into an aneurysm sac and a second catheter and/or lumen through which embolic members and/or material is delivered to a space between the proximal mesh and the wall (e.g. distal wall or dome) of the aneurysm sac, wherein the second catheter and/or lumen passes through a non-central opening in the proximal mesh (e.g. neck bridge). In an example, this device can comprise a first catheter and/or lumen through which an proximal mesh is delivered into an aneurysm sac and a second catheter and/or lumen through which embolic members and/or material is delivered to a space between the proximal mesh and the wall (e.g. distal wall or dome) of the aneurysm sac, wherein the second catheter and/or lumen is detachably-connected to a central opening in the proximal mesh (e.g. neck bridge).

In an example, this device can comprise a first catheter and/or lumen through which an proximal mesh is delivered into an aneurysm sac and a second catheter and/or lumen through which embolic members and/or material is delivered to a space between the proximal mesh and the wall (e.g. distal wall or dome) of the aneurysm sac, wherein the first and second catheter and/or lumens are inserted sequentially into the aneurysm sac. In an example, this device can comprise a first catheter and/or lumen through which an proximal mesh is delivered into an aneurysm sac and a second catheter and/or lumen through which embolic members and/or material is delivered to a space between the proximal mesh and the wall (e.g. distal wall or dome) of the aneurysm sac, wherein the first and second catheter and/or lumens are parallel to each other.

In an example, this device can comprise a first catheter and/or lumen through which an proximal mesh is delivered into an aneurysm sac and a second catheter and/or lumen through which embolic members and/or material is delivered to a space between the proximal mesh and the wall (e.g. distal wall or dome) of the aneurysm sac, wherein the first catheter and/or lumen passes through a non-central opening in the proximal mesh (e.g. neck bridge). In an example, this device can comprise a first catheter and/or lumen through which an proximal mesh is delivered into an aneurysm sac and a second catheter and/or lumen through which embolic members and/or material is delivered to a space between the proximal mesh and the wall (e.g. distal wall or dome) of the aneurysm sac, wherein the second catheter and/or lumen slides through a hub and/or opening in the proximal surface of the proximal mesh (e.g. neck bridge).

In an example, this device can comprise a first catheter and/or lumen through which a proximal mesh (e.g. neck bridge) is delivered into an aneurysm sac and a second catheter and/or lumen through which embolic members and/or material is delivered to a space between the proximal mesh and the wall (e.g. distal wall or dome) of the aneurysm sac, wherein the second catheter and/or lumen has a larger diameter than the first catheter and/or lumen. In an example, this device can comprise a first catheter and/or lumen through which a proximal mesh (e.g. neck bridge) is delivered into an aneurysm sac and a second catheter and/or lumen through which embolic members and/or material is delivered to a space between the proximal mesh and the wall (e.g. distal wall or dome) of the aneurysm sac, wherein the second catheter and/or lumen extends farther into the aneurysm sac than the first catheter and/or lumen.

In an example, a method for occluding an aneurysm can comprise: inserting a proximal mesh (e.g. neck bridge) into an aneurysm sac; expanding the proximal mesh into a first shape, wherein this first shape is convex; inserting a catheter through the proximal mesh into a space between the proximal mesh and the wall (e.g. distal wall or dome) of the aneurysm sac; inserting embolic members and/or embolic material into this space, wherein accumulation of the embolic members and/or embolic material compresses, folds, and/or inverts a distal portion of the proximal mesh into a proximal portion of the proximal mesh, wherein this compression, folding, and/or inversion changes the proximal mesh into a second shape, wherein this second shape is a bowl (e.g. hemisphere, semi-ellipsoid, inverted umbrella, or cup) shape; and withdrawing the catheter from the proximal mesh and the aneurysm sac.

In an example, a method for occluding an aneurysm can comprise: inserting a proximal mesh (e.g. neck bridge) into an aneurysm sac; expanding the proximal mesh into a convex (e.g. spherical, ellipsoidal, ovaloid, prolate spheroid, and/or generally globular) shape; and inserting embolic members and/or embolic material through an opening in the proximal mesh into space between the proximal mesh and the wall (e.g. distal wall or dome) of the aneurysm sac, wherein accumulation of the embolic member and/or embolic material in this space compresses the distal surface of the proximal mesh in a proximal direction, thereby deforming, folding, and/or inverting the proximal mesh into a bowl shape (e.g. hemispherical, semi-ellipsoidal, inverted umbrella shape, or cup shape).

In an example, a central inversion of a convex proximal mesh can form a lumen through the proximal mesh, wherein embolic members and/or embolic material is inserted through this lumen into a space between the proximal mesh and the wall (e.g. distal wall or dome) of an aneurysm sac. In an example, a central inversion of the distal half a globular proximal mesh can form a lumen through the proximal mesh, wherein embolic members and/or embolic material is inserted through this lumen into a space between the proximal mesh and the wall (e.g. distal wall or dome) of an aneurysm sac. In an example, a device can further comprise a central pathway (e.g. an opening, catheter, tube, or mesh column) through a toroidal (e.g. doughnut or bagel shaped) proximal mesh, wherein embolic members and/or embolic material is inserted through this pathway into a space between the proximal mesh and the wall (e.g. distal wall or dome) of an aneurysm sac.

In an example, a device can further comprise a funnel-shaped pathway (e.g. a catheter, tube, or mesh column) through the interior of a proximal mesh along a central proximal-to-distal axis of the proximal mesh, wherein embolic members and/or embolic material is inserted through this pathway into a space between the proximal mesh and the wall (e.g. distal wall or dome) of an aneurysm sac. In an example, a method for occluding an aneurysm can comprise: inserting a proximal mesh (e.g. neck bridge) into an aneurysm sac; expanding the proximal mesh into a convex (e.g. spherical, ellipsoidal, ovaloid, prolate spheroid, and/or generally globular) shape; and inserting embolic members and/or embolic material through a central column and/or lumen in the proximal mesh into space between the proximal mesh and the wall (e.g. distal wall or dome) of the aneurysm sac, wherein accumulation of the embolic member and/or embolic material in this space compresses the distal surface of the proximal mesh in a proximal direction, thereby deforming, folding, and/or inverting the proximal mesh into a bowl shape (e.g. hemispherical, semi-ellipsoidal, inverted umbrella shape, or cup shape).

In an example, this device can further comprise a closure mechanism which closes an opening and/or pathway through a proximal mesh after embolic members and/or embolic material has been inserted through the opening and/or pathway, wherein this closure mechanism is remotely activated by a person operating the device by the person pulling or rotating a flexible longitudinal member (e.g. wire, suture, string, thread, or tube). In an example, this device can further comprise a closure mechanism which closes an opening and/or pathway through a proximal mesh after embolic members and/or embolic material has been inserted through the opening and/or pathway, wherein this closure mechanism is a valve.

In an example, this device can further comprise a closure mechanism which closes an opening and/or pathway through a proximal mesh after embolic members and/or embolic material has been inserted through the opening and/or pathway, wherein this closure mechanism is a plug. In an example, this device can further comprise a closure mechanism which closes an opening and/or pathway through a proximal mesh after embolic members and/or embolic material has been inserted through the opening and/or pathway, wherein this closure mechanism is an elastic loop or band.

In an example, a proximal mesh (e.g. neck bridge) can comprise a proximal portion, a distal portion, and a weaker portion between the proximal and distal portions, wherein the weaker portion is more elastic and/or more flexible than the proximal and distal portions, thereby facilitating folding and/or inversion of the distal portion into the proximal portion along this transition portion when embolic members and/or embolic material pushes on the distal portion. In an example, a proximal mesh (e.g. neck bridge) can have a convex (e.g. globular) shape when it is first expanded within an aneurysm sac, wherein the proximal mesh further comprises a weaker circumferential portion (e.g. equatorial band) with greater flexibility, greater elasticity, greater compliance, and/or thinner width than the rest of the proximal mesh, wherein this encourages the distal surface of the proximal mesh to deform, invert, and/or fold along this circumferential portion when it is pushed by the accumulation of embolic members and/or material between the proximal mesh and the wall (e.g. distal wall or dome) of the aneurysm sac.

In an example, a proximal mesh (e.g. neck bridge) can have a convex (e.g. globular) shape when it is first expanded within an aneurysm sac, wherein the proximal mesh further comprises a circumferential portion (e.g. equatorial band) which is weakened by the selective application of electrical energy to this portion by the device operator after expansion of the proximal mesh, wherein this weakening facilitates deformation, inversion, and/or folding of the proximal mesh along this circumferential portion. In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck, a distal portion which is farther from the aneurysm neck, and an intermediate portion (e.g. a circumferential or equatorial band) between the proximal and distal portions, wherein the intermediate portion is more flexible, more elastic, less dense, thinner, and/or weaker than the proximal and distal portions, and wherein the intermediate portion spans between 5% and 15% of the proximal-to-distal axis of the proximal mesh.

In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck, a distal portion which is farther from the aneurysm neck, and an intermediate portion (e.g. a circumferential or equatorial band) between the proximal and distal portions, wherein the intermediate portion is more flexible, more elastic, less dense, thinner, and/or weaker than the proximal and distal portions, and wherein the intermediate portion can be selectively weakened by the application of electrical energy.

In an example, a proximal mesh (e.g. neck bridge) can be made by braiding and/or weaving filaments, wires, and/or polymer strands together. In an example, a proximal mesh can be made with braided or woven longitudinal members (e.g. filaments, wires, tubes, or strands); wherein the proximal mesh has a first configuration after it has been expanded in an aneurysm sac, but before a distal portion of the proximal mesh has been moved in a proximal direction; wherein the proximal mesh has a second configuration after a distal portion of the proximal mesh has been moved in a proximal direction; wherein first ends of the longitudinal members converge (e.g. meet or are attached together) at a proximal hub in the first configuration; wherein second ends of the longitudinal members converge (e.g. meet or are attached together) at a distal hub in the first configuration; and wherein the distal hub contacts the proximal hub in the second configuration.

In an example, a proximal mesh can be made with braided or woven longitudinal members (e.g. filaments, wires, tubes, or strands), wherein the proximal mesh has a first configuration after it has been expanded in an aneurysm sac, but before a distal portion of the proximal mesh has been moved in a proximal direction, wherein the proximal mesh has a second configuration after a distal portion of the proximal mesh has been moved in a proximal direction, wherein first ends of the longitudinal members meet at a proximal hub and second ends of the longitudinal members meet at a distal hub when the proximal mesh is in the first configuration, and wherein both ends meet at a proximal hub when the proximal mesh is in the second configuration.

In an example, a proximal mesh can be made with braided or woven longitudinal members (e.g. filaments, wires, tubes, or strands); wherein the proximal mesh has a first configuration after it has been expanded in an aneurysm sac, but before a distal portion of the proximal mesh has been moved in a proximal direction; wherein the proximal mesh has a second configuration after a distal portion of the proximal mesh has been moved in a proximal direction; wherein first ends of the longitudinal members converge (e.g. meet and/or are attached together) in the proximal half of the proximal mesh in the first configuration; wherein second ends of the longitudinal members converge (e.g. meet and/or are attached together) in the distal half of the proximal mesh in the first configuration; wherein first and second ends of the longitudinal members converge (e.g. meet and/or are attached together) in the proximal half of the proximal mesh in the second configuration; and wherein the distal half of the proximal mesh includes folds of the longitudinal members in the second configuration.

In an example, a proximal mesh can be made with braided or woven longitudinal members (e.g. filaments, wires, tubes, or strands); wherein the proximal mesh has a first configuration after it has been expanded in an aneurysm sac, but before a distal portion of the proximal mesh has been moved in a proximal direction; wherein the proximal mesh has a second configuration after a distal portion of the proximal mesh has been moved in a proximal direction; wherein first ends of the longitudinal members converge (e.g. meet and/or are attached together) in a proximal location of the proximal mesh in the first configuration; wherein second ends of the longitudinal members converge (e.g. meet and/or are attached together) in a distal location of the proximal mesh in the first configuration; wherein first and second ends of the longitudinal members converge (e.g. meet and/or are attached together) in a proximal location of the proximal mesh in the second configuration; and wherein the distal surface of the proximal mesh comprises folds in the longitudinal members in the second configuration. In an example, a proximal mesh (e.g. neck bridge) can be positioned over an aneurysm neck after it has been expanded within an aneurysm sac and before it has been proximally compressed.

In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby folding the distal surface of the proximal mesh into the proximal surface of the proximal mesh. In an alternative example, pulling or rotating a flexible longitudinal member (e.g. wire, suture, or string) which is (detachably) connected to a distal portion (e.g. distal hub) of a proximal mesh can change the convexity of the distal portion of the mesh (relative to a proximal portion of the proximal mesh).

In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to invert and/or fold into (a concavity of) a proximal portion of the proximal mesh and changing the proximal mesh into a multi-layer bowl (e.g. hemisphere, semi-ellipsoid, inverted umbrella, or cup) shape. In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to invert and/or fold into a concavity of a proximal portion of the proximal mesh and changing the proximal mesh into a multi-layer bowl (e.g. hemisphere, semi-ellipsoid, inverted umbrella, or cup) shape.

In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to move (e.g. bend, curve, deform, fold, and/or invert) toward a proximal portion of the proximal mesh and changing the proximal mesh into a funnel shape (e.g. hyperbolic, parabolic, or frustal shape).

In an alternative example, a method for occluding an aneurysm can comprise: inserting a proximal mesh (e.g. neck bridge) into an aneurysm sac and expanding the proximal mesh into a generally-globular shape (e.g. sphere, ellipsoid, oval, or prolate sphere shape); pulling in a proximal direction a wire, string, suture, filament, or other flexible longitudinal member which is attached to a distal portion of the proximal mesh, thereby compressing, folding, and/or inverting the distal portion of the proximal mesh into a proximal portion of the proximal mesh and changing the proximal mesh into a bowl (e.g. hemisphere, semi-ellipsoid, inverted umbrella, or cup) shape; and then inserting embolic members and/or embolic material into a space between a distal portion of the proximal member and the wall (e.g. distal wall or dome) of the aneurysm sac.

In an alternative example, a method for occluding an aneurysm can comprise: inserting a proximal mesh (e.g. neck bridge) into an aneurysm sac and expanding the proximal mesh into a generally-globular shape (e.g. sphere, ellipsoid, oval, or prolate sphere shape); pulling in a proximal direction a wire, string, suture, filament, or other flexible longitudinal member which is attached to a distal portion of the proximal mesh, thereby compressing, folding, and/or inverting the distal portion of the proximal mesh into a proximal portion of the proximal mesh and changing the proximal mesh into a bowl (e.g. hemisphere, semi-ellipsoid, inverted umbrella, or cup) shape; and then inserting embolic members and/or embolic material through the proximal mesh between the proximal mesh and the wall (e.g. distal wall or dome) of the aneurysm sac.

In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction. In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to move (e.g. bend, curve, deform, fold, and/or invert) in a proximal direction, thereby changing the proximal mesh into a toroidal (e.g. doughnut or bagel) shape.

In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to move (e.g. bend, curve, deform, and/or invert) into a proximal portion of the proximal mesh and changing the proximal mesh into a toroidal (e.g. doughnut or bagel) shape.

In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion of the globular proximal mesh to form a half-toroidal (e.g. sliced bagel) shape and the proximal portion of the proximal mesh to form a bowl shape (e.g. hemispherical, semi-ellipsoidal, inverted umbrella, or cup shape).

In an alternative example, a proximal mesh (e.g. neck bridge) can have a first configuration after having been expanded in an aneurysm sac and a subsequent second configuration after a flexible longitudinal member (e.g. wire, suture, or string) which is (detachably) connected to a distal portion (e.g. distal hub) of the proximal mesh has been pulled or rotated, wherein a distal end and/or hub of the proximal mesh does not contact the proximal end and/or hub of the proximal mesh in the first configuration, but does contact the proximal end/or hub in the second configuration.

In an alternative example, a proximal mesh (e.g. neck bridge) can have a first configuration after having been expanded in an aneurysm sac and a subsequent second configuration after a flexible longitudinal member (e.g. wire, suture, or string) which is (detachably) connected to a distal portion (e.g. distal hub) of the proximal mesh has been pulled or rotated, wherein a distal surface of the proximal mesh is distally-convex in the first configuration and has a bowl (e.g. hemisphere, semi-ellipsoid, inverted umbrella, or cup) shape (curving in a proximal direction) in the second configuration.

In an alternative example, a proximal mesh (e.g. neck bridge) can have a first configuration after having been expanded in an aneurysm sac and a subsequent second configuration after a flexible longitudinal member (e.g. wire, suture, or string) which is (detachably) connected to a distal portion (e.g. distal hub) of the proximal mesh has been pulled or rotated, wherein the proximal mesh has a toroidal and/or doughnut shape in the second configuration. In an alternative example, a proximal mesh (e.g. neck bridge) can have a first configuration after having been expanded in an aneurysm sac and a subsequent second configuration after a flexible longitudinal member (e.g. wire, suture, or string) which is (detachably) connected to a distal portion (e.g. distal hub) of the proximal mesh has been pulled or rotated, wherein the proximal mesh has a funnel shape in the second configuration. In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby decreasing the flexibility of the proximal portion of the proximal mesh.

In an alternative example, a proximal mesh (e.g. neck bridge) which is inserted into an aneurysm sac—after it has been expanded in the aneurysm sac but before it has been compressed by pulling or rotating a flexible longitudinal member (e.g. wire, suture, or string)—can have a proximal portion which is closer to the aneurysm neck and a distal portion which is farther from the aneurysm neck, wherein the distal portion comprises between 20% and 45% of the proximal mesh. In an alternative example, a proximal mesh (e.g. neck bridge) which is inserted into an aneurysm sac—after it has been expanded in the aneurysm sac but before it has been compressed by pulling or rotating a flexible longitudinal member (e.g. wire, suture, or string)—can have a proximal portion which is closer to the aneurysm neck and a distal portion which is farther from the aneurysm neck, wherein the distal portion spans between 45% and 55% of the proximal-to-distal axis of the proximal mesh.

In an alternative example, a proximal mesh (e.g. neck bridge) can comprise a proximal portion, a distal portion, and a weaker portion between the proximal and distal portions, wherein the proximal portion comprises between 30% and 45% of the proximal mesh, wherein the distal portion comprises between 30% and 45% of the proximal mesh, wherein the weaker portion comprises between 5% and 25% of the proximal mesh, and wherein the weaker portion is more elastic and/or more flexible than the proximal and distal portions, thereby facilitating folding and/or inversion of the distal portion into the proximal portion along this transition portion when a flexible longitudinal member (e.g. wire, suture, or string) attached to the proximal mesh is pulled or rotated.

In an alternative example, a proximal mesh (e.g. neck bridge) which is inserted into an aneurysm sac—after it has been expanded in the aneurysm sac but before it has been compressed by pulling or rotating a flexible longitudinal member (e.g. wire, suture, or string)—can have a proximal portion which is closer to the aneurysm neck, a distal portion which is farther from the aneurysm neck, and an intermediate portion (e.g. a circumferential or equatorial band) between the proximal and distal portions, wherein the intermediate portion is more flexible, more elastic, less dense, thinner, and/or weaker than the proximal and distal portions, and wherein the intermediate portion spans less than 10% of the proximal-to-distal axis of the proximal mesh.

In an alternative example, a proximal mesh (e.g. neck bridge) which is inserted into an aneurysm sac—after it has been expanded in the aneurysm sac but before it has been compressed by pulling or rotating a flexible longitudinal member (e.g. wire, suture, or string)—can have a proximal portion which is closer to the aneurysm neck, a distal portion which is farther from the aneurysm neck, and an intermediate portion (e.g. a circumferential or equatorial band) between the proximal and distal portions, wherein the intermediate portion is more flexible, more elastic, less dense, thinner, and/or weaker than the proximal and distal portions, and wherein the intermediate portion spans between 15% and 35% of the proximal-to-distal axis of the proximal mesh.

In an alternative example, a proximal mesh (e.g. neck bridge) which is inserted into an aneurysm sac—after it has been expanded in the aneurysm sac but before it has been compressed by pulling or rotating a flexible longitudinal member (e.g. wire, suture, or string)—can have a proximal portion which is closer to the aneurysm neck, a distal portion which is farther from the aneurysm neck, and an intermediate portion (e.g. a circumferential or equatorial band) between the proximal and distal portions, wherein the intermediate portion is more flexible, more elastic, less dense, thinner, and/or weaker than the proximal and distal portions, and wherein the intermediate portion can be selectively and remotely weakened by the operator of the device by the application of electrical energy to facilitate movement (e.g. distortion, folding, and/or inversion) of the distal portion toward the proximal portion.

In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein a distal portion of the proximal mesh is closer to a proximal portion of the proximal mesh in the second configuration than in the first configuration. In an example, insertion of embolic members and/or material into a space between a proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) in a proximal direction.

In an example, accumulation of embolic members and/or material in a space between a proximal mesh (e.g. neck bridge) in an aneurysm sac and a wall (e.g. the distal wall or dome) of the aneurysm sac can push, press, and/or deform a distal portion of the proximal mesh and increasing the concavity of the distal portion of the mesh (relative to a proximal portion of the proximal mesh). In an example, accumulation of embolic members and/or material in a space between a proximal mesh (e.g. neck bridge) in an aneurysm sac and a wall (e.g. the distal wall or dome) of the aneurysm sac can push, press, and/or deform a distal portion of the proximal mesh and increasing the convexity of the distal portion of the mesh (relative to a proximal portion of the proximal mesh). In an example, accumulation of embolic members and/or material in a space between a proximal mesh (e.g. neck bridge) in an aneurysm sac and a wall (e.g. the distal wall or dome) of the aneurysm sac can push, press, and/or deform a distal portion of the proximal mesh and changing the distal portion of the mesh from being concave to convex (relative to a proximal portion of the proximal mesh).

In an example, accumulation of embolic members and/or material in a space between a proximal mesh (e.g. neck bridge) in an aneurysm sac and a wall (e.g. the distal wall or dome) of the aneurysm sac can push, press, and/or deform a distal portion of the proximal mesh, thereby causing the distal portion to invert and/or fold into (a concavity of) a proximal portion of the proximal mesh. In an example, accumulation of embolic members and/or material in a space between a proximal mesh (e.g. neck bridge) in an aneurysm sac and a wall (e.g. the distal wall or dome) of the aneurysm sac can push, press, and/or deform a distal portion of the proximal mesh, thereby folding the distal surface of the proximal mesh into the proximal surface of the proximal mesh. In an example, insertion of embolic members and/or material into a space between a proximal mesh (e.g. neck bridge) in an aneurysm sac and a wall (e.g. the distal wall or dome) of the aneurysm sac can push, press, and/or deform a distal portion of the proximal mesh, thereby causing the distal portion to invert and/or fold into (a concavity of) a proximal portion of the proximal mesh.

In an example, a proximal mesh (e.g. neck bridge) can have a first configuration after having been expanded in an aneurysm sac and subsequent a second configuration after having been compressed in a distal-to-proximal direction by accumulation of embolic members and/or embolic material which has been inserted into a space between the proximal mesh and the wall (e.g. the distal wall or dome) of the aneurysm sac, wherein a distal end and/or hub of the proximal mesh is closer to a proximal end and/or hub of the proximal mesh in the second configuration than in the first configuration.

In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein the distal surface of the proximal mesh is convex in the first configuration and has a funnel shape in the second configuration. In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein the distal surface of the proximal mesh is distally-convex in the first configuration and has a funnel shape (curving in a proximal direction) in the second configuration.

In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein the proximal mesh has a generally-globular (e.g. sphere, ellipsoid, ovaloid, or prolate sphere) shape in the first configuration and a half-toroidal and/or sliced-doughnut shape in the second configuration.

In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein the proximal mesh has a bowl (e.g. hemisphere, semi-ellipsoid, inverted umbrella, or cup) shape in the second configuration. In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein the proximal mesh has a funnel shape in the second configuration.

In an example, insertion of embolic members and/or material into a space between a one-layer globular (e.g. spherical, ellipsoidal, ovaloid, prolate spherical, apple, or barrel shaped) proximal mesh (e.g. neck bridge) in an aneurysm sac and a wall (e.g. the distal wall or dome) of the aneurysm sac can push, press, and/or deform a distal portion of the proximal mesh in a proximal direction, thereby causing the distal portion to invert and/or fold into two-layer bowl (e.g. hemisphere, semi-ellipsoid, inverted umbrella, or cup) shape. In an example, insertion of embolic members and/or material into a space between a mesh (e.g. neck bridge) with a first shape and a first number of layers in an aneurysm sac and a wall (e.g. the distal wall or dome) of the aneurysm sac can push, press, and/or deform a distal portion of the mesh in a proximal direction, thereby causing the mesh to compress, fold, and/or invert into a second shape with a second number of layers, wherein the second number is greater than the first number.

In an example, insertion of embolic members and/or material into a space between a mesh (e.g. neck bridge) with a first shape and a first level of wall elasticity in an aneurysm sac and a wall (e.g. the distal wall or dome) of the aneurysm sac can push, press, and/or deform a distal portion of the mesh in a proximal direction, thereby causing the mesh to compress, fold, and/or invert into a second shape and a second level of wall elasticity, wherein the second level of wall elasticity is less than the first level of wall elasticity.

In an example, a method for occluding an aneurysm can comprise: inserting a proximal mesh (e.g. neck bridge) into an aneurysm sac; expanding the proximal mesh into a convex (e.g. spherical, ellipsoidal, ovaloid, prolate spheroid, and/or generally globular) shape; and inserting embolic members and/or embolic material into space between the proximal mesh and the wall (e.g. distal wall or dome) of the aneurysm sac, wherein accumulation of the embolic member and/or embolic material in this space compresses the distal surface of the proximal mesh in a proximal direction, thereby deforming, folding, and/or inverting the proximal mesh into a bowl shape (e.g. hemispherical, semi-ellipsoidal, inverted umbrella shape, or cup shape).

In an example, accumulation of embolic members and/or material in a space between a globular (e.g. spherical, ellipsoidal, ovaloid, prolate spherical, apple, or barrel shaped) proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) in a proximal direction and changing the proximal mesh into a multi-layer bowl (e.g. hemisphere, semi-ellipsoid, inverted umbrella, or cup) shape.

In an example, insertion of embolic members and/or material into a space between a globular (e.g. spherical, ellipsoidal, ovaloid, prolate spherical, apple, or barrel shaped) proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) into a proximal portion of the proximal mesh and changing the proximal mesh into a multi-layer bowl (e.g. hemisphere, semi-ellipsoid, inverted umbrella, or cup) shape.

In an example, accumulation of embolic members and/or material in a space between a convex proximal mesh (e.g. neck bridge) in an aneurysm sac and a wall (e.g. the distal wall or dome) of the aneurysm sac can push, press, and/or deform a distal portion of the proximal mesh, thereby causing the distal portion to deform into (a concavity of) a proximal portion of the proximal mesh and changing the proximal mesh into a funnel shape (e.g. hyperbolic, parabolic, or frustal shape). In an example, accumulation of embolic members and/or material in a space between a convex proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) toward a proximal portion of the proximal mesh and changing the proximal mesh into a funnel shape (e.g. hyperbolic, parabolic, or frustal shape).

In an example, insertion of embolic members and/or material into a space between a convex proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) in a proximal direction and changing the proximal mesh into a funnel shape (e.g. hyperbolic, parabolic, or frustal shape). In an example, accumulation of embolic members and/or material in a space between a globular proximal mesh (e.g. neck bridge) in an aneurysm sac and a wall (e.g. the distal wall or dome) of the aneurysm sac can push and deform a distal portion of the proximal mesh in a proximal direction, thereby causing the distal portion of the globular proximal mesh to form a funnel shape (e.g. hyperbolic, parabolic, or frustal shape) and the proximal portion of the proximal mesh to form a bowl shape (e.g. hemispherical, semi-ellipsoidal, inverted umbrella, or cup shape).

In an example, accumulation of embolic members and/or material in a space between a convex proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) in a proximal direction, thereby changing the proximal mesh into a toroidal (e.g. doughnut or bagel) shape. In an example, insertion of embolic members and/or material into a space between a convex proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, and/or invert) into a proximal portion of the proximal mesh and changing the proximal mesh into a toroidal (e.g. doughnut or bagel) shape.

In an example, accumulation of embolic members and/or material in a space between a globular proximal mesh (e.g. neck bridge) in an aneurysm sac and a wall (e.g. the distal wall or dome) of the aneurysm sac can push and deform a distal portion of the proximal mesh in a proximal direction, thereby causing the distal portion of the globular proximal mesh to form a half-toroidal (e.g. sliced bagel) shape and the proximal portion of the proximal mesh to form a bowl shape (e.g. hemispherical, semi-ellipsoidal, inverted umbrella, or cup shape). In an example, the interior of a proximal mesh can have a conic section shape. In an example, the interior of a proximal mesh can have a generally-globular shape. In an example, the interior of a proximal mesh can have a half-parabolic shape. In an example, the interior of a proximal mesh can have a parabolic shape. In an example, the interior of a proximal mesh can have an egg shape.

In an example, a distal portion of a proximal mesh can comprise an opening and/or aperture which is centered on the proximal-to-longitudinal axis of the proximal mesh, wherein embolic members and/or embolic material pass through this opening and/or aperture into a space between the proximal mesh and the aneurysm wall. In an example, a distal portion of a proximal mesh can comprise a concave opening and/or aperture through which embolic members and/or embolic material pass into a space between the proximal mesh and the aneurysm wall. In an example, a distal portion of a proximal mesh can comprise an opening and/or aperture through which embolic members and/or embolic material pass into a space between the proximal mesh and the aneurysm wall, wherein this opening and/or aperture can be closed by insertion of a plug after embolic members and/or embolic material has been inserted into this space.

In an example, a folded distal portion of a proximal mesh can form a funnel-shaped opening and/or aperture through which embolic members and/or embolic material pass into a space between the proximal mesh and the aneurysm wall. In an example, a proximal mesh can have a distal hub; wherein there is an opening and/or lumen through this hub through which embolic members and/or embolic material is inserted into a space between the hub and the aneurysm wall; wherein this hub comprises two (inner and outer) rings, bands, or cylinders; wherein the rings, bands, or cylinders are nested and/or concentric; and wherein embolic members and/or embolic material is inserted into the space through the inner ring, band, or cylinder. In an example, distal ends of filaments comprising a proximal mesh can be connected together at a distal hub, wherein this distal hub is moved in a proximal direction by insertion of embolic members and/or embolic material into a space between the hub and the wall (e.g. distal wall or dome) of an aneurysm sac.

In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck and a distal portion which is farther from the aneurysm neck, wherein the proximal portion comprises between 20% and 45% of the proximal mesh. In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck and a distal portion which is farther from the aneurysm neck, wherein the proximal portion comprises between 45% and 55% of the proximal mesh.

In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck and a distal portion which is farther from the aneurysm neck, wherein the distal portion comprises between 20% and 45% of the proximal mesh. In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck and a distal portion which is farther from the aneurysm neck, wherein the distal portion comprises between 45% and 55% of the proximal mesh.

In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein a distal half of the proximal mesh is closer to a proximal half of the proximal mesh in the second configuration than in the first configuration. In an example, accumulation of embolic members and/or material in a space between a proximal mesh (e.g. neck bridge) in an aneurysm sac and a wall (e.g. the distal wall or dome) of the aneurysm sac can push and/or force a distal half of the proximal mesh and decreasing the concavity of the distal half of the mesh (relative to a proximal half of the proximal mesh).

In an example, accumulation of embolic members and/or material in a space between a proximal mesh (e.g. neck bridge) in an aneurysm sac and a wall (e.g. the distal wall or dome) of the aneurysm sac can push and/or force a distal half of the proximal mesh and decreasing the convexity of the distal half of the mesh (relative to a proximal half of the proximal mesh). In an example, accumulation of embolic members and/or material in a space between a proximal mesh (e.g. neck bridge) in an aneurysm sac and a wall (e.g. the distal wall or dome) of the aneurysm sac can push and/or force a distal half of the proximal mesh, thereby causing the distal half to move (e.g. bend, curve, deform, fold, and/or invert) in a proximal direction.

In an example, accumulation of embolic members and/or material in a space between a proximal mesh (e.g. neck bridge) in an aneurysm sac and a wall (e.g. the distal wall or dome) of the aneurysm sac can push and/or force a distal half of the proximal mesh, thereby causing the distal half to move (e.g. bend, curve, deform, fold, and/or invert) into a proximal half of the proximal mesh. In an example, insertion of embolic members and/or material into a space between a proximal mesh (e.g. neck bridge) in an aneurysm sac and a wall (e.g. the distal wall or dome) of the aneurysm sac can push and/or force a distal half of the proximal mesh, thereby causing the distal half to invert and/or fold into (a concavity of) a proximal half of the proximal mesh.

In an example, accumulation of embolic members and/or material in a space between a proximal mesh (e.g. neck bridge) in an aneurysm sac and a wall (e.g. the distal wall or dome) of the aneurysm sac can push and/or force a distal third of the proximal mesh, thereby causing the distal third to invert and/or fold into (a concavity of) a proximal third of the proximal mesh. In an example, accumulation of embolic members and/or material in a space between a proximal mesh (e.g. neck bridge) in an aneurysm sac and a wall (e.g. the distal wall or dome) of the aneurysm sac can push and/or force a distal third of the proximal mesh, thereby causing the distal third to move (e.g. bend, curve, deform, fold, and/or invert) toward a proximal third of the proximal mesh. In an example, insertion of embolic members and/or material into a space between a proximal mesh (e.g. neck bridge) in an aneurysm sac and a wall (e.g. the distal wall or dome) of the aneurysm sac can push and/or force a distal third of the proximal mesh, thereby causing the distal third to move (e.g. bend, curve, deform, fold, and/or invert) in a proximal direction.

In an example, embolic material of this device can be a congealing liquid or gel. In an example, embolic material of this device can be a liquid or gel which congeals in an aneurysm sac. In an example, embolic members of this device can be hydrogel pieces and/or particles. In an example, embolic members of this device can comprise string-of-pearls longitudinal sequences of embolic pieces (e.g. beads, hydrogels, or microsponges) which connected by flexible longitudinal wires, sutures, strings, threads, or yarns. In an example, embolic members of this device can comprise polymer embolic coils. In an example, embolic members of this device can comprise solid or hollow polymer strands. In an example, embolic members of this device can comprise solid or hollow polymer loops.

In an example, this device can comprise a first catheter and/or lumen through which a proximal mesh (e.g. neck bridge) is delivered into an aneurysm sac and a second catheter and/or lumen through which embolic members and/or material is delivered to a space between the proximal mesh and the wall (e.g. distal wall or dome) of the aneurysm sac. In an example, this device can comprise a first catheter and/or lumen through which an proximal mesh is delivered into an aneurysm sac and a second catheter and/or lumen through which embolic members and/or material is delivered to a space between the proximal mesh and the wall (e.g. distal wall or dome) of the aneurysm sac, wherein the second catheter and/or lumen slides through a central opening in the proximal mesh (e.g. neck bridge).

In an example, this device can comprise a first catheter and/or lumen through which an proximal mesh is delivered into an aneurysm sac and a second catheter and/or lumen through which embolic members and/or material is delivered to a space between the proximal mesh and the wall (e.g. distal wall or dome) of the aneurysm sac, wherein the second catheter and/or lumen is detachably-connected to a non-central opening in the proximal mesh (e.g. neck bridge). In an example, this device can comprise a first catheter and/or lumen through which an proximal mesh is delivered into an aneurysm sac and a second catheter and/or lumen through which embolic members and/or material is delivered to a space between the proximal mesh and the wall (e.g. distal wall or dome) of the aneurysm sac, wherein the first and second catheter and/or lumens are inserted simultaneously into a person's vasculature.

In an example, this device can comprise a first catheter and/or lumen through which an proximal mesh is delivered into an aneurysm sac and a second catheter and/or lumen through which embolic members and/or material is delivered to a space between the proximal mesh and the wall (e.g. distal wall or dome) of the aneurysm sac, wherein the first and second catheter and/or lumens are nested, coaxial, and/or concentric relative to each other. In an example, this device can comprise a first catheter and/or lumen through which an proximal mesh is delivered into an aneurysm sac and a second catheter and/or lumen through which embolic members and/or material is delivered to a space between the proximal mesh and the wall (e.g. distal wall or dome) of the aneurysm sac, wherein the second catheter and/or lumen is detachably-connected to the proximal mesh (e.g. neck bridge).

In an example, this device can comprise a first catheter and/or lumen through which an proximal mesh is delivered into an aneurysm sac and a second catheter and/or lumen through which embolic members and/or material is delivered to a space between the proximal mesh and the wall (e.g. distal wall or dome) of the aneurysm sac, wherein the second catheter and/or lumen is detachably-connected to a distal portion of the proximal mesh (e.g. neck bridge). In an example, this device can comprise a first catheter and/or lumen through which a proximal mesh (e.g. neck bridge) is delivered into an aneurysm sac and a second catheter and/or lumen through which embolic members and/or material is delivered to a space between the proximal mesh and the wall (e.g. distal wall or dome) of the aneurysm sac, wherein the first catheter and/or lumen has a 50% larger diameter than the second catheter and/or lumen. In an example, this device can comprise a first catheter through which a proximal mesh (e.g. neck bridge) is delivered into an aneurysm sac and a second catheter through which embolic members and/or material is delivered to a space between the proximal mesh and the wall (e.g. distal wall or dome) of the aneurysm sac.

In an example, a method for occluding an aneurysm can comprise: inserting a proximal mesh (e.g. neck bridge) into an aneurysm sac; expanding the proximal mesh into a first shape, wherein this first shape is convex; inserting a catheter through a central opening in the proximal mesh into a space between the proximal mesh and the wall (e.g. distal wall or dome) of the aneurysm sac; inserting embolic members and/or embolic material into this space, wherein accumulation of the embolic members and/or embolic material compresses, folds, and/or inverts a distal portion of the proximal mesh into a proximal portion of the proximal mesh, wherein this compression, folding, and/or inversion changes the proximal mesh into a second shape, wherein this second shape is a bowl (e.g. hemisphere, semi-ellipsoid, inverted umbrella, or cup) shape; and withdrawing the catheter from the proximal mesh and the aneurysm sac.

In an example, a method for occluding an aneurysm can comprise: inserting a proximal mesh (e.g. neck bridge) into an aneurysm sac; expanding the proximal mesh into a convex (e.g. spherical, ellipsoidal, ovaloid, prolate spheroid, and/or generally globular) shape; and inserting embolic members and/or embolic material through central opening in the proximal mesh into space between the proximal mesh and the wall (e.g. distal wall or dome) of the aneurysm sac, wherein accumulation of the embolic member and/or embolic material in this space compresses the distal surface of the proximal mesh in a proximal direction, thereby deforming, folding, and/or inverting the proximal mesh into a bowl shape (e.g. hemispherical, semi-ellipsoidal, inverted umbrella shape, or cup shape).

In an example, a central inversion of a globular proximal mesh can form a lumen through the proximal mesh, wherein embolic members and/or embolic material is inserted through this lumen into a space between the proximal mesh and the wall (e.g. distal wall or dome) of an aneurysm sac. In an example, a central inversion of the distal half a proximal mesh can form a lumen through the proximal mesh, wherein embolic members and/or embolic material is inserted through this lumen into a space between the proximal mesh and the wall (e.g. distal wall or dome) of an aneurysm sac.

In an example, a device can further comprise a columnar pathway (e.g. a catheter, tube, mesh column, or channel) through the interior of a proximal mesh along a central proximal-to-distal axis of the proximal mesh, wherein embolic members and/or embolic material is inserted through this pathway into a space between the proximal mesh and the wall (e.g. distal wall or dome) of an aneurysm sac. In an example, a device can further comprise a hyperbolic-shaped pathway (e.g. a catheter, tube, or mesh column) through the interior of a proximal mesh along a central proximal-to-distal axis of the proximal mesh, wherein embolic members and/or embolic material is inserted through this pathway into a space between the proximal mesh and the wall (e.g. distal wall or dome) of an aneurysm sac.

In an example, this device can further comprise a closure mechanism which closes an opening and/or pathway through a proximal mesh after embolic members and/or embolic material has been inserted through the opening and/or pathway. In an example, this device can further comprise a closure mechanism which closes an opening and/or pathway through a proximal mesh after embolic members and/or embolic material has been inserted through the opening and/or pathway, wherein this closure mechanism is remotely activated by a person operating the device by the application of electrical energy to the closure.

In an example, this device can further comprise a closure mechanism which closes an opening and/or pathway through a proximal mesh after embolic members and/or embolic material has been inserted through the opening and/or pathway, wherein this closure mechanism is a one-way valve. In an example, this device can further comprise a closure mechanism which closes an opening and/or pathway through a proximal mesh after embolic members and/or embolic material has been inserted through the opening and/or pathway, wherein this closure mechanism is a tapered plug.

In an example, a method for occluding an aneurysm can comprise; inserting a proximal mesh (e.g. neck bridge) into an aneurysm sac; expanding the proximal mesh into a generally-globular shape; weaking a circumferential band around the proximal mesh by the application of electrical energy to the band; inserting embolic members and/or embolic material between the proximal mesh and the wall (e.g. distal wall or dome) of the aneurysm sac; and compressing, folding, and/or inverting the proximal mesh into a multi-layer bowl (e.g. hemisphere, semi-ellipsoid, inverted umbrella, or cup) shape. In an example, a proximal mesh (e.g. neck bridge) can comprise a proximal portion, a distal portion, and a weaker portion between the proximal and distal portions, wherein the proximal portion comprises between 30% and 45% of the proximal mesh, wherein the distal portion comprises between 30% and 45% of the proximal mesh, wherein the weaker portion comprises between 5% and 25% of the proximal mesh, and wherein the weaker portion is more elastic and/or more flexible than the proximal and distal portions, thereby facilitating folding and/or inversion of the distal portion into the proximal portion along this transition portion when embolic members and/or embolic material pushes on the distal portion.

In an example, a proximal mesh (e.g. neck bridge) can have a convex (e.g. globular) shape when it is first expanded within an aneurysm sac, wherein the proximal mesh further comprises a circumferential portion (e.g. equatorial band) which is weakened by the application of electrical energy, wherein this weakening facilitates deformation, inversion, and/or folding of the proximal mesh along this circumferential portion. In an example, the equatorial band can play Samba music. In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck, a distal portion which is farther from the aneurysm neck, and an intermediate portion (e.g. a circumferential or equatorial band) between the proximal and distal portions, wherein the intermediate portion is more flexible, more elastic, less dense, thinner, and/or weaker than the proximal and distal portions, and wherein the intermediate portion comprises less than 10% of the proximal mesh.

In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck, a distal portion which is farther from the aneurysm neck, and an intermediate portion (e.g. a circumferential or equatorial band) between the proximal and distal portions, wherein the intermediate portion is more flexible, more elastic, less dense, thinner, and/or weaker than the proximal and distal portions, and wherein the intermediate portion spans between 10% and 20% of the proximal-to-distal axis of the proximal mesh.

In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck, a distal portion which is farther from the aneurysm neck, and an intermediate portion (e.g. a circumferential or equatorial band) between the proximal and distal portions, wherein the intermediate portion is more flexible, more elastic, less dense, thinner, and/or weaker than the proximal and distal portions, and wherein the intermediate portion can be selectively weakened by the application of electrical energy to facilitate movement (e.g. distortion, folding, and/or inversion) of the distal portion toward the proximal portion.

In an example, a proximal mesh can be made with braided or woven longitudinal members (e.g. filaments, wires, tubes, or strands); wherein the proximal mesh has a first configuration after it has been expanded in an aneurysm sac, but before a distal portion of the proximal mesh has been moved in a proximal direction; wherein the proximal mesh has a second configuration after a distal portion of the proximal mesh has been moved in a proximal direction; wherein first ends of the longitudinal members converge (e.g. meet or are attached together) at a proximal hub in the first configuration; and wherein second ends of the longitudinal members converge (e.g. meet or are attached together) at a distal hub in the first configuration.

In an example, a proximal mesh can be made with braided or woven longitudinal members (e.g. filaments, wires, tubes, or strands); wherein the proximal mesh has a first configuration after it has been expanded in an aneurysm sac, but before a distal portion of the proximal mesh has been moved in a proximal direction; wherein the proximal mesh has a second configuration after a distal portion of the proximal mesh has been moved in a proximal direction; wherein first ends of the longitudinal members converge (e.g. meet or are attached together) at a proximal hub in the first configuration; wherein second ends of the longitudinal members converge (e.g. meet or are attached together) at a distal hub in the first configuration; and wherein the distal hub is connected with the proximal hub in the second configuration.

In an example, a proximal mesh can be made with braided or woven longitudinal members (e.g. filaments, wires, tubes, or strands), wherein the proximal mesh has a first configuration after it has been expanded in an aneurysm sac, but before a distal portion of the proximal mesh has been moved in a proximal direction, wherein the proximal mesh has a second configuration after a distal portion of the proximal mesh has been moved in a proximal direction, wherein both ends of the longitudinal members meet in a proximal portion of the proximal mesh when the proximal mesh is in the second configuration, and wherein a distal portion of the proximal mesh includes folds of the longitudinal members when the proximal mesh is in the second configuration.

In an example, a proximal mesh can be made with braided or woven longitudinal members (e.g. filaments, wires, tubes, or strands); wherein the proximal mesh has a first configuration after it has been expanded in an aneurysm sac, but before a distal portion of the proximal mesh has been moved in a proximal direction; wherein the proximal mesh has a second configuration after a distal portion of the proximal mesh has been moved in a proximal direction; wherein first ends of the longitudinal members converge (e.g. meet and/or are attached together) in a proximal location of the proximal mesh in the first configuration; wherein second ends of the longitudinal members converge (e.g. meet and/or are attached together) in a distal location of the proximal mesh in the first configuration; and wherein first and second ends of the longitudinal members converge (e.g. meet and/or are attached together) in a proximal location of the proximal mesh in the second configuration.

In an example, a proximal mesh can be made with braided or woven longitudinal members (e.g. filaments, wires, tubes, or strands); wherein the proximal mesh has a first configuration after it has been expanded in an aneurysm sac, but before a distal portion of the proximal mesh has been moved in a proximal direction; wherein the proximal mesh has a second configuration after a distal portion of the proximal mesh has been moved in a proximal direction; wherein first ends of the longitudinal members converge (e.g. meet and/or are attached together) in a proximal hub of the proximal mesh in the first configuration; wherein second ends of the longitudinal members converge (e.g. meet and/or are attached together) in a distal hub of the proximal mesh in the first configuration; wherein first and second hubs converge (e.g. meet and/or are attached together) in the second configuration.

In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to invert and/or fold into (a concavity of) a proximal portion of the proximal mesh. In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to invert and/or fold into (a concavity of) a proximal portion of the proximal mesh. In an alternative example, pulling or rotating a flexible longitudinal member (e.g. wire, suture, or string) which is (detachably) connected to a distal portion (e.g. distal hub) of a proximal mesh can change the distal portion of the mesh from being convex to concave.

In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to bend, curve, deform, fold, and/or invert into a proximal portion of the proximal mesh and changing the proximal mesh into a multi-layer bowl (e.g. hemisphere, semi-ellipsoid, inverted umbrella, or cup) shape. In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to deform into (a concavity of) a proximal portion of the proximal mesh and changing the proximal mesh into a funnel shape (e.g. hyperbolic, parabolic, or frustal shape).

In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion of the globular proximal mesh to change into a funnel shape (e.g. hyperbolic, parabolic, or frustal shape) and the proximal portion of the proximal mesh to change into a bowl shape (e.g. hemispherical, semi-ellipsoidal, inverted umbrella, or cup shape).

In an alternative example, a method for occluding an aneurysm can comprise: inserting a proximal mesh (e.g. neck bridge) into an aneurysm sac and expanding the proximal mesh into a generally-globular shape (e.g. sphere, ellipsoid, oval, or prolate sphere shape); pulling in a proximal direction a wire, string, suture, filament, or other flexible longitudinal member which is attached to a distal portion of the proximal mesh, thereby compressing, folding, and/or inverting the distal portion of the proximal mesh into a proximal portion of the proximal mesh and changing the proximal mesh into a multi-layer bowl (e.g. hemisphere, semi-ellipsoid, inverted umbrella, or cup) shape (e.g. hemisphere, semi-ellipsoid, cup, or funnel shape); and then inserting embolic members and/or embolic material into a space between a distal portion of the proximal member and the wall (e.g. distal wall or dome) of the aneurysm sac.

In an alternative example, a method for occluding an aneurysm can comprise: inserting a proximal mesh (e.g. neck bridge) into an aneurysm sac and expanding the proximal mesh into a generally-globular shape (e.g. sphere, ellipsoid, oval, or prolate sphere shape); pulling in a proximal direction a wire, string, suture, filament, or other flexible longitudinal member which is attached to a distal portion of the proximal mesh, thereby compressing, folding, and/or inverting the distal portion of the proximal mesh into a proximal portion of the proximal mesh and changing the proximal mesh into a multi-layer bowl (e.g. hemisphere, semi-ellipsoid, inverted umbrella, or cup) shape (e.g. hemisphere, semi-ellipsoid, cup, or funnel shape); and then inserting embolic members and/or embolic material through the proximal mesh between the proximal mesh and the wall (e.g. distal wall or dome) of the aneurysm sac.

In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to invert into (a concavity of) a proximal portion of the proximal mesh and changing the proximal mesh into a toroidal (e.g. doughnut or bagel) shape. In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to move (e.g. bend, curve, deform, and/or invert) toward a proximal portion of the proximal mesh and changing the proximal mesh into a toroidal (e.g. doughnut or bagel) shape.

In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to move (e.g. bend, curve, deform, and/or invert) in a proximal direction, thereby changing the proximal mesh into a toroidal (e.g. doughnut or bagel) shape.

In an alternative example, a proximal mesh (e.g. neck bridge) can have a first configuration after having been expanded in an aneurysm sac and a subsequent second configuration after a flexible longitudinal member (e.g. wire, suture, or string) which is (detachably) connected to a distal portion (e.g. distal hub) of the proximal mesh has been pulled or rotated, wherein the interior of proximal mesh is smaller in the second configuration than in the first configuration. In an alternative example, a proximal mesh (e.g. neck bridge) can have a first configuration after having been expanded in an aneurysm sac and a subsequent second configuration after a flexible longitudinal member (e.g. wire, suture, or string) which is (detachably) connected to a distal portion (e.g. distal hub) of the proximal mesh has been pulled or rotated, wherein a distal end and/or hub of the proximal mesh connects to a proximal end and/or hub of the proximal mesh in the second configuration.

In an alternative example, a proximal mesh (e.g. neck bridge) can have a first configuration after having been expanded in an aneurysm sac and a subsequent second configuration after a flexible longitudinal member (e.g. wire, suture, or string) which is (detachably) connected to a distal portion (e.g. distal hub) of the proximal mesh has been pulled or rotated, wherein the proximal mesh has a generally-globular (e.g. sphere, ellipsoid, ovaloid, or prolate sphere) shape in the first configuration and a toroidal and/or doughnut shape in the second configuration. In an alternative example, a proximal mesh (e.g. neck bridge) can have a first configuration after having been expanded in an aneurysm sac and a subsequent second configuration after a flexible longitudinal member (e.g. wire, suture, or string) which is (detachably) connected to a distal portion (e.g. distal hub) of the proximal mesh has been pulled or rotated, wherein the proximal mesh has a half-toroidal and/or sliced-doughnut shape in the second configuration.

In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to invert and/or fold into two-layer bowl (e.g. hemisphere, semi-ellipsoid, inverted umbrella, or cup) shape. In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby decreasing the porosity of the proximal portion of the proximal mesh. In an alternative example, a proximal mesh (e.g. neck bridge) which is inserted into an aneurysm sac—after it has been expanded in the aneurysm sac but before it has been compressed by pulling or rotating a flexible longitudinal member (e.g. wire, suture, or string)—can have a proximal portion which is closer to the aneurysm neck and a distal portion which is farther from the aneurysm neck, wherein the distal portion spans between 20% and 45% of the proximal-to-distal axis of the proximal mesh.

In an alternative example, a proximal mesh (e.g. neck bridge) can have a convex (e.g. globular) shape when it is first expanded within an aneurysm sac, wherein the proximal mesh further comprises a weaker circumferential portion (e.g. equatorial band) with greater flexibility, greater elasticity, greater compliance, and/or thinner width than the rest of the proximal mesh, wherein this encourages the distal surface of the proximal mesh to deform, invert, and/or fold along this circumferential portion when a flexible longitudinal member (e.g. wire, suture, or string) attached to the proximal mesh is pulled or rotated.

In an alternative example, a proximal mesh (e.g. neck bridge) can comprise a proximal portion, a distal portion, and a weaker portion between the proximal and distal portions, wherein the proximal portion comprises between 30% and 45% of the proximal mesh, wherein the distal portion comprises between 30% and 45% of the proximal mesh, wherein the weaker portion is less than 10% of the proximal mesh, and wherein the weaker portion is more elastic and/or more flexible than the proximal and distal portions, thereby facilitating folding and/or inversion of the distal portion into the proximal portion along this transition portion when a flexible longitudinal member (e.g. wire, suture, or string) attached to the proximal mesh is pulled or rotated.

In an alternative example, a proximal mesh (e.g. neck bridge) which is inserted into an aneurysm sac—after it has been expanded in the aneurysm sac but before it has been compressed by pulling or rotating a flexible longitudinal member (e.g. wire, suture, or string)—can have a proximal portion which is closer to the aneurysm neck, a distal portion which is farther from the aneurysm neck, and an intermediate portion (e.g. a circumferential or equatorial band) between the proximal and distal portions, wherein the intermediate portion is more flexible, more elastic, less dense, thinner, and/or weaker than the proximal and distal portions, and wherein the intermediate portion spans between 5% and 15% of the proximal-to-distal axis of the proximal mesh.

In an alternative example, a proximal mesh (e.g. neck bridge) which is inserted into an aneurysm sac—after it has been expanded in the aneurysm sac but before it has been compressed by pulling or rotating a flexible longitudinal member (e.g. wire, suture, or string)—can have a proximal portion which is closer to the aneurysm neck, a distal portion which is farther from the aneurysm neck, and an intermediate portion (e.g. a circumferential or equatorial band) between the proximal and distal portions, wherein the intermediate portion is more flexible, more elastic, less dense, thinner, and/or weaker than the proximal and distal portions, and wherein the intermediate portion can be selectively weakened by the application of electrical energy.

In an example, accumulation of embolic members and/or material in a space between a proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) in a proximal direction. In an example, insertion of embolic members and/or material into a space between a proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) toward a proximal portion of the proximal mesh.

In an example, accumulation of embolic members and/or material in a space between a proximal mesh (e.g. neck bridge) in an aneurysm sac and a wall (e.g. the distal wall or dome) of the aneurysm sac can push, press, and/or deform a distal portion of the proximal mesh and decreasing the concavity of the distal portion of the mesh (relative to a proximal portion of the proximal mesh). In an example, accumulation of embolic members and/or material in a space between a proximal mesh (e.g. neck bridge) in an aneurysm sac and a wall (e.g. the distal wall or dome) of the aneurysm sac can push, press, and/or deform a distal portion of the proximal mesh and decreasing the convexity of the distal portion of the mesh (relative to a proximal portion of the proximal mesh). In an example, accumulation of embolic members and/or material in a space between a proximal mesh (e.g. neck bridge) in an aneurysm sac and a wall (e.g. the distal wall or dome) of the aneurysm sac can push, press, and/or deform a distal portion of the proximal mesh and changing the convexity of the distal portion of the mesh from facing in a distal direction to facing in a proximal direction.

In an example, accumulation of embolic members and/or material in a space between a proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) into a proximal portion of the proximal mesh. In an example, accumulation of embolic members and/or material in a space between a proximal mesh (e.g. neck bridge) in an aneurysm sac and a wall (e.g. the distal wall or dome) of the aneurysm sac can push, press, and/or deform a distal portion of the proximal mesh, thereby inverting the distal portion of the proximal mesh into the proximal portion of the proximal mesh. In an example, insertion of embolic members and/or material into a space between a proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) into a proximal portion of the proximal mesh.

In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein a distal end and/or hub of the proximal mesh does not contact the proximal end and/or hub of the proximal mesh in the first configuration, but does contact the proximal end/or hub in the second configuration.

In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein the distal surface of the proximal mesh curves outward in a distal direction in the first configuration and has a funnel shape (curving in a proximal direction) in the second configuration.

In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein the distal surface of the proximal mesh is distally-convex in the first configuration and has a bowl (e.g. hemisphere, semi-ellipsoid, inverted umbrella, or cup) shape (curving in a proximal direction) in the second configuration. In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein the proximal mesh has a toroidal and/or doughnut shape in the second configuration.

In an example, a proximal mesh (e.g. neck bridge) can have a first configuration after having been expanded in an aneurysm sac and a subsequent second configuration after having been compressed in a distal-to-proximal direction by accumulation of embolic members and/or embolic material which has been inserted into a space between the proximal mesh and the wall (e.g. the distal wall or dome) of the aneurysm sac, wherein the proximal mesh has a bowl (e.g. hemisphere, semi-ellipsoid, inverted umbrella, or cup) shape in the second configuration. In an example, a proximal mesh (e.g. neck bridge) can have: a first configuration with a distal end and/or hub and a proximal end and/or hub after having been expanded in an aneurysm sac; and a second configuration after having been compressed in a distal-to-proximal direction by accumulation of embolic members and/or embolic material which has been inserted into a space between the proximal mesh and the wall (e.g. the distal wall or dome) of the aneurysm sac; wherein the distal end and/or hub contacts the proximal end and/or hub in the second configuration.

In an example, insertion of embolic members and/or material into a space between a two-layer globular (e.g. spherical, ellipsoidal, ovaloid, prolate spherical, apple, or barrel shaped) proximal mesh (e.g. neck bridge) in an aneurysm sac and a wall (e.g. the distal wall or dome) of the aneurysm sac can push, press, and/or deform a distal portion of the proximal mesh in a proximal direction, thereby causing the distal portion to invert and/or fold into four-layer bowl (e.g. hemisphere, semi-ellipsoid, inverted umbrella, or cup) shape. In an example, insertion of embolic members and/or material into a space between a mesh (e.g. neck bridge) with a first shape and a first level of wall density in an aneurysm sac and a wall (e.g. the distal wall or dome) of the aneurysm sac can push, press, and/or deform a distal portion of the mesh in a proximal direction, thereby causing the mesh to compress, fold, and/or invert into a second shape and a second level of wall density, wherein the second level of wall density is greater than the first level of wall density.

In an example, insertion of embolic members and/or material into a space between a mesh (e.g. neck bridge) with a first shape and a first level of wall porosity in an aneurysm sac and a wall (e.g. the distal wall or dome) of the aneurysm sac can push, press, and/or deform a distal portion of the mesh in a proximal direction, thereby causing the mesh to compress, fold, and/or invert into a second shape and a second level of wall porosity, wherein the second level of wall porosity is less than the first level of wall porosity.

In an example, accumulation of embolic members and/or material in a space between a globular (e.g. spherical, ellipsoidal, ovaloid, prolate spherical, apple, or barrel shaped) proximal mesh (e.g. neck bridge) in an aneurysm sac and a wall (e.g. the distal wall or dome) of the aneurysm sac can push, press, and/or deform a distal portion of the proximal mesh, thereby causing the distal portion to invert and/or fold into (a concavity of) a proximal portion of the proximal mesh and changing the proximal mesh into a multi-layer bowl (e.g. hemisphere, semi-ellipsoid, inverted umbrella, or cup) shape.

In an example, accumulation of embolic members and/or material in a space between a globular (e.g. spherical, ellipsoidal, ovaloid, prolate spherical, apple, or barrel shaped) proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) toward a proximal portion of the proximal mesh and changing the proximal mesh into a multi-layer bowl (e.g. hemisphere, semi-ellipsoid, inverted umbrella, or cup) shape. In an example, insertion of embolic members and/or material into a space between a globular (e.g. spherical, ellipsoidal, ovaloid, prolate spherical, apple, or barrel shaped) proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) in a proximal direction and changing the proximal mesh into a multi-layer bowl (e.g. hemisphere, semi-ellipsoid, inverted umbrella, or cup) shape.

In an example, accumulation of embolic members and/or material in a space between a convex proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) into a proximal portion of the proximal mesh and changing the proximal mesh into a funnel shape (e.g. hyperbolic, parabolic, or frustal shape). In an example, insertion of embolic members and/or material into a space between a convex proximal mesh (e.g. neck bridge) in an aneurysm sac and a wall (e.g. the distal wall or dome) of the aneurysm sac can push, press, and/or deform a distal portion of the proximal mesh, thereby causing the distal portion to deform into (a concavity of) a proximal portion of the proximal mesh and changing the proximal mesh into a funnel shape (e.g. hyperbolic, parabolic, or frustal shape).

In an example, insertion of embolic members and/or material into a space between a convex proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, fold, and/or invert) toward a proximal portion of the proximal mesh and changing the proximal mesh into a funnel shape (e.g. hyperbolic, parabolic, or frustal shape). In an example, accumulation of embolic members and/or material in a space between a convex proximal mesh (e.g. neck bridge) in an aneurysm sac and a wall (e.g. the distal wall or dome) of the aneurysm sac can push, press, and/or deform a distal portion of the proximal mesh, thereby causing the distal portion to invert into (a concavity of) a proximal portion of the proximal mesh and changing the proximal mesh into a toroidal (e.g. doughnut or bagel) shape.

In an example, accumulation of embolic members and/or material in a space between a convex proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, and/or invert) toward a proximal portion of the proximal mesh and changing the proximal mesh into a toroidal (e.g. doughnut or bagel) shape. In an example, insertion of embolic members and/or material into a space between a convex proximal mesh (e.g. neck bridge) in an aneurysm sac and a wall (e.g. the distal wall or dome) of the aneurysm sac can push, press, and/or deform a distal portion of the proximal mesh, thereby causing the distal portion to move (e.g. bend, curve, deform, and/or invert) in a proximal direction, thereby changing the proximal mesh into a toroidal (e.g. doughnut or bagel) shape. In an example, the interior of a proximal mesh can have a barrel shape. In an example, the interior of a proximal mesh can have a frustal shape. In an example, the interior of a proximal mesh can have a half globular shape. In an example, the interior of a proximal mesh can have a half-toroidal (e.g. sliced bagel) shape. In an example, the interior of a proximal mesh can have a toroidal shape.

In an example, a distal portion of a proximal mesh can comprise an opening and/or aperture through which embolic members and/or embolic material pass into a space between the proximal mesh and the aneurysm wall. In an example, a distal portion of a proximal mesh can comprise a funnel-shaped opening and/or aperture through which embolic members and/or embolic material pass into a space between the proximal mesh and the aneurysm wall. In an example, a distal portion of a proximal mesh can comprise an opening and/or aperture through which embolic members and/or embolic material pass into a space between the proximal mesh and the aneurysm wall, wherein this opening and/or aperture can be closed after embolic members and/or embolic material has been inserted into this space.

In an example, a distal portion of a proximal mesh can comprise an opening and/or aperture through which embolic members and/or embolic material pass into a space between the proximal mesh and the aneurysm wall, wherein this opening and/or aperture can be closed by activation of a valve after embolic members and/or embolic material has been inserted into this space. In an example, a proximal mesh can have a distal hub, wherein distal ends of filaments comprising the mesh are connected (e.g. pinched, compressed, glued, and/or welded) in the hub.

In an example, a proximal mesh can have a distal hub; wherein there is an opening and/or lumen through this hub through which embolic members and/or embolic material is inserted into a space between the hub and the aneurysm wall; wherein this hub comprises two (inner and outer) rings, bands, or cylinders; wherein the rings, bands, or cylinders are nested and/or concentric;

wherein embolic members and/or embolic material is inserted into the space through the inner ring, band, or cylinder; and wherein filaments comprising the proximal mesh pass between the inner and outer rings, bands, or cylinders. In an example, distal ends of filaments comprising a proximal mesh can be connected together at a distal hub, wherein this distal hub is moved in a proximal direction by insertion of embolic members and/or embolic material through this hub into a space between the hub and the wall (e.g. distal wall or dome) of an aneurysm sac.

In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck and a distal portion which is farther from the aneurysm neck, wherein the proximal portion spans between 20% and 45% of the proximal-to-distal axis of the proximal mesh. In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck and a distal portion which is farther from the aneurysm neck, wherein the proximal portion spans between 45% and 545% of the proximal-to-distal axis of the proximal mesh.

In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck and a distal portion which is farther from the aneurysm neck, wherein the distal portion spans between 20% and 45% of the proximal-to-distal axis of the proximal mesh. In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck and a distal portion which is farther from the aneurysm neck, wherein the distal portion spans between 45% and 545% of the proximal-to-distal axis of the proximal mesh.

In an example, accumulation of embolic members and/or material in a space between a proximal mesh (e.g. neck bridge) in an aneurysm sac and a wall (e.g. the distal wall or dome) of the aneurysm sac can push and/or force a distal half of the proximal mesh and changing the concavity of the distal half of the mesh (relative to a proximal half of the proximal mesh). In an example, accumulation of embolic members and/or material in a space between a proximal mesh (e.g. neck bridge) in an aneurysm sac and a wall (e.g. the distal wall or dome) of the aneurysm sac can push and/or force a distal half of the proximal mesh and changing the convexity of the distal half of the mesh (relative to a proximal half of the proximal mesh).

In an example, accumulation of embolic members and/or material in a space between a proximal mesh (e.g. neck bridge) in an aneurysm sac and a wall (e.g. the distal wall or dome) of the aneurysm sac can push and/or force a distal half of the proximal mesh and changing the distal half of the mesh from being convex to concave (relative to a proximal half of the proximal mesh). In an example, accumulation of embolic members and/or material in a space between a proximal mesh (e.g. neck bridge) in an aneurysm sac and a wall (e.g. the distal wall or dome) of the aneurysm sac can push and/or force a distal half of the proximal mesh, thereby causing the distal half to move (e.g. bend, curve, deform, fold, and/or invert) toward a proximal half of the proximal mesh.

In an example, insertion of embolic members and/or material into a space between a proximal mesh (e.g. neck bridge) in an aneurysm sac and a wall (e.g. the distal wall or dome) of the aneurysm sac can push and/or force a distal half of the proximal mesh, thereby causing the distal half to move (e.g. bend, curve, deform, fold, and/or invert) in a proximal direction. In an example, insertion of embolic members and/or material into a space between a proximal mesh (e.g. neck bridge) in an aneurysm sac and a wall (e.g. the distal wall or dome) of the aneurysm sac can push and/or force a distal half of the proximal mesh, thereby causing the distal half to move (e.g. bend, curve, deform, fold, and/or invert) into a proximal half of the proximal mesh.

In an example, accumulation of embolic members and/or material in a space between a proximal mesh (e.g. neck bridge) in an aneurysm sac and a wall (e.g. the distal wall or dome) of the aneurysm sac can push and/or force a distal third of the proximal mesh, thereby causing the distal third to move (e.g. bend, curve, deform, fold, and/or invert) into a proximal third of the proximal mesh. In an example, insertion of embolic members and/or material into a space between a proximal mesh (e.g. neck bridge) in an aneurysm sac and a wall (e.g. the distal wall or dome) of the aneurysm sac can push and/or force a distal third of the proximal mesh, thereby causing the distal third to invert and/or fold into (a concavity of) a proximal third of the proximal mesh. In an example, insertion of embolic members and/or material into a space between a proximal mesh (e.g. neck bridge) in an aneurysm sac and a wall (e.g. the distal wall or dome) of the aneurysm sac can push and/or force a distal third of the proximal mesh, thereby causing the distal third to move (e.g. bend, curve, deform, fold, and/or invert) toward a proximal third of the proximal mesh.

In an example, embolic material of this device can be a crosslinking material. In an example, embolic material of this device can be a polymer liquid or gel which congeals in an aneurysm sac. In an example, embolic members of this device can be microbeads and/or microspheres. In an example, embolic members of this device can comprise embolic pieces which are connected by wires, sutures, strings, threads, or yarns. In an example, embolic members of this device can comprise polymer strands, tubes, noodles, or loops. In an example, embolic members of this device can comprise solid or hollow polymer tubes.

In an example, a method for occluding an aneurysm can comprise: inserting a proximal mesh (e.g. neck bridge) into an aneurysm sac; expanding the proximal mesh into a convex (e.g. spherical, ellipsoidal, ovaloid, prolate spheroid, and/or generally globular) shape; inserting a catheter through the proximal mesh into a space between the proximal mesh and the wall (e.g. distal wall or dome) of the aneurysm; and inserting embolic members and/or embolic material through the catheter into this space, wherein accumulation of the embolic member and/or embolic material in this space compresses the distal surface of the proximal mesh in a proximal direction, thereby deforming, folding, and/or inverting the proximal mesh into a bowl shape (e.g. hemispherical, semi-ellipsoidal, inverted umbrella shape, or cup shape).

In an example, this device can comprise a first catheter and/or lumen through which an proximal mesh is delivered into an aneurysm sac and a second catheter and/or lumen through which embolic members and/or material is delivered to a space between the proximal mesh and the wall (e.g. distal wall or dome) of the aneurysm sac, wherein the second catheter and/or lumen passes through a central opening in the proximal mesh (e.g. neck bridge). In an example, this device can comprise a first catheter and/or lumen through which an proximal mesh is delivered into an aneurysm sac and a second catheter and/or lumen through which embolic members and/or material is delivered to a space between the proximal mesh and the wall (e.g. distal wall or dome) of the aneurysm sac, wherein the second catheter and/or lumen slides through a non-central opening in the proximal mesh (e.g. neck bridge).

In an example, this device can comprise a first catheter and/or lumen through which an proximal mesh is delivered into an aneurysm sac and a second catheter and/or lumen through which embolic members and/or material is delivered to a space between the proximal mesh and the wall (e.g. distal wall or dome) of the aneurysm sac, wherein the first and second catheter and/or lumens are inserted simultaneously into the aneurysm sac. In an example, this device can comprise a first catheter and/or lumen through which an proximal mesh is delivered into an aneurysm sac and a second catheter and/or lumen through which embolic members and/or material is delivered to a space between the proximal mesh and the wall (e.g. distal wall or dome) of the aneurysm sac, wherein the first and second catheter and/or lumens are inserted sequentially into a person's vasculature.

In an example, this device can comprise a first catheter and/or lumen through which an proximal mesh is delivered into an aneurysm sac and a second catheter and/or lumen through which embolic members and/or material is delivered to a space between the proximal mesh and the wall (e.g. distal wall or dome) of the aneurysm sac, wherein the first catheter and/or lumen passes through a central opening in the proximal mesh (e.g. neck bridge). In an example, this device can comprise a first catheter and/or lumen through which an proximal mesh is delivered into an aneurysm sac and a second catheter and/or lumen through which embolic members and/or material is delivered to a space between the proximal mesh and the wall (e.g. distal wall or dome) of the aneurysm sac, wherein the second catheter and/or lumen slides through a hub and/or opening in the proximal mesh (e.g. neck bridge).

In an example, this device can comprise a first catheter and/or lumen through which a proximal mesh (e.g. neck bridge) is delivered into an aneurysm sac and a second catheter and/or lumen through which embolic members and/or material is delivered to a space between the proximal mesh and the wall (e.g. distal wall or dome) of the aneurysm sac, wherein the first catheter and/or lumen has a larger diameter than the second catheter and/or lumen. In an example, this device can comprise a first catheter and/or lumen through which a proximal mesh (e.g. neck bridge) is delivered into an aneurysm sac and a second catheter and/or lumen through which embolic members and/or material is delivered to a space between the proximal mesh and the wall (e.g. distal wall or dome) of the aneurysm sac, wherein the second catheter and/or lumen has a 50% larger diameter than the first catheter and/or lumen.

In an example, this device can comprise a first lumen through which an proximal mesh (e.g. neck bridge) is delivered into an aneurysm sac and a second lumen through which embolic members and/or material is delivered to a space between the proximal mesh and the wall (e.g. distal wall or dome) of the aneurysm sac. In an example, a method for occluding an aneurysm can comprise: inserting a proximal mesh (e.g. neck bridge) into an aneurysm sac; expanding the proximal mesh into a first shape, wherein this first shape is convex; inserting a catheter through a central opening in the proximal mesh into a space between the proximal mesh and the wall (e.g. distal wall or dome) of the aneurysm sac; inserting embolic members and/or embolic material into this space, wherein accumulation of the embolic members and/or embolic material compresses, folds, and/or inverts a distal portion of the proximal mesh into a proximal portion of the proximal mesh, wherein this compression, folding, and/or inversion changes the proximal mesh into a second shape, wherein this second shape is a bowl (e.g. hemisphere, semi-ellipsoid, inverted umbrella, or cup) shape; withdrawing the catheter from the proximal mesh and the aneurysm sac; and closing the opening in the proximal mesh.

In an example, a method for occluding an aneurysm can comprise: inserting a proximal mesh (e.g. neck bridge) into an aneurysm sac; expanding the proximal mesh into a convex (e.g. spherical, ellipsoidal, ovaloid, prolate spheroid, and/or generally globular) shape; and inserting embolic members and/or embolic material along the central proximal-to-distal axis of the proximal mesh into space between the proximal mesh and the wall (e.g. distal wall or dome) of the aneurysm sac, wherein accumulation of the embolic member and/or embolic material in this space compresses the distal surface of the proximal mesh in a proximal direction, thereby deforming, folding, and/or inverting the proximal mesh into a bowl shape (e.g. hemispherical, semi-ellipsoidal, inverted umbrella shape, or cup shape).

In an example, a central inversion of a proximal mesh can form a lumen through the proximal mesh, wherein embolic members and/or embolic material is inserted through this lumen into a space between the proximal mesh and the wall (e.g. distal wall or dome) of an aneurysm sac.

In an example, a central inversion of the distal half of a convex proximal mesh can form a lumen through the proximal mesh, wherein embolic members and/or embolic material is inserted through this lumen into a space between the proximal mesh and the wall (e.g. distal wall or dome) of an aneurysm sac.

In an example, a device can further comprise a frustum-shaped pathway (e.g. a catheter, tube, or mesh column) through the interior of a proximal mesh along a central proximal-to-distal axis of the proximal mesh, wherein embolic members and/or embolic material is inserted through this pathway into a space between the proximal mesh and the wall (e.g. distal wall or dome) of an aneurysm sac. In an example, a device can further comprise an axially-central opening (e.g. hole, ring, or valve) through a proximal mesh, wherein embolic members and/or embolic material is inserted through this opening into a space between the proximal mesh and the wall (e.g. distal wall or dome) of an aneurysm sac.

In an example, this device can further comprise a closure mechanism which closes an opening and/or pathway through a proximal mesh after embolic members and/or embolic material has been inserted through the opening and/or pathway, wherein this closure mechanism is remotely activated by a person operating the device. In an example, this device can further comprise a closure mechanism which closes an opening and/or pathway through a proximal mesh after embolic members and/or embolic material has been inserted through the opening and/or pathway, wherein this closure mechanism is remotely activated by the application of electrical energy. In an example, this device can further comprise a closure mechanism which closes an opening and/or pathway through a proximal mesh after embolic members and/or embolic material has been inserted through the opening and/or pathway, wherein this closure mechanism is a leaflet valve. In an example, this device can further comprise a closure mechanism which closes an opening and/or pathway through a proximal mesh after embolic members and/or embolic material has been inserted through the opening and/or pathway, wherein this closure mechanism is a rotatable valve.

In an example, a method for occluding an aneurysm can comprise; inserting a proximal mesh (e.g. neck bridge) into an aneurysm sac; expanding the proximal mesh into a globular shape; weaking a circumferential band around the proximal mesh by the application of electrical energy to the band; inserting embolic members and/or embolic material between the proximal mesh and the wall of the aneurysm sac; and compressing, folding, and/or inverting the proximal mesh into a multi-layer bowl (e.g. hemisphere, semi-ellipsoid, inverted umbrella, or cup) shape which is pressed against the inside of the neck of the aneurysm sac.

In an example, a proximal mesh (e.g. neck bridge) can comprise a proximal portion, a distal portion, and a weaker portion between the proximal and distal portions, wherein the proximal portion comprises between 30% and 45% of the proximal mesh, wherein the distal portion comprises between 30% and 45% of the proximal mesh, wherein the weaker portion is less than 10% of the proximal mesh, and wherein the weaker portion is more elastic and/or more flexible than the proximal and distal portions, thereby facilitating folding and/or inversion of the distal portion into the proximal portion along this transition portion when embolic members and/or embolic material pushes on the distal portion. In an example, a proximal mesh (e.g. neck bridge) can have a convex (e.g. globular) shape when it is first expanded within an aneurysm sac, wherein the proximal mesh further comprises a circumferential portion (e.g. equatorial band) which is weakened by the application of electrical energy after expansion of the proximal mesh, wherein this weakening facilitates deformation, inversion, and/or folding of the proximal mesh along this circumferential portion.

In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck, a distal portion which is farther from the aneurysm neck, and an intermediate portion (e.g. a circumferential or equatorial band) between the proximal and distal portions, wherein the intermediate portion is more flexible, more elastic, less dense, thinner, and/or weaker than the proximal and distal portions, and wherein the intermediate portion spans less than 10% of the proximal-to-distal axis of the proximal mesh.

In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck, a distal portion which is farther from the aneurysm neck, and an intermediate portion (e.g. a circumferential or equatorial band) between the proximal and distal portions, wherein the intermediate portion is more flexible, more elastic, less dense, thinner, and/or weaker than the proximal and distal portions, and wherein the intermediate portion spans between 15% and 35% of the proximal-to-distal axis of the proximal mesh.

In an example, a proximal mesh (e.g. neck bridge) which is inserted into an aneurysm sac (after it has expanded in the aneurysm sac but before it has been compressed by accumulation of embolic members and/or embolic material) can have a proximal portion which is closer to the aneurysm neck, a distal portion which is farther from the aneurysm neck, and an intermediate portion (e.g. a circumferential or equatorial band) between the proximal and distal portions, wherein the intermediate portion is more flexible, more elastic, less dense, thinner, and/or weaker than the proximal and distal portions, and wherein the intermediate portion can be selectively and remotely weakened by the operator of the device by the application of electrical energy to facilitate movement (e.g. distortion, folding, and/or inversion) of the distal portion toward the proximal portion.

In an example, a proximal mesh can be made with braided or woven longitudinal members (e.g. filaments, wires, tubes, or strands); wherein the proximal mesh has a first configuration after it has been expanded in an aneurysm sac, but before a distal portion of the proximal mesh has been moved in a proximal direction; wherein the proximal mesh has a second configuration after a distal portion of the proximal mesh has been moved in a proximal direction; wherein first ends of the longitudinal members converge (e.g. meet or are attached together) at a proximal hub in the first configuration; wherein second ends of the longitudinal members converge (e.g. meet or are attached together) at a distal hub in the first configuration; and wherein the distal hub is moved toward the proximal hub in the second configuration.

In an example, a proximal mesh can be made with braided or woven longitudinal members (e.g. filaments, wires, tubes, or strands), wherein the proximal mesh has a first configuration after it has been expanded in an aneurysm sac, but before a distal portion of the proximal mesh has been moved in a proximal direction, wherein the proximal mesh has a second configuration after a distal portion of the proximal mesh has been moved in a proximal direction, and wherein proximal ends of the longitudinal members meet at a proximal hub and distal ends of the longitudinal members meet at a distal hub when the proximal mesh is in the first configuration.

In an example, a proximal mesh can be made with braided or woven longitudinal members (e.g. filaments, wires, tubes, or strands); wherein the proximal mesh has a first configuration after it has been expanded in an aneurysm sac, but before a distal portion of the proximal mesh has been moved in a proximal direction; wherein the proximal mesh has a second configuration after a distal portion of the proximal mesh has been moved in a proximal direction; wherein first ends of the longitudinal members converge (e.g. meet and/or are attached together) in a proximal location of the proximal mesh in the first configuration; wherein second ends of the longitudinal members converge (e.g. meet and/or are attached together) in a distal location of the proximal mesh in the first configuration; wherein first and second ends of the longitudinal members converge (e.g. meet and/or are attached together) in a proximal location of the proximal mesh in the second configuration; and wherein the distal surface of the proximal mesh includes folds in the longitudinal members in the second configuration.

In an example, a proximal mesh can be made with braided or woven longitudinal members (e.g. filaments, wires, tubes, or strands); wherein the proximal mesh has a first configuration after it has been expanded in an aneurysm sac, but before a distal portion of the proximal mesh has been moved in a proximal direction; wherein the proximal mesh has a second configuration after a distal portion of the proximal mesh has been moved in a proximal direction; wherein first ends of the longitudinal members converge (e.g. meet and/or are attached together) in the proximal half of the proximal mesh in the first configuration; wherein second ends of the longitudinal members converge (e.g. meet and/or are attached together) in the distal half of the proximal mesh in the first configuration; and wherein first and second ends of the longitudinal members converge (e.g. meet and/or are attached together) in the proximal half of the proximal mesh in the second configuration.

In an example, a proximal mesh (e.g. neck bridge) can be centered over an aneurysm neck after it has been expanded within an aneurysm sac and before it has been proximally compressed. In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to move (e.g. bend, curve, deform, fold, and/or invert) into a proximal portion of the proximal mesh. In an alternative example, pulling or rotating a flexible longitudinal member (e.g. wire, suture, or string) which is (detachably) connected to a distal portion (e.g. distal hub) of a proximal mesh can change the concavity of the distal portion of the mesh (relative to a proximal portion of the proximal mesh).

In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby deforming, folding, and/or inverting the proximal mesh into a bowl shape (e.g. hemispherical, semi-ellipsoidal, inverted umbrella shape, or cup shape). In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to move (e.g. bend, curve, deform, fold, and/or invert) toward a proximal portion of the proximal mesh and changing the proximal mesh into a multi-layer bowl (e.g. hemisphere, semi-ellipsoid, inverted umbrella, or cup) shape.

In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to move (e.g. bend, curve, deform, fold, and/or invert) into a proximal portion of the proximal mesh and changing the proximal mesh into a funnel shape (e.g. hyperbolic, parabolic, or frustal shape). In an alternative example, a method for occluding an aneurysm can comprise: inserting a proximal mesh (e.g. neck bridge) into an aneurysm sac and expanding the proximal mesh into a convex shape; pulling in a proximal direction a wire, string, suture, filament, or other flexible longitudinal member which is attached to the distal surface of the proximal mesh, thereby compressing, folding, and/or inverting the distal surface of the proximal mesh into the proximal surface of the proximal mesh and changing the proximal mesh into a bowl (e.g. hemisphere, semi-ellipsoid, inverted umbrella, or cup) shape; and inserting embolic members and/or embolic material into a space between a distal portion of the proximal member and the wall (e.g. distal wall or dome) of the aneurysm sac.

In an alternative example, a method for occluding an aneurysm can comprise: inserting a proximal mesh (e.g. neck bridge) into an aneurysm sac and expanding the proximal mesh into a convex shape; pulling in a proximal direction a wire, string, suture, filament, or other flexible longitudinal member which is attached to the distal surface of the proximal mesh, thereby compressing, folding, and/or inverting the distal surface of the proximal mesh into the proximal surface of the proximal mesh and changing the proximal mesh into a bowl (e.g. hemisphere, semi-ellipsoid, inverted umbrella, or cup) shape; and inserting embolic members and/or embolic material through the proximal mesh between the proximal mesh and the wall (e.g. distal wall or dome) of the aneurysm sac.

In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion of the globular proximal mesh to form a funnel shape (e.g. hyperbolic, parabolic, or frustal shape) and the proximal portion of the proximal mesh to form a bowl shape (e.g. hemispherical, semi-ellipsoidal, inverted umbrella, or cup shape).

In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to move (e.g. bend, curve, deform, and/or invert) into a proximal portion of the proximal mesh and changing the proximal mesh into a toroidal (e.g. doughnut or bagel) shape. In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to invert into (a concavity of) a proximal portion of the proximal mesh and changing the proximal mesh into a toroidal (e.g. doughnut or bagel) shape.

In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to move (e.g. bend, curve, deform, fold, and/or invert) toward a proximal portion of the proximal mesh and changing the proximal mesh into a toroidal (e.g. doughnut or bagel) shape.

In an alternative example, a proximal mesh (e.g. neck bridge) can have a first configuration after having been expanded in an aneurysm sac and a subsequent second configuration after a flexible longitudinal member (e.g. wire, suture, or string) which is (detachably) connected to a distal portion (e.g. distal hub) of the proximal mesh has been pulled or rotated, wherein a distal end and/or hub of the proximal mesh is closer to a proximal end and/or hub of the proximal mesh in the second configuration than in the first configuration. In an alternative example, a proximal mesh (e.g. neck bridge) can have a first configuration after having been expanded in an aneurysm sac and a subsequent second configuration after a flexible longitudinal member (e.g. wire, suture, or string) which is (detachably) connected to a distal portion (e.g. distal hub) of the proximal mesh has been pulled or rotated, wherein a distal surface of the proximal mesh is convex in the first configuration and has a funnel shape in the second configuration.

In an alternative example, a proximal mesh (e.g. neck bridge) can have a first configuration after having been expanded in an aneurysm sac and a subsequent second configuration after a flexible longitudinal member (e.g. wire, suture, or string) which is (detachably) connected to a distal portion (e.g. distal hub) of the proximal mesh has been pulled or rotated, wherein the proximal mesh has a generally-globular (e.g. sphere, ellipsoid, ovaloid, or prolate sphere) shape in the first configuration and a half-toroidal and/or sliced-doughnut shape in the second configuration. In an alternative example, a proximal mesh (e.g. neck bridge) can have a first configuration after having been expanded in an aneurysm sac and a subsequent second configuration after a flexible longitudinal member (e.g. wire, suture, or string) which is (detachably) connected to a distal portion (e.g. distal hub) of the proximal mesh has been pulled or rotated, wherein the proximal mesh has a bowl (e.g. hemisphere, semi-ellipsoid, inverted umbrella, or cup) shape in the second configuration.

In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby increasing the density of the proximal portion of the proximal mesh. In an alternative example, a device can include a flexible longitudinal member (e.g. wire, suture, or string) which is (detachably) connected to a distal portion (e.g. distal hub) of a proximal mesh, wherein pulling or rotating this longitudinal member moves the distal portion of the proximal mesh in a proximal direction, thereby causing the distal portion to invert and/or fold into multi-layer bowl (e.g. hemisphere, semi-ellipsoid, inverted umbrella, or cup) shape. In an alternative example, a proximal mesh (e.g. neck bridge) which is inserted into an aneurysm sac—after it has been expanded in the aneurysm sac but before it has been compressed by pulling or rotating a flexible longitudinal member (e.g. wire, suture, or string)—can have a proximal portion which is closer to the aneurysm neck and a distal portion which is farther from the aneurysm neck, wherein the distal portion comprises between 45% and 55% of the proximal mesh.

In an alternative example, a proximal mesh (e.g. neck bridge) can comprise a proximal portion, a distal portion, and a weaker portion between the proximal and distal portions, wherein the weaker portion is more elastic and/or more flexible than the proximal and distal portions, thereby facilitating folding and/or inversion of the distal portion into the proximal portion along this transition portion when a flexible longitudinal member (e.g. wire, suture, or string) attached to the proximal mesh is pulled or rotated. In an alternative example, a proximal mesh (e.g. neck bridge) which is inserted into an aneurysm sac—after it has been expanded in the aneurysm sac but before it has been compressed by pulling or rotating a flexible longitudinal member (e.g. wire, suture, or string)—can have a proximal portion which is closer to the aneurysm neck, a distal portion which is farther from the aneurysm neck, and an intermediate portion (e.g. a circumferential or equatorial band) between the proximal and distal portions, wherein the intermediate portion is more flexible, more elastic, less dense, thinner, and/or weaker than the proximal and distal portions, and wherein the intermediate portion comprises less than 10% of the proximal mesh.

In an alternative example, a proximal mesh (e.g. neck bridge) which is inserted into an aneurysm sac—after it has been expanded in the aneurysm sac but before it has been compressed by pulling or rotating a flexible longitudinal member (e.g. wire, suture, or string)—can have a proximal portion which is closer to the aneurysm neck, a distal portion which is farther from the aneurysm neck, and an intermediate portion (e.g. a circumferential or equatorial band) between the proximal and distal portions, wherein the intermediate portion is more flexible, more elastic, less dense, thinner, and/or weaker than the proximal and distal portions, and wherein the intermediate portion spans between 10% and 20% of the proximal-to-distal axis of the proximal mesh.

In an alternative example, a proximal mesh (e.g. neck bridge) which is inserted into an aneurysm sac—after it has been expanded in the aneurysm sac but before it has been compressed by pulling or rotating a flexible longitudinal member (e.g. wire, suture, or string)—can have a proximal portion which is closer to the aneurysm neck, a distal portion which is farther from the aneurysm neck, and an intermediate portion (e.g. a circumferential or equatorial band) between the proximal and distal portions, wherein the intermediate portion is more flexible, more elastic, less dense, thinner, and/or weaker than the proximal and distal portions, and wherein the intermediate portion can be selectively weakened by the application of electrical energy to facilitate movement (e.g. distortion, folding, and/or inversion) of the distal portion toward the proximal portion.

FIG. 1 shows a cross-sectional view of an aneurysm occlusion device comprising: a resilient wider-than-neck portion 104 (neck bridge) of a device transported to an aneurysm sac 101; a flexible sac-filling portion 102; and embolic members 103. FIG. 1 shows this device after it has been fully deployed within an aneurysm sac. In an example, a resilient wider-than-neck portion of a device can be expanded into a bowl shape in a multi-step transition from its first (constrained) configuration to its second (expanded) configuration. In an example of a multi-step transition, the resilient wider-than-neck portion can be expanded to a spherical or ellipsoidal shape in a first step and then this sphere or ellipsoid can be collapsed into a (two-layer) bowl shape in a second step. In an example, it can be collapsed from a spherical or ellipsoidal shape to a bowl shape by pulling a wire, cord, string, or cable which is connected to its distal surface but not connected to its proximal surface. Relevant example variations discussed elsewhere in this disclosure or in priority-linked disclosures can also be applied to this example.

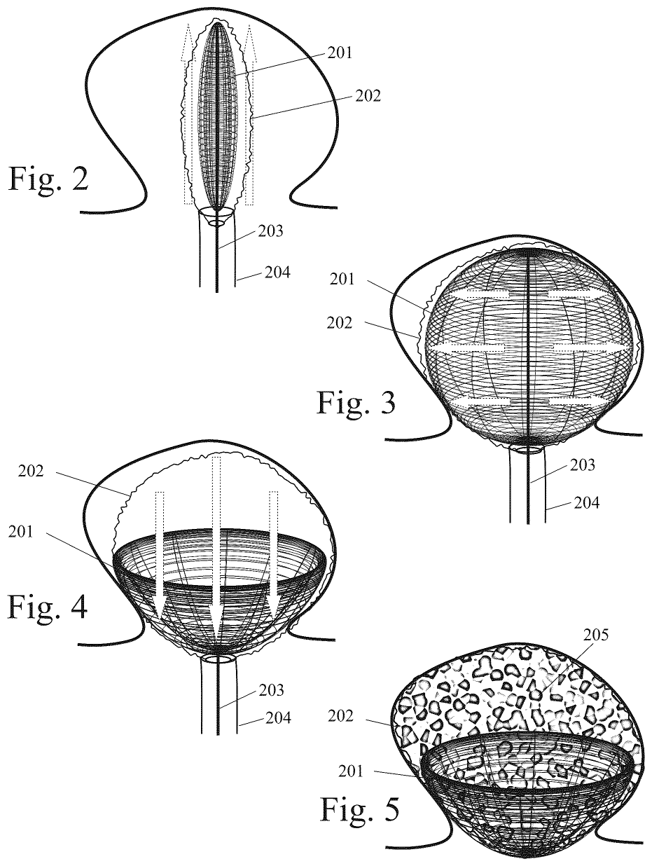

FIGS. 2 through 5 show four sequential views of an aneurysm occlusion device with a spherical stent which is collapsed into a hemisphere in order to create a double-layer barrier near an aneurysm neck. This device comprises: a spherical stent 201 which is configured to be inserted and then collapsed into a hemisphere within an aneurysm sac; a flexible expanding member 202; and a plurality of embolic members 205. This aneurysm occlusion device also includes a wire 203 which is connected to the stent and a catheter 204 through which the device is delivered to the aneurysm sac.

FIG. 2 shows the device at a first point in time when the device is exiting a catheter into an aneurysm sac. FIG. 3 shows the device at a second point in time when the cross-sectional width of the spherical stent is expanding (and the stent is transitioning from its first configuration to its second configuration) within the aneurysm sac. FIG. 4 shows the device at a third point in time when the spherical stent has collapsed into a hemispherical shape (e.g. by pulling on the wire attached to its distal end). FIG. 5 shows the device at a fourth point in time after the flexible expanding member has been filled with the plurality of embolic members and now conforms to the irregular-shaped walls of the aneurysm sac and the catheter has been removed. Relevant example variations discussed elsewhere in this disclosure or in priority-linked disclosures can also be applied to this example.

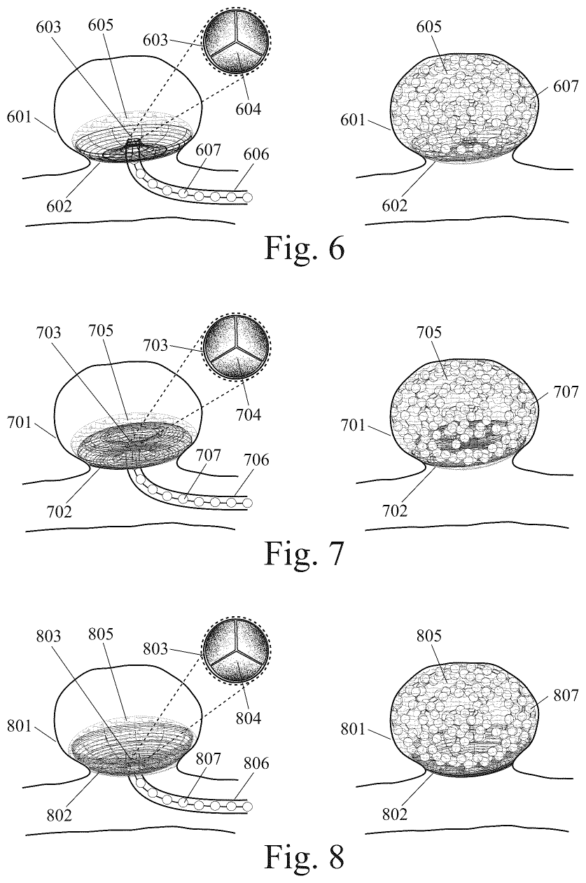

The left and right sides of FIG. 6 show two sequential views of an example of an aneurysm occlusion device comprising: a distal flexible net 605 which is inserted into an aneurysm 601; a proximal half-torus mesh 602 which is configured to be radially expanded within the aneurysm to bridge the neck of the aneurysm; a central opening 603 in the half-torus mesh; a valve 604 in the central opening; and a string-of-pearls embolic member (e.g. a longitudinal series of embolic components which are connected by a flexible filament or wire) 607 which is delivered through a catheter 606 and inserted through the valve into the distal flexible net, thereby expanding the distal flexible net to fill the sac of even an irregularly-shaped aneurysm.

The left side of FIG. 6 shows this device at a first point in time before the string-of-pearls embolic member has been inserted through the valve into the distal flexible net. The right side of FIG. 6 shows this device at a second point in time after the string-of-pearls embolic member has been inserted through the valve into the distal flexible net. In an example, the distal flexible net can be attached to the half-torus mesh. In an example, the distal flexible net can be attached to the distal surface of the half-torus mesh. In an example, the distal flexible net can be attached to the outer perimeter of the half-torus mesh. In an example, the distal flexible net can be separate from the half-torus mesh. In an example, the distal flexible mesh can be made from a polymer and the half-torus mesh can be made from metal.

In an example, a half-torus mesh can be the lower surface of the lower half of a torus. This is analogous to the lower surface of a half of a bagel lying flat on a surface. Following this analogy, the central opening in the half-torus is analogous to the hole in a half bagel, although probably not as relatively large as the hole in a half bagel. In an example, the cross-sectional area of the central opening in the half-torus mesh can be between 5% to 15% of the maximum cross-sectional area of the half-torus mesh. In an example, the cross-sectional area of the central opening in the half-torus mesh can be between 10% to 30% of the maximum cross-sectional area of the half-torus mesh. In an example, a half-torus mesh can be created geometrically by rotating an upward-opening arc (e.g. a section of a circle or a parabola) around a vertical axis (in space) which is to the right or left of the arc. In an example, the central portion of a half-torus mesh can comprise an upward-rising cone, analogous to the cone of a volcano, with the opening being where the crater of a volcano would be. In an example, the half-torus mesh can radially expand within the aneurysm sac to a width which is greater than the width of the aneurysm neck.

In an example, a half-torus mesh can have uniform porosity. In an example, a half-torus mesh can have a uniform durometer level. In an example, a half-torus mesh can have uniform elasticity. In an example, the outer perimeter of the half-torus mesh can have greater porosity than the central portion of the half-torus mesh. In an example, the outer perimeter of the half-torus mesh can have a greater durometer level than the central portion of the half-torus mesh. In an example, the outer perimeter of the half-torus mesh can be more elastic than the central portion of the half-torus mesh. In an example, the outer perimeter of the half-torus mesh can have lower porosity than the central portion of the half-torus mesh. In an example, the outer perimeter of the half-torus mesh can have a lower durometer level than the central portion of the half-torus mesh. In an example, the outer perimeter of the half-torus mesh can be less elastic than the central portion of the half-torus mesh.

In an example, a valve in a central opening can be a leaflet valve. In an example, a valve in a central opening can be a bi-leaflet valve or tri-leaflet valve, analogous to a heart valve. In an example, a valve can passively open when a string-of-pearls embolic member is pushed through it and passively close when the end of the embolic member passes or when a portion of the embolic member is detached and removed. In an example, such a valve allows a string-of-pearls embolic member to be inserted into the distal flexible net after the half-torus mesh has been expanded in the aneurysm, but closes to reduce blood flow into the aneurysm after the end of the embolic member has passed through the valve. In an alternative example, an active valve can be remotely opened and/or closed by the operator of the device. In an example, an active valve can be remotely opened and/or closed by an operator by the application of electromagnetic energy. In an example, an active valve can be remotely opened and/or closed by an operator by pulling a filament. In an example, an active valve can be remotely opened and/or closed by an operator by pushing, pulling, or rotating a wire. In an example, an active valve can be remotely opened and/or closed by an operator by cutting, pulling, or pushing a flap or plug. Relevant example variations discussed elsewhere in this disclosure or in priority-linked disclosures can also be applied to this example.

The left and right sides of FIG. 7 show two sequential views of an example of an aneurysm occlusion device comprising: a distal flexible net 705 which is inserted into an aneurysm 701; a proximal toroidal mesh 702 which is configured to be radially expanded within the aneurysm to bridge the neck of the aneurysm; a central opening 703 in the toroidal mesh; a valve 704 in the central opening; and a string-of-pearls embolic member (e.g. a longitudinal series of embolic components which are connected by a flexible filament or wire) 707 which is delivered through a catheter 706 and inserted through the valve into the distal flexible net, thereby expanding the distal flexible net to fill the sac of even an irregularly-shaped aneurysm.

The left side of FIG. 7 shows the device at a first point in time before the string-of-pearls embolic member has been inserted through the valve into the distal flexible net. The right side of FIG. 7 shows the device at a second point in time after the string-of-pearls embolic member has been inserted through the valve into the distal flexible net. In an example, the distal flexible net can be attached to the toroidal mesh. In an example, the distal flexible net can be attached to the distal surface of the toroidal mesh. In an example, the distal flexible net can be attached to the outer perimeter of the toroidal mesh. In an example, the distal flexible net can be separate from the toroidal mesh. In an example, the distal flexible mesh can be made from a polymer and the toroidal mesh can be made from metal.

In an example, a toroidal mesh can be the outer surface of a torus. This is analogous to the outer surface of a bagel. Following this analogy, the central opening in the toroidal mesh is analogous to the hole in a bagel, although probably not as relatively large as the hole in a bagel. In an example, the cross-sectional area of the central opening in the toroidal mesh can be between 5% to 15% of the maximum cross-sectional area of the toroidal mesh. In an example, the cross-sectional area of the central opening in the toroidal mesh can be between 10% to 30% of the maximum cross-sectional area of the toroidal mesh. In an example, a toroidal mesh can be created geometrically by rotating a circle or ellipse around a vertical axis (in space) which is to the right or left of the circle or ellipse. In an example, the opening can have a hyperbolic cross-section. In an example, a toroidal mesh can radially expand within the aneurysm sac to a width which is greater than the width of the aneurysm neck.

In an example, a toroidal mesh can have uniform porosity. In an example, a toroidal mesh can have a uniform durometer level. In an example, a toroidal mesh can have uniform elasticity. In an example, the outer perimeter of the toroidal mesh can have greater porosity than the central portion of the toroidal mesh. In an example, the outer perimeter of the toroidal mesh can have a greater durometer level than the central portion of the toroidal mesh. In an example, the outer perimeter of the toroidal mesh can be more elastic than the central portion of the toroidal mesh. In an example, the outer perimeter of the toroidal mesh can have lower porosity than the central portion of the toroidal mesh. In an example, the outer perimeter of the toroidal mesh can have a lower durometer level than the central portion of the toroidal mesh. In an example, the outer perimeter of the toroidal mesh can be less elastic than the central portion of the toroidal mesh. Relevant example variations discussed elsewhere in this disclosure or in priority-linked disclosures can also be applied to this example.

The left and right sides of FIG. 8 show two sequential views of an example of an aneurysm occlusion device comprising: a distal flexible net 805 which is inserted into an aneurysm 801; a proximal bowl-shaped mesh 802 which is configured to be radially expanded within the aneurysm to bridge the neck of the aneurysm; a central opening 803 in the bowl-shaped mesh; a valve 804 in the central opening; and a string-of-pearls embolic member (e.g. a longitudinal series of embolic components which are connected by a flexible filament or wire) 807 which is delivered through a catheter 806 and inserted through the valve into the distal flexible net, thereby expanding the distal flexible net to fill the sac of even an irregularly-shaped aneurysm.

The left side of FIG. 8 shows the device at a first point in time before the string-of-pearls embolic member has been inserted through the valve into the distal flexible net. The right side of FIG. 8 shows the device at a second point in time after the string-of-pearls embolic member has been inserted through the valve into the distal flexible net. In an example, the distal flexible net can be attached to the bowl-shaped mesh. In an example, the distal flexible net can be attached to the distal surface of the bowl-shaped mesh. In an example, the distal flexible net can be attached to the outer perimeter of the bowl-shaped mesh. In an example, the distal flexible net can be separate from the bowl-shaped mesh. In an example, the distal flexible mesh can be made from a polymer and the bowl-shaped mesh can be made from metal.

In an example, a bowl-shaped mesh can be a section of a sphere or ellipsoid. In an example, a bowl-shaped mesh can be hemispherical. In an example, the cross-sectional area of the central opening in the bowl-shaped mesh can be between 5% to 15% of the maximum cross-sectional area of the bowl-shaped mesh. In an example, the cross-sectional area of the central opening in the bowl-shaped mesh can be between 10% to 30% of the maximum cross-sectional area of the bowl-shaped mesh. In an example, a bowl-shaped mesh can be created geometrically by rotating a circle or ellipse around a vertical axis (in space) which is to the right or left of the circle or ellipse. In an example, a bowl-shaped mesh can radially expand within the aneurysm sac to a width which is greater than the width of the aneurysm neck.

In an example, a bowl-shaped mesh can have uniform porosity. In an example, a bowl-shaped mesh can have a uniform durometer level. In an example, a bowl-shaped mesh can have uniform elasticity. In an example, the outer perimeter of the bowl-shaped mesh can have greater porosity than the central portion of the bowl-shaped mesh. In an example, the outer perimeter of the bowl-shaped mesh can have a greater durometer level than the central portion of the bowl-shaped mesh. In an example, the outer perimeter of the bowl-shaped mesh can be more elastic than the central portion of the bowl-shaped mesh. In an example, the outer perimeter of the bowl-shaped mesh can have lower porosity than the central portion of the bowl-shaped mesh. In an example, the outer perimeter of the bowl-shaped mesh can have a lower durometer level than the central portion of the bowl-shaped mesh. In an example, the outer perimeter of the bowl-shaped mesh can be less elastic than the central portion of the bowl-shaped mesh.

In an example, a valve in a central opening can be a leaflet valve. In an example, a valve in a central opening can be a bi-leaflet valve or tri-leaflet valve, analogous to a heart valve. In an example, a valve can passively open when a string-of-pearls embolic member is pushed through it and passively close when the end of the embolic member passes or when a portion of the embolic member is detached and removed. In an example, such a valve allows a string-of-pearls embolic member to be inserted into the flexible net, but closes to reduce blood flow into the aneurysm after the end of the embolic member has passed through the valve. In an alternative example, an active valve can be remotely opened and/or closed by the operator of the device. In an example, an active valve can be remotely opened and/or closed by an operator by the application of electromagnetic energy. In an example, an active valve can be remotely opened and/or closed by an operator by pulling a filament. In an example, an active valve can be remotely opened and/or closed by an operator by pushing, pulling, or rotating a wire. In an example, an active valve can be remotely opened and/or closed by an operator by cutting, pulling, or pushing a flap or plug. Relevant example variations discussed elsewhere in this disclosure or in priority-linked disclosures can also be applied to this example.

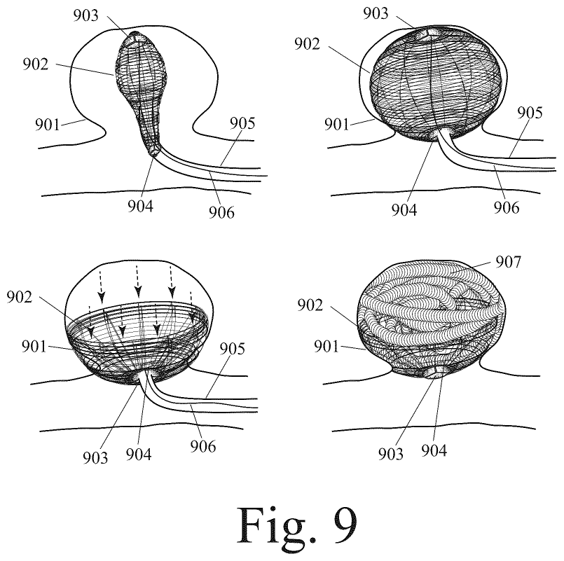

FIG. 9 shows four sequential views of an example of an aneurysm occlusion device comprising: a mesh (or framework) 902 which is configured to be inserted, radially-expanded, and then longitudinally-collapsed within an aneurysm 901 in order to bridge the aneurysm neck, wherein the mesh (or framework) has a longitudinal first configuration in which it is delivered through a catheter and inserted into an aneurysm, wherein the mesh (or framework) has a single-layer globular second configuration after it has been radially-expanded within the aneurysm, and wherein the mesh (or framework) has a double-layer bowl-shaped third configuration which bridges the aneurysm neck after distal and proximal portions of the mesh (or framework) have been longitudinally collapsed toward each other; one or more central openings (903 and 904) in the mesh (or framework); and embolic coils (or other embolic members) 906 which are delivered via a catheter 905 and inserted through the one or more openings into the aneurysm.

The upper left quadrant of FIG. 9 shows the device at a first point in time wherein the mesh (or framework) has a longitudinal first configuration and is being delivered from a catheter into an aneurysm. The upper right quadrant of FIG. 9 shows the device at a second point in time wherein the mesh (or framework) has a single-layer globular second configuration after it has been radially-expanded within the aneurysm. The lower left quadrant of FIG. 9 shows the device at a third point in time wherein the mesh (or framework) has a double-layer bowl-shaped third configuration which bridges the aneurysm neck after distal and proximal areas of the mesh (or framework) have been longitudinally collapsed toward each other. The lower right quadrant of FIG. 9 shows the device at a fourth point in time wherein embolic coils have been inserted into the aneurysm sac through one or more openings in the mesh (or framework) in its bowl-shaped configuration.

In an example, a mesh (or framework) in its bowl-shaped configuration can be a section of a sphere or ellipsoid. In an example, a mesh in its bowl-shaped configuration can be hemispherical. In an example, an opening can be between 5% to 15% of the maximum cross-sectional area of a mesh in its bowl-shaped configuration. In an example, an opening can be between 10% to 30% of the maximum cross-sectional area of a mesh in its bowl-shaped configuration. In an example, a mesh in its bowl-shaped configuration can be represented geometrically by rotating an arc of a circle or ellipse around a vertical axis (in space) which is to the right or left of the circle or ellipse. In an example, a mesh in its bowl-shaped configuration can radially expand within the aneurysm sac to a width which is greater than the width of the aneurysm neck.

In an example, a mesh can have a single layer when it is in its first and second configurations, but have a double layer when it is in its third configuration. In an example, a mesh can have more layers in its third configuration than in its first and second configurations. In an example, a string-of-pearls embolic member or a plurality of separate embolic members (e.g. microsponges or hydrogels) can be inserted into the aneurysm instead of embolic coils.

In an example, a mesh (or framework) in its bowl-shaped configuration can have uniform porosity. In an example, a mesh in its bowl-shaped configuration can have a uniform durometer level. In an example, a mesh in its bowl-shaped configuration can have uniform elasticity. In an example, the outer perimeter of a mesh in its bowl-shaped configuration can have greater porosity than the central portion of the mesh in its bowl-shaped configuration. In an example, the outer perimeter of a mesh in its bowl-shaped configuration can have a greater durometer level than the central portion of the mesh in its bowl-shaped configuration. In an example, the outer perimeter of a mesh in its bowl-shaped configuration can be more elastic than the central portion of the mesh in its bowl-shaped configuration. In an example, the outer perimeter of a mesh in its bowl-shaped configuration can have lower porosity than the central portion of the mesh in its bowl-shaped configuration. In an example, the outer perimeter of a mesh in its bowl-shaped configuration can have a lower durometer level than the central portion of the mesh in its bowl-shaped configuration. In an example, the outer perimeter of a mesh in its bowl-shaped configuration can be less elastic than the central portion of the mesh in its bowl-shaped configuration.

In an example, a mesh (or framework) can self-expand into its globular second configuration. In an example, a mesh can be longitudinally-compressed into its bowl-shaped third configuration by movement of a wire, thread, and/or filament. In an example, a mesh can be longitudinally-compressed into its bowl-shaped third configuration when a device operator pulls on a wire, thread, and/or filament. In an example, a mesh can be longitudinally-compressed into its bowl-shaped third configuration by the application of electromagnetic energy. In an example, a mesh can be longitudinally-compressed into its bowl-shaped third configuration when a device operator delivers electromagnetic energy to the mesh. In an example, a mesh can be longitudinally-compressed into its bowl-shaped third configuration by movement of a catheter. In an example, a mesh can be longitudinally-compressed into its bowl-shaped third configuration by a hydraulic or pneumatic actuator. In an example, a mesh can be longitudinally-compressed into its bowl-shaped third configuration by one or more microscale actuators (e.g. MEMS). Relevant example variations discussed elsewhere in this disclosure or in priority-linked disclosures can also be applied to this example.

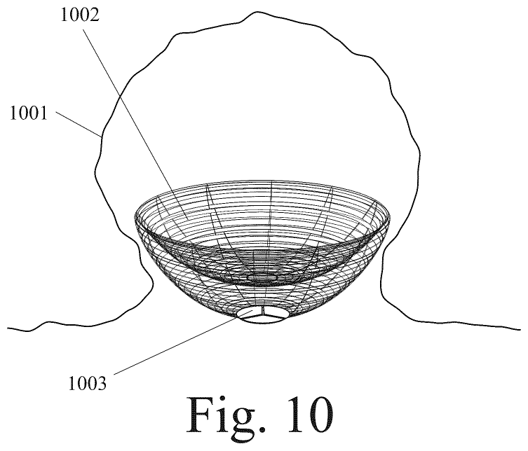

FIG. 10 shows an aneurysm occlusion device comprising: a double-layer bowl-shaped mesh 1002 which is configured to be radially-expanded to bridge the neck of an aneurysm 1001; and a valve 1003 in the double-layer bowl-shaped mesh through which embolic members (e.g. embolic coils, hydrogels, microsponges, beads, or string-of-pearls embolic strands) or liquid embolic material (which solidifies in the aneurysm) can be inserted into the aneurysm.

In an example, a bowl-shaped mesh can have uniform elasticity. In an example, a bowl-shaped mesh can have uniform porosity. In an example, a bowl-shaped mesh can have a uniform durometer level. In an example, a central portion of a bowl-shaped mesh can have a greater durometer level than a peripheral portion of a bowl-shaped mesh. In an example, the outer perimeter of a bowl-shaped mesh can have a greater durometer level than the central portion of a bowl-shaped mesh. In an example, a central portion of a bowl-shaped mesh can have a lower durometer level a peripheral portion of a bowl-shaped mesh. In an example, the outer perimeter of a bowl-shaped mesh can have a lower durometer level than the central portion of a bowl-shaped mesh. In an example, layers of a bowl-shaped mesh can be closer together near the center of the bowl-shaped mesh and farther apart around the periphery of the bowl-shaped mesh.

In an example, a central portion of a bowl-shaped mesh can have greater porosity than a peripheral portion of a bowl-shaped mesh. In an example, the outer perimeter of a bowl-shaped mesh can have greater porosity than the central portion of a bowl-shaped mesh. In an example, a central portion of a bowl-shaped mesh can have lower porosity than a peripheral portion of a bowl-shaped mesh. In an example, the outer perimeter of a bowl-shaped mesh can have lower porosity than the central portion of a bowl-shaped mesh. In an example, a central portion of a bowl-shaped mesh can be more elastic than a peripheral portion of a bowl-shaped mesh. In an example, the outer perimeter of a bowl-shaped mesh can be more elastic than the central portion of a bowl-shaped mesh. In an example, the outer perimeter of a bowl-shaped mesh can be less elastic than the central portion of a bowl-shaped mesh. In an example, layers of a bowl-shaped mesh can be farther apart near the center of the bowl-shaped mesh and closer together in the periphery of the bowl-shaped mesh.

In an example, a valve in a bowl-shaped mesh can be central to the cross-section of the bowl-shaped mesh. In an example, the cross-sectional area of a valve can be between 5% to 15% of the maximum cross-sectional area of a bowl-shaped mesh. In an example, the cross-sectional area of a valve can be between 10% to 30% of the maximum cross-sectional area of a bowl-shaped mesh. In an example, a valve can be a leaflet valve. In an example, a valve can be a bi-leaflet valve or tri-leaflet valve, analogous to a heart valve.

In an example, a valve can passively open when an embolic member is pushed through it and can passively close after the member passes through or when a portion of the member is detached. In an example, such a valve allows an embolic member to be inserted into an aneurysm, but the valve closes to reduce blood flow into the aneurysm after the embolic member has passed through the valve. In an example, an active valve can be remotely opened and/or closed by the operator of the device. In an example, an active valve can be remotely opened and/or closed by an operator by the application of electromagnetic energy. In an example, an active valve can be remotely opened and/or closed by an operator by pulling a filament. In an example, an active valve can be remotely opened and/or closed by an operator by pushing, pulling, or rotating a wire. In an example, an active valve can be remotely opened and/or closed by an operator by cutting, pulling, or pushing a flap or plug. Relevant example variations discussed elsewhere in this disclosure or in priority-linked disclosures can also be applied to this example.

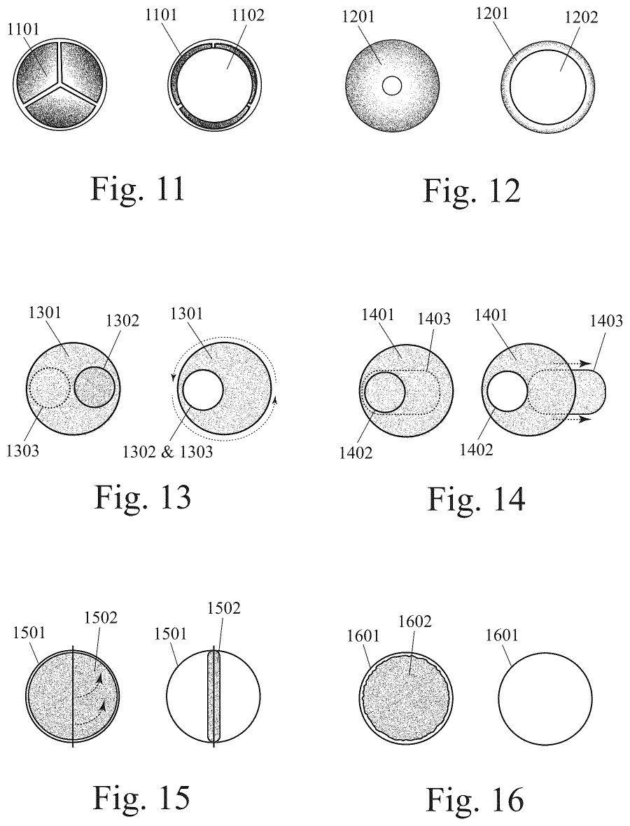

FIG. 11 shows two sequential views of an example of a leaflet valve which can be used in an aneurysm occlusion device. The left half of FIG. 11 shows this leaflet valve at a first point in time when the valve is in its closed configuration. The right half of FIG. 11 shows this leaflet valve at a second point in time when the valve is in its open configuration. Specifically, FIG. 11 shows a leaflet valve 1101 and a central opening 1102 in the valve (when it is in its open configuration).

This is just one type of valve which can be used in any of in the intrasaccular devices shown elsewhere in this disclosure or priority-linked disclosures. In an example, this leaflet valve can be positioned in an opening (or lumen) through a mesh (or net) which bridges an aneurysm neck.

When this leaflet valve is in its open configuration, embolic members (such as embolic coils, hydrogels, microsponges, beads, or string-of-pearls embolic strands) or liquid embolic material (which solidifies in the aneurysm) can be inserted through the opening in the mesh into an aneurysm. When this leaflet valve is in its closed configuration, it reduces blood flood through the opening in the mesh into the aneurysm. In other words, this leaflet valve can serve as a closure mechanism for an aneurysm occlusion device.

In an example, a leaflet valve can be a bi-leaflet valve or tri-leaflet valve, analogous to a heart valve. In an example, a leaflet valve can have a single leaflet or flap. In an example, a leaflet valve can have four or more leaflets or flaps. In an example, a leaflet valve can passively open when an embolic member (such as an embolic coil, hydrogel, microsponge, bead, or a string-of-pearls embolic strand) pushes through it. In an example, a leaflet valve can passively close when after the embolic member has passed through. In an example, a leaflet valve can be made from an elastomeric material. In an example, a leaflet valve can be made from a silicone-based polymer. In an example, a leaflet valve can be made from rigid material such as metal. In an example, a leaflet valve can be made from titanium and carbon. In an example, a leaflet valve can be remotely opened and/or closed by the operator of the device. In an example, a leaflet valve can be remotely opened and/or closed by an operator by the application of electromagnetic energy. Relevant example variations discussed elsewhere in this disclosure or in priority-linked disclosures can also be applied to this example.

FIG. 12 shows two sequential views of an example of an elastic annular valve which can be used in an aneurysm occlusion device. The left half of FIG. 12 shows this elastic annular valve at a first point in time when the valve is in its closed configuration. The right half of FIG. 12 shows this elastic annular valve at a second point in time when the valve is in its open configuration. Specifically, FIG. 12 shows a leaflet elastic annular valve 1201 and a central opening 1202 in the valve (when it is in its open configuration).

This is just one type of valve which can be used in any of in the intrasaccular devices shown elsewhere in this disclosure or priority-linked disclosures. In an example, this elastic annular valve can be positioned in an opening (or lumen) through a mesh (or net) which bridges an aneurysm neck. When this elastic annular valve is in its open configuration, embolic members (such as embolic coils, hydrogels, microsponges, beads, or string-of-pearls embolic strands) or liquid embolic material (which solidifies in the aneurysm) can be inserted through the opening in the mesh into an aneurysm. When this elastic annular valve is in its closed configuration, it reduces blood flood through the opening in the mesh into the aneurysm. In other words, this elastic annular valve can serve as a closure mechanism for an aneurysm occlusion device.

In an example, a valve can be an elastic annular valve. In an example, an elastic annular valve can passively open when an embolic member (such as an embolic coil, hydrogel, microsponge, bead, or a string-of-pearls embolic strand) pushes through it. In an example, an elastic annular valve can passively close when after the embolic member has passed through. In an example, an elastic annular valve can be made from an elastomeric material. In an example, an elastic annular valve can be made from a silicone-based polymer. Relevant example variations discussed elsewhere in this disclosure or in priority-linked disclosures can also be applied to this example.

FIG. 13 shows two sequential views of an example of a rotational valve which can be used in an aneurysm occlusion device. The left half of FIG. 13 shows this rotational valve at a first point in time wherein the valve is in its closed configuration. The right half of FIG. 13 shows this rotational valve at a second point in time wherein the valve is in its open configuration. Specifically, the rotational valve shown in FIG. 13 comprises an (outer) first layer 1301 with a first opening (or hole) 1302 and an (inner) second layer with a second opening (or hole) 1303.

When the first and second openings (holes) are not aligned, then the valve is in its closed configuration. When the first and second openings (holes) are aligned, then the valve is in its open configuration. In an example, the valve is changed from its closed configuration to its open configuration, or vice versa, by rotating (or revolving, pivoting, turning, or twisting) the first layer relative to the second layer, or vice versa. In an example, a rotational valve can comprise two or more overlapping (e.g. parallel) layers with openings (holes). When the openings (holes) of different layers are not aligned, then the valve is closed. When the opening (holes) of different layers are aligned, then the valve is open. In an example, the valve can be opened or closed by rotating one layer relative to the other layer. In an example, one or both layers can be rotated remotely by the operator of the device, enabling the operator to open or close the valve remotely.

This is just one type of valve which can be used in any of in the intrasaccular devices shown elsewhere in this disclosure or priority-linked disclosures. In an example, this rotational valve can be positioned in an opening (or lumen) through a mesh (or net) which bridges an aneurysm neck. When this rotational valve is in its open configuration, embolic members (such as embolic coils, hydrogels, microsponges, beads, or string-of-pearls embolic strands) or liquid embolic material (which solidifies in the aneurysm) can be inserted through the opening in the mesh into an aneurysm. When this rotational valve is in its closed configuration, it reduces blood flood through the opening in the mesh into the aneurysm. In other words, this rotational valve can serve as a closure mechanism for an aneurysm occlusion device. Relevant example variations discussed elsewhere in this disclosure or in priority-linked disclosures can also be applied to this example.

FIG. 14 shows two sequential views of an example of a sliding valve which can be used in an aneurysm occlusion device. The left half of FIG. 14 shows this sliding valve at a first point in time wherein the valve is in its closed configuration. The right half of FIG. 14 shows this sliding valve at a second point in time wherein the valve is in its open configuration. Specifically, the sliding valve shown in FIG. 14 comprises a layer 1401 with an opening (or hole) 1402 and a sliding flap (or lid) 1403. When the sliding flap (lid) covers the opening (hole), then the valve is in its closed configuration. When the sliding flap (lid) does not cover the opening (hole), then the valve is in its open configuration. In an example, the valve is changed from its closed configuration to its open configuration, or vice versa, by moving the sliding flap. In an example, the sliding flap can be moved remotely by the operator of the device, enabling the operator to open or close the valve remotely.

This is just one type of valve which can be used in any of in the intrasaccular devices shown elsewhere in this disclosure or priority-linked disclosures. In an example, this sliding valve can be positioned in an opening (or lumen) through a mesh (or net) which bridges an aneurysm neck. When this sliding valve is in its open configuration, embolic members (such as embolic coils, hydrogels, microsponges, beads, or string-of-pearls embolic strands) or liquid embolic material (which solidifies in the aneurysm) can be inserted through the opening in the mesh into an aneurysm. When this sliding valve is in its closed configuration, it reduces blood flood through the opening in the mesh into the aneurysm. In other words, this sliding valve can serve as a closure mechanism for an aneurysm occlusion device. Relevant example variations discussed elsewhere in this disclosure or in priority-linked disclosures can also be applied to this example.

FIG. 15 shows two sequential views of an example of a pivoting valve which can be used in an aneurysm occlusion device. The left half of FIG. 15 shows this pivoting valve at a first point in time wherein the valve is in its closed configuration. The right half of FIG. 15 shows this pivoting valve at a second point in time wherein the valve is in its open configuration. Specifically, the valve shown in FIG. 15 comprises a lumen (opening) 1501 with a pivoting flap (or plug) 1502. When the pivoting flap (plug) blocks the lumen (opening), then the valve is in its closed configuration. When the pivoting flap (lid) does not block the lumen (opening), then the valve is in its open configuration. In an example, the valve is changed from its closed configuration to its open configuration, or vice versa, by pivoting (rotating) the flap around a central axis. In the example of a square opening, a valve could be changed from its closed configuration to its open configuration, or vice versa, by pivoting (rotating) a flap around one side. In an example, the pivoting flap can be moved remotely by the operator of the device, enabling the operator to open or close the valve remotely. This type of pivoting valve is analogous to the valves which are used in circular air ducts for HVAC (heating, ventilation, and air conditioning) systems in buildings.

This is just one type of valve which can be used in any of in the intrasaccular devices shown elsewhere in this disclosure or priority-linked disclosures. In an example, this pivoting valve can be positioned in an opening (or lumen) through a mesh (or net) which bridges an aneurysm neck. When this pivoting valve is in its open configuration, embolic members (such as embolic coils, hydrogels, microsponges, beads, or string-of-pearls embolic strands) or liquid embolic material (which solidifies in the aneurysm) can be inserted through the opening in the mesh into an aneurysm. This type of pivoting valve is more appropriate for liquid embolic material than for coils, beads, or string-of-pearls strands which might get snagged on it. When this pivoting valve is in its closed configuration, it reduces blood flood through the opening in the mesh into the aneurysm. In other words, this pivoting valve can serve as a closure mechanism for an aneurysm occlusion device. Relevant example variations discussed elsewhere in this disclosure or in priority-linked disclosures can also be applied to this example.

FIG. 16 shows two sequential views of a plug mechanism which can be used in an aneurysm occlusion device. The left half of FIG. 16 shows this plug mechanism at a first point in time, in its closed configuration. The right half of FIG. 16 shows this plug mechanism at a second point in time, in its open configuration. Specifically, the plug mechanism shown in FIG. 16 comprises a lumen (opening) 1601 and a plug 1602 which is inserted into the lumen. When a plug blocks the lumen (opening), then the plug mechanism is in its closed configuration. When a plug does not block the lumen (opening), then the plug mechanism is in its open configuration. In an example, the plug mechanism is changed from its open configuration to its closed configuration by inserting a plug into the lumen (opening). In an example, a plug can be inserted remotely by the operator of the device, enabling the operator to close the plug mechanism remotely. In an example, a plug can be inserted into a lumen by using a guidewire or hydraulic pressure. In an example, a plug can be made from hydrogel.

This plug mechanism is just one type of closure mechanism which can be used in any of in the intrasaccular devices shown elsewhere in this disclosure or priority-linked disclosures. In an example, this plug mechanism can be positioned in an opening (or lumen) through a mesh (or net) which bridges an aneurysm neck. When this plug mechanism is in its open configuration, embolic members (such as embolic coils, hydrogels, microsponges, beads, or string-of-pearls embolic strands) or liquid embolic material (which solidifies in the aneurysm) can be inserted through the opening in the mesh into an aneurysm. When this plug mechanism is in its closed configuration, it reduces blood flood through the opening in the mesh into the aneurysm. Relevant example variations discussed elsewhere in this disclosure or in priority-linked disclosures can also be applied to this example.

FIG. 17 shows a cross-sectional view of an aneurysm occlusion device comprising: a bowl-shaped mesh 1702 which is configured to be inserted into and expanded within an aneurysm sac 1701 so as to span the neck of the aneurysm from inside the aneurysm sac, wherein the bowl-shaped mesh is formed by pinching each of the distal and proximal ends of a tubular mesh and then moving these ends toward each other; a distal lumen (e.g. cylinder, ring, band, tube, or torus) 1703 within which the distal end of the tubular mesh is pinched and/or bound; a proximal lumen (e.g. cylinder, ring, band, tube, or torus) 1704 within which the proximal end of the tubular mesh is pinched and/or bound; and one or more embolic members (e.g. coils, string-of-pearls embolic strands, hydrogels, microsponges, or beads) 1705 which are inserted through the proximal lumen and through the distal lumen into the aneurysm sac, wherein accumulation of the one or more embolic members in the aneurysm sac helps to occlude the aneurysm and to hold the bowl-shaped mesh against the neck of the aneurysm.

In an example, the distal lumen protrudes from the bowl-shaped mesh in a distal direction and the proximal lumen protrudes from the bowl-shaped mesh in a proximal direction. In an example, the bowl-shaped mesh is double-layered. In an example, the concavity of the bowl-shaped mesh opens in a distal direction. In an example, the distal and proximal ends of the tubular mesh can be moved toward each other and the bowl-shaped mesh can be formed before the device is deployed. In an example, the distal and proximal ends of the tubular mesh can be moved toward each other to form the bowl-shaped mesh after the device has been inserted into and expanded within the aneurysm sac. In an example, the distal end of the tubular mesh can be inverted or partially inverted. In an example, the proximal end of the tubular mesh can be inverted or partially inverted.

In an example, this device can further comprise a closure mechanism within the proximal lumen, wherein this mechanism is closed after embolic members have been inserted into the aneurysm sac. In an example, the device can further comprise a catheter through which embolic members are transported into the aneurysm sac. In an example, this catheter can extend through the proximal and distal lumens. In an example, this device can further comprise a flexible net, mesh, bag, or liner which is inserted into the aneurysm sac (before the bowl-shaped mesh) in order to contain the embolic members. In an example, such a flexible net, mesh, bad, or liner can expand to fill between 80% and 100% of the aneurysm sac as it is filled with embolic members. Relevant example variations discussed elsewhere in this disclosure or in priority-linked disclosures can also be applied to this example.

FIG. 18 shows a cross-sectional view of an aneurysm occlusion device comprising: a bowl-shaped mesh 1802 which is configured to be inserted into and expanded within an aneurysm sac 1801 so as to span the neck of the aneurysm from inside the aneurysm sac, wherein the bowl-shaped mesh is formed by pinching each of the distal and proximal ends of a tubular mesh and then moving these ends toward each other; a distal lumen (e.g. cylinder, ring, band, tube, or torus) 1803 within which the distal end of the tubular mesh is pinched and/or bound; a proximal lumen (e.g. cylinder, ring, band, tube, or torus) 1804 within which the proximal end of the tubular mesh is pinched and/or bound; and one or more embolic members (e.g. coils, string-of-pearls embolic strands, hydrogels, microsponges, or beads) 1805 which are inserted through the proximal lumen and through the distal lumen into the aneurysm sac, wherein accumulation of the one or more embolic members in the aneurysm sac helps to occlude the aneurysm and to hold the bowl-shaped mesh against the neck of the aneurysm.

In an example, the distal lumen extends inward from surface of the bowl-shaped mesh in a proximal direction and the proximal lumen extends outward from the bowl-shaped mesh in a proximal direction. In an example, the bowl-shaped mesh is double-layered. In an example, the concavity of the bowl-shaped mesh opens in a distal direction. In an example, the distal and proximal ends of the tubular mesh can be moved toward each other and the bowl-shaped mesh can be formed before the device is deployed. In an example, the distal and proximal ends of the tubular mesh can be moved toward each other to form the bowl-shaped mesh after the device has been inserted into and expanded within the aneurysm sac. In an example, the distal end of the tubular mesh can be inverted or partially inverted. In an example, the proximal end of the tubular mesh can be inverted or partially inverted. Relevant example variations discussed elsewhere in this disclosure or in priority-linked disclosures can also be applied to this example.

FIG. 19 shows a cross-sectional view of an aneurysm occlusion device comprising: a bowl-shaped mesh 1902 which is configured to be inserted into and expanded within an aneurysm sac 1901 so as to span the neck of the aneurysm from inside the aneurysm sac, wherein the bowl-shaped mesh is formed by pinching each of the distal and proximal ends of a tubular mesh and then moving these ends toward each other; a distal lumen (e.g. cylinder, ring, band, tube, or torus) 1903 within which the distal end of the tubular mesh is pinched and/or bound; a proximal lumen (e.g. cylinder, ring, band, tube, or torus) 1904 within which the proximal end of the tubular mesh is pinched and/or bound; and one or more embolic members (e.g. coils, string-of-pearls embolic strands, hydrogels, microsponges, or beads) 1905 which are inserted through the proximal lumen and through the distal lumen into the aneurysm sac, wherein accumulation of the one or more embolic members in the aneurysm sac helps to occlude the aneurysm and to hold the bowl-shaped mesh against the neck of the aneurysm.

In an example, the distal lumen extends inward from surface of the bowl-shaped mesh in a proximal direction and the proximal lumen extends inward from the surface of the bowl-shaped mesh in a distal direction. In an example, the bowl-shaped mesh is double-layered. In an example, the concavity of the bowl-shaped mesh opens in a distal direction. In an example, the distal and proximal ends of the tubular mesh can be moved toward each other and the bowl-shaped mesh can be formed before the device is deployed. In an example, the distal and proximal ends of the tubular mesh can be moved toward each other to form the bowl-shaped mesh after the device has been inserted into and expanded within the aneurysm sac. In an example, the distal end of the tubular mesh can be inverted or partially inverted. In an example, the proximal end of the tubular mesh can be inverted or partially inverted. Relevant example variations discussed elsewhere in this disclosure or in priority-linked disclosures can also be applied to this example.

FIG. 20 shows a cross-sectional view of an aneurysm occlusion device comprising: a bowl-shaped mesh 2002 which is configured to be inserted into and expanded within an aneurysm sac 2001 so as to span the neck of the aneurysm from inside the aneurysm sac, wherein the bowl-shaped mesh is formed by pinching each of the distal and proximal ends of a tubular mesh and then moving these ends toward each other; a distal lumen (e.g. cylinder, ring, band, tube, or torus) 2003 within which the distal end of the tubular mesh is pinched and/or bound; a proximal lumen (e.g. cylinder, ring, band, tube, or torus) 2004 within which the proximal end of the tubular mesh is pinched and/or bound; and one or more embolic members (e.g. coils, string-of-pearls embolic strands, hydrogels, microsponges, or beads) 2005 which are inserted through the proximal lumen and through the distal lumen into the aneurysm sac, wherein accumulation of the one or more embolic members in the aneurysm sac helps to occlude the aneurysm and to hold the bowl-shaped mesh against the neck of the aneurysm. Relevant example variations discussed elsewhere in this disclosure or in priority-linked disclosures can also be applied to this example.

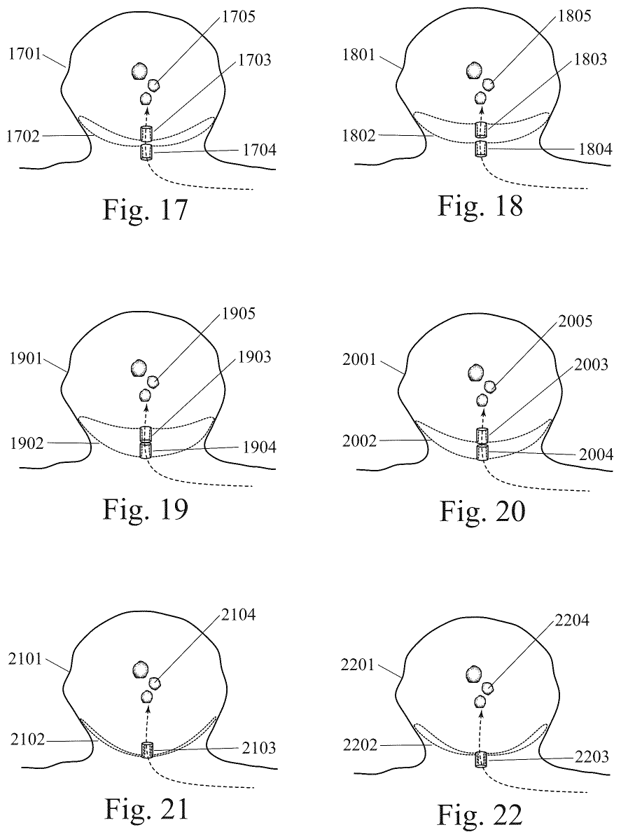

FIG. 21 shows a cross-sectional view of an aneurysm occlusion device comprising: a bowl-shaped mesh 2102 which is configured to be inserted into and expanded within an aneurysm sac 2101 so as to span the neck of the aneurysm from inside the aneurysm sac, wherein the bowl-shaped mesh is formed by pinching and/or binding together the distal and proximal ends of a tubular mesh; a lumen (e.g. cylinder, ring, band, tube, or torus) 2103 within which the distal and proximal ends of the tubular mesh are pinched and/or bound; and one or more embolic members (e.g. coils, string-of-pearls embolic strands, hydrogels, microsponges, or beads) 2104 which are inserted through the lumen into the aneurysm sac, wherein accumulation of the one or more embolic members in the aneurysm sac helps to occlude the aneurysm and to hold the bowl-shaped mesh against the neck of the aneurysm.

In an example, the lumen extends outward from surface of the bowl-shaped mesh in a distal direction. In an example, the bowl-shaped mesh is double-layered. In an example, the concavity of the bowl-shaped mesh opens in a distal direction. In an example, the distal and proximal ends of the tubular mesh can be moved and bound together and the bowl-shaped mesh can be formed before the device is deployed. In an example, the distal and proximal ends of the tubular mesh can be moved and bound together to form the bowl-shaped mesh after the device has been inserted into and expanded within the aneurysm sac. In an example, the distal end of the tubular mesh can be inverted or partially inverted. In an example, the proximal end of the tubular mesh can be inverted or partially inverted.

In an example, this device can further comprise a closure mechanism within the lumen, wherein this mechanism is closed after embolic members have been inserted into the aneurysm sac. In an example, the device can further comprise a catheter through which embolic members are transported into the aneurysm sac. In an example, this catheter can extend through the lumen. In an example, this device can further comprise a flexible net, mesh, bag, or liner which is inserted into the aneurysm sac (before the bowl-shaped mesh) in order to contain the embolic members. In an example, such a flexible net, mesh, bad, or liner can expand to fill between 80% and 100% of the aneurysm sac as it is filled with embolic members. Relevant example variations discussed elsewhere in this disclosure or in priority-linked disclosures can also be applied to this example.

FIG. 22 shows a cross-sectional view of an aneurysm occlusion device comprising: a bowl-shaped mesh 2202 which is configured to be inserted into and expanded within an aneurysm sac 2201 so as to span the neck of the aneurysm from inside the aneurysm sac, wherein the bowl-shaped mesh is formed by pinching and/or binding together the distal and proximal ends of a tubular mesh; a lumen (e.g. cylinder, ring, band, tube, or torus) 2203 within which the distal and proximal ends of the tubular mesh are pinched and/or bound; and one or more embolic members (e.g. coils, string-of-pearls embolic strands, hydrogels, microsponges, or beads) 2204 which are inserted through the lumen into the aneurysm sac, wherein accumulation of the one or more embolic members in the aneurysm sac helps to occlude the aneurysm and to hold the bowl-shaped mesh against the neck of the aneurysm. Relevant example variations discussed elsewhere in this disclosure or in priority-linked disclosures can also be applied to this example.

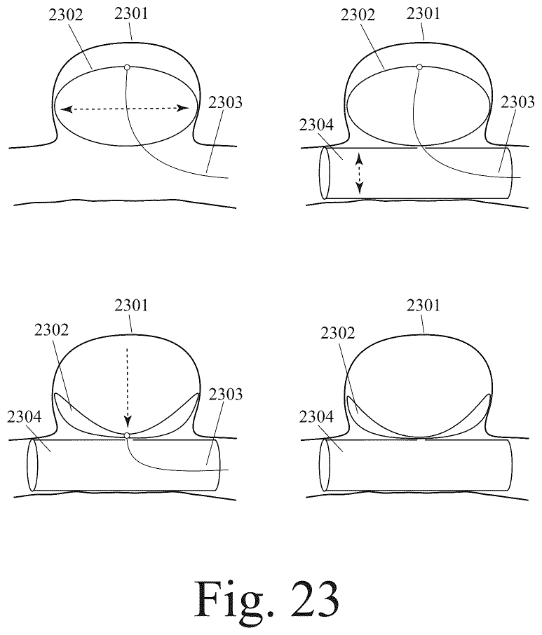

FIG. 23 shows four sequential views of an aneurysm occlusion device comprising: a globular (e.g. spherical or ellipsoidal) mesh 2302 which is configured to be radially expanded in an aneurysm 2301; a wire (or filament, string, or thread) 2303 which is attached to a distal portion of the globular mesh; and a stent 2304 which is configured to be radially expanded in a parent vessel of the aneurysm, wherein the distal portion of the globular mesh is collapsed toward the proximal portion of the globular mesh when the wire is pulled. In a variation on this wording, FIG. 23 also shows an aneurysm occlusion device comprising: a globular (e.g. spherical or ellipsoidal) mesh which is configured to be radially expanded in an aneurysm; a wire (or filament, string, or thread) which is attached to a distal portion of the globular mesh; and a stent which is configured to be radially expanded in a parent vessel of the aneurysm, wherein the distal portion of the globular mesh is collapsed toward the stent when the wire is pulled.

The upper left quadrant of FIG. 23 shows this device at a first point in time wherein the globular mesh is being radially expanded within an aneurysm. The upper right quadrant of FIG. 23 shows this device at a second point in time wherein the stent is being radially expanded within the parent vessel of the aneurysm. The lower left quadrant of FIG. 23 shows this device at a third point in time wherein the wire is pulling the distal portion of the globular mesh toward the proximal portion of the globular mesh (and also toward the stent). In an example, the globular mesh can have a first (pre-collapse) configuration which is generally spherical or ellipsoidal and a second (post-collapse) configuration which is generally hemispherical and/or bowl-shaped. The lower right quadrant of FIG. 23 shows this device at a fourth point in time after the wire has been detached and removed.

In an example, embolic material can be inserted into an aneurysm sac through an opening or lumen in a neck bridge when a closure mechanism is open and prevented from escaping out from the aneurysm sac when the closure mechanism is closed. In an example, the closure mechanism can be remotely controlled by the operating of the device. In an example, embolic material can be selected from the group consisting of: embolic coils, embolic ribbons, microsponges, hydrogels, microspheres, embolic polygons, foam, gel, congealing liquid, and string-of-pearls embolic strands. In an example, embolic coils can be inserted into an aneurysm sac through an opening or lumen in a neck bridge when a closure mechanism is open. In an example, embolic ribbons can be inserted into an aneurysm sac through an opening or lumen in a neck bridge when a closure mechanism is open. In an example, microsponges can be inserted into an aneurysm sac through an opening or lumen in a neck bridge when a closure mechanism is open.

In an example, a mesh (or framework) in its bowl-shaped configuration can be a section of a sphere or ellipsoid. In an example, a mesh in its bowl-shaped configuration can be hemispherical. In an example, an opening can be between 5% to 15% of the maximum cross-sectional area of a mesh in its bowl-shaped configuration. In an example, an opening can be between 10% to 30% of the maximum cross-sectional area of a mesh in its bowl-shaped configuration. In an example, a mesh in its bowl-shaped configuration can be represented geometrically by rotating an arc of a circle or ellipse around a vertical axis (in space) which is to the right or left of the circle or ellipse. In an example, a mesh in its bowl-shaped configuration can radially expand within the aneurysm sac to a width which is greater than the width of the aneurysm neck. In an example, a mesh can have a single layer when it is in its first and second configurations, but have a double layer when it is in its third configuration. In an example, a mesh can have more layers in its third configuration than in its first and second configurations. In an example, a string-of-pearls embolic member or a plurality of separate embolic members (e.g. microsponges or hydrogels) can be inserted into the aneurysm instead of embolic coils.

In an example, a mesh (or framework) in its bowl-shaped configuration can have uniform porosity. In an example, a mesh in its bowl-shaped configuration can have a uniform durometer level. In an example, a mesh in its bowl-shaped configuration can have uniform elasticity. In an example, the outer perimeter of a mesh in its bowl-shaped configuration can have greater porosity than the central portion of the mesh in its bowl-shaped configuration. In an example, the outer perimeter of a mesh in its bowl-shaped configuration can have a greater durometer level than the central portion of the mesh in its bowl-shaped configuration. In an example, the outer perimeter of a mesh in its bowl-shaped configuration can be more elastic than the central portion of the mesh in its bowl-shaped configuration. In an example, the outer perimeter of a mesh in its bowl-shaped configuration can have lower porosity than the central portion of the mesh in its bowl-shaped configuration. In an example, the outer perimeter of a mesh in its bowl-shaped configuration can have a lower durometer level than the central portion of the mesh in its bowl-shaped configuration. In an example, the outer perimeter of a mesh in its bowl-shaped configuration can be less elastic than the central portion of the mesh in its bowl-shaped configuration. Relevant example variations discussed elsewhere in this disclosure or in priority-linked disclosures can also be applied to this example.

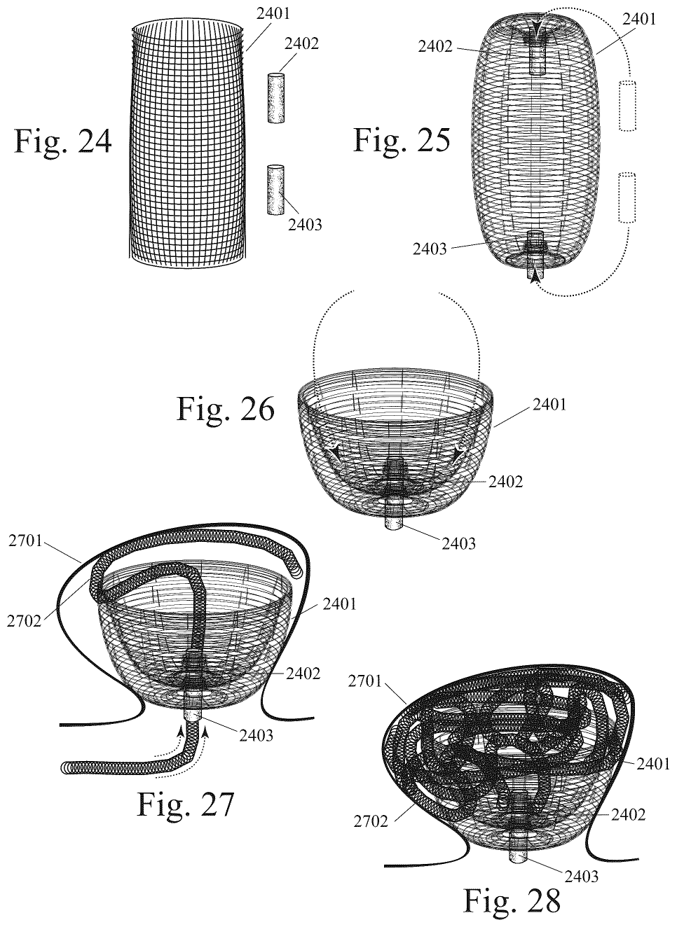

FIGS. 24 through 28 show five sequential views of another example of a device and method for aneurysm occlusion. Of these, FIGS. 24 through 26 show the device while it is being formed and FIGS. 27 and 28 show the device being deployed in an aneurysm sac. With respect to a device embodiment, FIGS. 24 through 28 show sequential views of an intrasaccular device for occluding an aneurysm comprising: a multi-layer mesh which is inserted into and expanded within an aneurysm sac, wherein the mesh has a bowl shape (e.g. hemispherical, hemi-ellipsoidal, or paraboloidal shape) with a distal-facing concavity after expansion; one or more longitudinal embolic members (e.g. embolic coils, embolic ribbons, or string-of-pearls embolic strands); and a lumen (e.g. a tube, catheter, cylinder, and/or ring) through the mesh, wherein the longitudinal embolic members are inserted through the lumen into the aneurysm sac. The device can also comprise one or more catheters, which are not shown here.

With respect to a specific components, FIGS. 24 through 28 show an intrasaccular device for occluding an aneurysm comprising: a mesh 2401 which is inserted into and expanded within an aneurysm sac 2701, wherein the mesh has a bowl shape (e.g. a hemispherical, hemi-ellipsoidal, or paraboloidal shape) with a distal-facing concavity after expansion; one or more longitudinal embolic members 2702 (e.g. embolic coils, embolic ribbons, or string-of-pearls embolic strands); a proximal lumen 2403 (e.g. a tube, catheter, cylinder, and/or ring) through the mesh, wherein the longitudinal embolic members are inserted through the lumen into the aneurysm sac; and a hub 2402 to which ends of the mesh are connected. In an example, the hub is located at the distal end of the tubular mesh until it is moved in a proximal direction during eversion of the distal end into the proximal portion of the mesh.

With respect to a method embodiment, FIGS. 24 through 28 show views of a method for occluding an aneurysm comprising: (a) connecting a lumen 2403 and a hub 2402 to the proximal and distal ends, respectively, of a tubular mesh 2401, as shown in FIG. 25; (b) everting (sometimes colloquially called inverting) the distal end of the tubular mesh into the proximal portion of the mesh in order to transform the mesh into a double-layer bowl-shaped mesh, as shown in FIG. 26; (c) radially-compressing the bowl-shaped mesh for delivery through a catheter (not shown) into an aneurysm sac 2701; (d) expanding the bowl-shaped mesh within the aneurysm sac; and then (e) inserting one or more longitudinal members 2702 through the lumen into the aneurysm sac, as shown in FIGS. 27 and 28.

In an example, a mesh can be a wire mesh. In an example, a mesh can be a polymer mesh. In an example, a mesh can comprise both wire and polymer components. In an example, a mesh can comprise a wire mesh which is (partially) covered by a low-porosity polymer membrane. In an example, a mesh can be a braided or woven mesh. In an example, a mesh can be made with shape memory material. In an example, a lumen can be a metal or polymer cylinder, ring, or tube. In an example, a lumen can remain attached to a mesh and be left in a person's body after deployment of the device. In an alternative example, a lumen can be detached from a mesh and removed from the person's body after deployment of the device.

In an example, a portion of a lumen can protrude proximally from the proximal surface of a mesh in its bowl-shaped configuration. This can help to guide a catheter into (and through) the lumen and/or to keep a catheter in the lumen until the aneurysm is filled with embolic members and the catheter is withdrawn. In another example, a lumen can be flush with the proximal surface of a mesh. In an example, the device can further comprise a closure mechanism which closes the lumen after embolic members have been inserted into the aneurysm sac. In an example, the interior of the lumen can be threaded to hold the end of the threaded end of a catheter. In an example, a device user can remove a threaded catheter from a threaded lumen by rotating a portion of the catheter and/or the lumen.

In an example, a tubular mesh can be attached to a lumen by a rigid ring, an elastic ring, a strap, a clasp, a clamp, a clip, a suture, a cord, a tape, or a band. In an example, a ring can be slipped over the mesh and the lumen to bind the mesh to the outside of the lumen. In an example, a suture or cord can be tied around the mesh and the lumen to bind the mesh to the outside of the lumen. In an example, heat can be applied to the mesh to bind the mesh to the outside of the lumen. In an example, a tubular mesh can be attached to a lumen by binding, pinching, tension, adhesion, braiding, weaving, sewing, or melting. In an example, a lumen can comprise a first cylindrical object which is inside the mesh and the mesh can be attached to the lumen by a second cylindrical object which is outside the mesh, wherein the mesh is pinched and/or held between the inner and outer cylindrical objects. In an example, a lumen can comprise a first ring which is inside the mesh and the mesh can be attached to the lumen by a second ring which is outside the mesh, wherein the mesh is pinched and/or held between the inner and outer rings.

In an example, a mesh in its bowl-shaped configuration can have a hemispherical shape. In an example, a mesh in its bowl-shaped configuration can have a shape which is a section of a (prolate) sphere which is less than half of the (prolate) sphere. In an example, a mesh in its bowl-shaped configuration can have a hemi-ellipsoidal shape. In an example, a mesh in its bowl-shaped configuration can have a shape which is a section of an ellipsoid which is less than half of the ellipsoid. In an example, a mesh in its bowl-shaped configuration can have a shape which is a (lower) section of an apple, pumpkin, barrel shape which is less than half of the overall apple, pumpkin, or barrel shape. In an example, a mesh in its bowl-shaped configuration can have an inverted umbrella shape.

In an example, a mesh in a bowl-shaped configuration can have two layers (or portions): a proximal layer (or portion) which is hemispherical, hemi-ellipsoidal, or paraboloidal and a distal layer (or portion) which is hemispherical, hemi-ellipsoidal, or paraboloidal. In an example, a mesh in a bowl-shaped configuration can have two layers: a proximal layer which is hemispherical and a distal layer which is paraboloidal. In an example, a mesh in a bowl-shaped configuration can have two layers: a proximal layer which is hemispherical and a distal layer which is hemispherical. In an example, a mesh in a bowl-shaped configuration can have two layers which meet and/or connect at the lumen and/or a hub. In an example a lumen and a hub can be connected to (e.g. be attached to) each other after a distal portion of the tubular mesh is everted into a proximal portion of the tubular mesh. In an example, a lumen and a hub can fit together (e.g. concentrically) when a distal portion of the tubular mesh is everted into a proximal portion of the tubular mesh, as shown in FIG. 26. In an example, a lumen and a hub can snap, clip, hook, or adhere together.

In an example, a mesh in a bowl-shaped configuration can have two layers which meet and/or connect distally at their respective circumferences and proximally at a lumen or hub. In an example, proximal and distal layers of a two layer mesh can have equal levels of flexibility, elasticity, porosity, thickness, and/or density. In an example, a distal layer of a two layer mesh can have greater flexibility, elasticity, or porosity than a proximal layer of the mesh. In an example, a distal layer of a two layer mesh can be less thick or dense than a proximal layer of the mesh. In an example, embolic material can also be inserted between the layers of a multi-layer bowl-shaped mesh.

In an example, the device can further comprise a guidewire which extends through a lumen. In an example, a guidewire can extend through a lumen both before expansion of the mesh within the aneurysm in order to guide a catheter through the lumen to deliver embolic members into the aneurysm sac more easily. In an example, a catheter itself can extend through the lumen before expansion of the mesh within the aneurysm. In the latter case, a catheter can be detached and withdrawn from the lumen after embolic members have been inserted through it into the aneurysm sac. In an example, an end of a catheter can have threads which are threaded into threads on the inside of the lumen. In an example, this can keep the catheter attached to the lumen until the embolic members have been delivered. Then the catheter can be detached by rotation.

In an example, longitudinal embolic members inserted through the lumen can be embolic coils. In an example, embolic members inserted through the lumen can be embolic ribbons. In an example, embolic members inserted through the lumen can be string-of-pearls strands comprising a longitudinal series of separate embolic masses (e.g. the pearls) connected by a suture, filament, or wire (e.g. the string). In an example, embolic members can be strings of hydrogels or microsponges. In an example, embolic members can be pushed through the lumen into the aneurysm sac by a pusher. In an example, embolic members can be pushed through the lumen into the aneurysm sac by a flow of liquid.

In an example, a mesh can be radially-compressed for delivery through a catheter into an aneurysm sac and then radially re-expanded to its bowl-shaped configuration within the aneurysm sac. In an example, a mesh can be radially re-expanded to a width greater than the width of the aneurysm neck. In an example, the mesh can be radially re-expanded to a width equal to (or within 90% of) the greatest width of the aneurysm sac. In an example, the mesh can be held in place against the inside of the aneurysm neck (e.g. by an attached catheter) before and while longitudinal embolic members are inserted through the lumen (and, thus, through the mesh) into the aneurysm sac. In an example, a mesh in its bowl-shaped configuration can be a neck bridge. In an example, a mesh in its bowl-shaped configuration can span, cover, and/or occlude the interior of the aneurysm neck. In an example, a bowl-shaped mesh can be expanded within the proximal half of an aneurysm sac.

In an example, a first longitudinal section of tubular mesh used to make such a device can have a first level of flexibility, elasticity, porosity, thickness, and/or density and a second longitudinal section of this tubular mesh can have a second level of flexibility, elasticity, porosity, thickness, and/or density. In this manner, a proximal layer of a two-layer bowl-shaped mesh which is formed can have a different level of flexibility, elasticity, porosity, thickness, and/or density than a distal layer of a two-layer bowl-shaped mesh. In an example, a proximal layer of a two-layer bowl-shaped mesh can be less flexible than a distal layer. In an example, a proximal layer of a two-layer bowl-shaped mesh can be less elastic than a distal layer. In an example, a proximal layer of a two-layer bowl-shaped mesh can be less porous than a distal layer. In an example, a proximal layer of a two-layer bowl-shaped mesh can be thicker than a distal layer. In an example, a proximal layer of a two-layer bowl-shaped mesh can be denser than a distal layer.

The aneurysm occlusion device shown in FIGS. 24 through 28 can also be described as comprising: an aneurysm neck bridge, wherein the neck bridge further comprises a bowl-shaped inner mesh with a distal-facing concavity and a bowl-shaped outer mesh with a distal-facing concavity; a proximal lumen, hub, or opening in the neck bridge; and embolic members which are inserted through the proximal lumen, hub, or opening into the aneurysm sac. In an example, the inner and outer bowl-shaped meshes of the neck bridge can be connected to each other around the proximal lumen, hub, or opening. In an example, the inner and outer bowl-shaped meshes can be formed by everting a tubular mesh. Relevant example variations discussed elsewhere in this disclosure or in priority-linked disclosures can also be applied to this example.

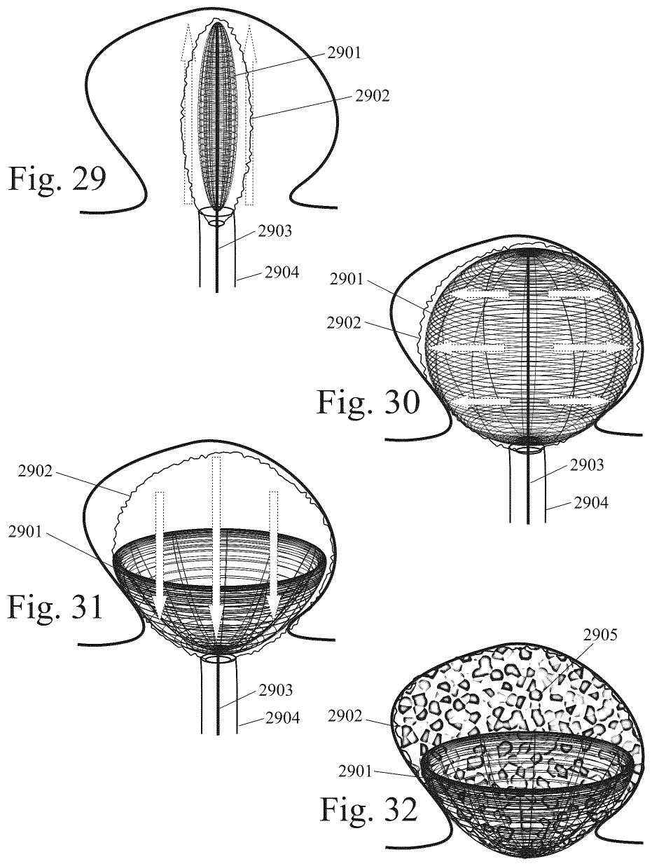

FIGS. 29 through 32 show four sequential views of an aneurysm occlusion device with a proximal stent which is compressed from a globular to a bowl-shaped configuration and embolic members/material inserted into a flexible net or mesh in a distal portion of the aneurysm sac, wherein the proximal stent is compressed by an operator pulling on a wire.

FIGS. 29 through 32 show four sequential views of an aneurysm occlusion device comprising: a proximal stent 2901, wherein the proximal stent is inserted into an aneurysm sac, expanded within the aneurysm sac into a spherical, ellipsoidal, and/or globular configuration, and then collapsed within the aneurysm sac into a hemispherical, bowl, and/or distally-concave configuration which covers the aneurysm neck; a distal flexible mesh or net 2902, wherein the flexible mesh or net is inserted into the aneurysm sac, and wherein the most distal portion of the flexible mesh or net is farther from the aneurysm neck than the most distal portion of the stent in its hemispherical, bowl, and/or distally-concave configuration; embolic members and/or material 2905, wherein the embolic members and/or material is inserted into the flexible mesh or net, wherein insertion of the embolic members and/or material into the flexible mesh or net expands the flexible mesh or net to conform to the walls of even an irregularly-shaped aneurysm sac, and wherein insertion of the embolic members and/or material into the flexible mesh or net also helps to keep the stent in place covering the aneurysm neck; a catheter and/or other lumen 2904, wherein the embolic members and/or material is delivered through the catheter and/or other lumen into the flexible mesh or net; and a wire, cord, and/or filament 2903, wherein the wire, cord, and/or filament is pulled to collapse the proximal stent into the hemispherical, bowl, and/or distally concave shape.

In an example, a flexible net or mesh can be made from material with a lower durometer than the material used to make the proximal stent. In an example, the net or mesh can be made from material with a greater elasticity than the material used to make the proximal stent. In an example, the net or mesh can be made from material which is more stretchable than the material used to make the proximal stent. In an example, the net or mesh can be made from material which is more conformable than the material used to make the proximal stent. In an example, the net or mesh can be made from material with less strength than the material used to make the proximal stent. In an example, a net or mesh can be more porous than the proximal stent. In an example, a net or mesh can be less dense than the proximal stent. In an example, a net or mesh can be more permeable to liquid than the proximal stent.

In an example, openings or holes in a flexible net or mesh can be smaller than the size (e.g. diameter, width, and/or length) of embolic members and/or material which is inserted into the net or mesh so that the embolic members and/or material do not escape out of the net or mesh. In an example, openings or holes in a flexible net or mesh can less than half of the size (e.g. diameter, width, and/or length) of embolic members and/or material which is inserted into the net or mesh so that the embolic members and/or material do not escape out of the net or mesh. In an example, openings or holes in a flexible net or mesh can have a size which is less than half of the smallest diameter and/or width of embolic members and/or material which is inserted into the net or mesh so that the embolic members and/or material do not escape out of the net or mesh. In an example, openings or holes in a flexible net or mesh can have a size which less than half of the smallest length of embolic members and/or material which is inserted into the net or mesh so that the embolic members and/or material do not escape out of the net or mesh.

In an example, embolic members and/or material inserted into the flexible net or mesh can be microspheres or microballs. In an example, embolic members and/or material inserted into the flexible net or mesh can be microsponges. In an example, embolic members and/or material inserted into the flexible net or mesh can be pieces of foam. In an example, embolic members and/or material inserted into the flexible net or mesh can be microbeads. In an example, embolic members and/or material inserted into the flexible net or mesh can be pieces of hydrogel. In an example, embolic members and/or material inserted into the flexible net or mesh can be metal embolic coils. In an example, embolic members and/or material inserted into the flexible net or mesh can be embolic ribbons. In an example, embolic members and/or material inserted into the flexible net or mesh can be yarns or filaments. In an example, embolic members and/or material can be polymer strands or coils. In an example, accumulation of embolic members and/or material in an aneurysm sac can compress a proximal stent from a spherical, ellipsoidal, and/or globular configuration to a hemispherical, bowl-shaped, and/or distally-concave configuration by pressing against the distal surface of the proximal stent.

In an example, embolic members and/or material inserted into the flexible net or mesh can be microspheres or microballs connected by a longitudinal wire, cord, and/or filament (e.g. in a string-of-pearls configuration). In an example, embolic members and/or material inserted into the flexible net or mesh can be microsponges connected by a longitudinal wire, cord, and/or filament (e.g. in a string-of-pearls configuration). In an example, embolic members and/or material inserted into the flexible net or mesh can be pieces of foam connected by a longitudinal wire, cord, and/or filament (e.g. in a string-of-pearls configuration). In an example, embolic members and/or material inserted into the flexible net or mesh can be microbeads connected by a longitudinal wire, cord, and/or filament (e.g. in a string-of-pearls configuration).

In an example, embolic members and/or material inserted into the flexible net or mesh can be pieces of hydrogel connected by a longitudinal wire, cord, and/or filament (e.g. in a string-of-pearls configuration). In an example, embolic members and/or material inserted into the flexible net or mesh can be embolic coils connected by a longitudinal wire, cord, and/or filament (e.g. in a string-of-pearls configuration). In an example, embolic members and/or material inserted into the flexible net or mesh can be embolic ribbons connected by a longitudinal wire, cord, and/or filament (e.g. in a string-of-pearls configuration). In an example, embolic members and/or material inserted into the flexible net or mesh can be yarns or filaments connected by a longitudinal wire, cord, and/or filament (e.g. in a string-of-pearls configuration).

In an example, embolic members and/or material inserted into the flexible net or mesh can be liquid which congeals and/or solidifies. In an example, embolic members and/or material inserted into the flexible net or mesh can be a polymer which congeals and/or solidifies. In an example, embolic members and/or material inserted into the flexible net or mesh can be a liquid embolic material. In an example, embolic members and/or material inserted into the flexible net or mesh can be hydrogel material. In an example, embolic members and/or material inserted into the flexible net or mesh can be congealing adhesive material. In an example, accumulation of embolic members and/or material in an aneurysm sac can compress a proximal stent from a spherical, ellipsoidal, and/or globular configuration to a hemispherical, bowl-shaped, and/or distally-concave configuration by pressing against the distal surface of the proximal stent. Relevant example variations discussed elsewhere in this disclosure or in priority-linked disclosures can also be applied to this example.

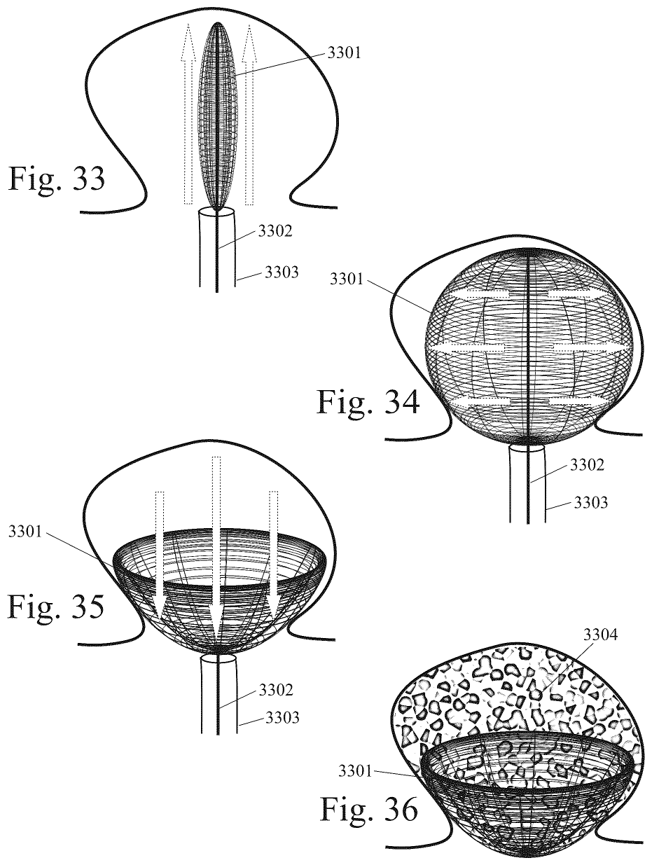

FIGS. 33 through 36 show four sequential views of an aneurysm occlusion device with a proximal stent which is compressed from a globular to a bowl-shaped configuration and embolic members/material inserted into a distal portion of the aneurysm sac, wherein the proximal stent is compressed by an operator pulling on a wire.

FIGS. 33 through 36 show four sequential views of an aneurysm occlusion device comprising: a proximal stent 3301, wherein the proximal stent is inserted into an aneurysm sac, expanded within the aneurysm sac into a spherical, ellipsoidal, and/or globular configuration, and then collapsed within the aneurysm sac into a hemispherical, bowl, and/or distally-concave configuration which covers the aneurysm neck; embolic members and/or material 3304, wherein the embolic members and/or material is inserted into a distal portion of the aneurysm sac; a catheter and/or other lumen 3303, wherein the embolic members and/or material is delivered through the catheter and/or other lumen into the distal portion of the aneurysm sac; and a wire, cord, and/or filament 3302, wherein the wire, cord, and/or filament is pulled to collapse the proximal stent into the hemispherical, bowl, and/or distally concave shape.

FIG. 33 shows this device at a first point in time when the device is exiting a catheter into an aneurysm sac. FIG. 34 shows this device at a second point in time when the cross-sectional width of the proximal stent is expanding and the stent is transitioning into its spherical, ellipsoidal, and/or globular configuration. FIG. 35 shows this device at a third point in time when the proximal stent is collapsing into its hemispherical, bowl-shaped, and/or distally-concave configuration. FIG. 36 shows this device at a fourth point in time after the distal portion of the aneurysm sac has been filled with embolic members and/or material and the catheter has been removed.

In an example, a proximal stent can be made from metal. In an example, a proximal stent can be made from Nitinol. In an example, a proximal stent can be a flexible metal mesh. In an example, a proximal stent can be a braided metal mesh. In an example, a proximal stent can be made from shape-memory material. In an example, a proximal stent can be made from a polymer. In an example, a proximal stent can be made with both metal and polymer components. In an example, a proximal stent can have a single layer in its spherical, ellipsoidal, and/or globular configuration and two (or more) layers in its hemispherical, bowl, and/or distally-concave configuration.

In an example, a proximal stent can have a longitudinal axis which spans in a proximal-to-distal direction. Proximal can be defined as being closer to the point of entry into a person's body during delivery through the person's vasculature (in the catheter) to the aneurysm and closer to the aneurysm neck after insertion into the aneurysm sac. In an example, the longitudinal axis of a proximal stent can have a first length while the proximal stent is delivered through the person's vasculature (in the catheter), a second length after expansion into a spherical, ellipsoidal, and/or globular configuration in the aneurysm sac, and a third length after collapse into a hemispherical, bowl, and/or distally-concave configuration in the aneurysm sac. In an example, the second length can be shorter than the first length. In an example, the third length can be shorter than the second length.

In an example, the width of a proximal stent in its hemispherical, bowl, and/or distally-concave configuration can be larger than the width of the aneurysm neck. In an example, the circumference of a proximal stent in its hemispherical, bowl, and/or distally-concave configuration can be larger than the circumference of the aneurysm neck. In an example, the width of a proximal stent in its hemispherical, bowl, and/or distally-concave configuration can be at least 10% larger than the width of the aneurysm neck. In an example, the circumference of a proximal stent in its hemispherical, bowl, and/or distally-concave configuration can be at least 10% larger than the circumference of the aneurysm neck. In an example, the width of a proximal stent in its hemispherical, bowl, and/or distally-concave configuration can be at least 90% of the maximum width of the aneurysm sac (parallel to the aneurysm neck). In an example, the circumference of a proximal stent in its hemispherical, bowl, and/or distally-concave configuration can be at least 90% of the circumference of the maximum circumference of the aneurysm sac (parallel to the aneurysm neck). In an example, a proximal stent can function as a neck bridge, reducing or completely blocking blood flow from the parent vessel into the aneurysm sac.

In an example, a proximal stent can be made by binding each end of a tubular mesh. In an example, a proximal stent can be made by binding and inverting ends of a tubular mesh. In an example, bound and/or inverted ends of a proximal stent can both extend into the interior of the stent in its spherical, ellipsoidal, and/or globular configuration. In an example, a distal bound and/or inverted end of a proximal stent can extend into the interior of the stent in its spherical, ellipsoidal, and/or globular configuration and a proximal bound and/or inverted end of the proximal stent can extend outward from the stent in its spherical, ellipsoidal, and/or globular configuration. In an example, a proximal bound and/or inverted end of a proximal stent can extend into the interior of the stent in its spherical, ellipsoidal, and/or globular configuration and a distal bound and/or inverted end of the proximal stent can extend outward from the stent in its spherical, ellipsoidal, and/or globular configuration.

In an example, there can be an opening and/or lumen through a proximal stent through which embolic members and/or material is inserted into a distal portion of the aneurysm sac. In an example, this opening and/or lumen can be centrally-located with respect to the proximal surface of the proximal stent. In an example, this opening and/or lumen can be centrally-located with respect to the longitudinal axis of the proximal stent. In an example, this opening and/or lumen can be an opening and/or lumen through a hub into which proximal ends of braided wires or tubes of the stent are bound or attached. In an example, this opening and/or lumen can be off-axial with respect to the longitudinal axis of the proximal stent.

In an example, these can be a closure mechanism which closes an opening and/or lumen after embolic members and/or material has been inserted into the distal portion of the aneurysm sac. In an example, this closure mechanism can be selected from the group consisting of: valve; electric detachment mechanism; elastic ring or band; threaded mechanism; sliding cover; sliding plug; filament loop; and electromagnetic solenoid. In an example, a closure mechanism can be a leaflet valve. In an example, a closure mechanism can be a one-way valve. In an example, a valve can allow embolic members and/or material to enter a distal portion of the aneurysm sac, but not allow the embolic members and/or material to exit the aneurysm sac through the opening.

In an example, a proximal stent can self-expand into its spherical, ellipsoidal, and/or globular configuration when it is released from the catheter into the aneurysm sac. In an example, a proximal stent can be compressed from its spherical, ellipsoidal, and/or globular configuration to its hemispherical, bowl, and/or distally-concave configuration by a wire, cord, and/or filament which pulls the distal end of the stent in a proximal direction (e.g. down from the dome of the aneurysm sac toward the aneurysm neck). In an example, this wire, cord, and/or filament can be pulled remotely by the person deploying the device.

In another example, a proximal stent can be compressed from its spherical, ellipsoidal, and/or globular configuration to its hemispherical, bowl, and/or distally-concave configuration by application of electromagnetic energy to the proximal stent. In an example, this electromagnetic energy can be activated remotely by the person deploying the device. In an example, a proximal stent can be compressed from its spherical, ellipsoidal, and/or globular configuration to its hemispherical, bowl, and/or distally-concave configuration by pressure from accumulating embolic members and/or material in the distal portion of the aneurysm sac. In an example, these embolic members and/or material can be delivered into a distal portion of the aneurysm sac by the person deploying the device.

In an example, a proximal stent can have a spherical shape after having been inserted into an aneurysm sac and then be collapsed into a hemispherical shape which covers the aneurysm neck. In an example, a proximal stent can have an ellipsoidal shape after having been inserted into an aneurysm sac and then be collapsed into a half-ellipsoidal shape which covers the aneurysm neck. In an example, a proximal stent can have a globular shape after having been inserted into an aneurysm sac and then be collapsed into a paraboloidal shape which covers the aneurysm neck. In an example, a proximal stent can have a spherical, ellipsoidal, and/or globular shape after having been inserted into an aneurysm sac and then be collapsed into a shape whose proximal surface is hemispherical and/or bowl-shaped and whose distal surface is a revolution of a parabola or hemisphere.

In an example, a proximal stent can have a spherical shape after having been inserted into an aneurysm sac and then be collapsed into a hemispherical shape which covers the aneurysm neck, wherein the hemispherical shape has a central opening and/or lumen through which embolic members and/or material is inserted into the aneurysm sac. In an example, a proximal stent can have an ellipsoidal shape after having been inserted into an aneurysm sac and then be collapsed into a half-ellipsoidal shape which covers the aneurysm neck, wherein the hemispherical shape has a central opening and/or lumen through which embolic members and/or material is inserted into the aneurysm sac. In an example, a proximal stent can have a globular shape after having been inserted into an aneurysm sac and then be collapsed into a paraboloidal shape which covers the aneurysm neck, wherein the hemispherical shape has a central opening and/or lumen through which embolic members and/or material is inserted into the aneurysm sac. In an example, a proximal stent can have a spherical, ellipsoidal, and/or globular shape after having been inserted into an aneurysm sac and then be collapsed into a shape whose proximal surface is hemispherical and/or bowl-shaped and whose distal surface is a revolution of a parabola or hemisphere, wherein there are central openings and/or lumens through the proximal and distal surfaces through which embolic members and/or material is inserted into the aneurysm sac.

In an example, a proximal stent can have a spherical shape after having been inserted into an aneurysm sac and then be collapsed into a hemispherical shape which covers the aneurysm neck, wherein the hemispherical shape has an off-axis opening and/or lumen through which embolic members and/or material is inserted into the aneurysm sac. In an example, a proximal stent can have an ellipsoidal shape after having been inserted into an aneurysm sac and then be collapsed into a half-ellipsoidal shape which covers the aneurysm neck, wherein the hemispherical shape has an off-axis opening and/or lumen through which embolic members and/or material is inserted into the aneurysm sac. In an example, a proximal stent can have a globular shape after having been inserted into an aneurysm sac and then be collapsed into a paraboloidal shape which covers the aneurysm neck, wherein the hemispherical shape has an off-axis opening and/or lumen through which embolic members and/or material is inserted into the aneurysm sac. In an example, a proximal stent can have a spherical, ellipsoidal, and/or globular shape after having been inserted into an aneurysm sac and then be collapsed into a shape whose proximal surface is hemispherical and/or bowl-shaped and whose distal surface is a revolution of a parabola or hemisphere, wherein there are off-axis openings and/or lumens through the proximal and distal surfaces through which embolic members and/or material is inserted into the aneurysm sac.

In an example, a distal portion (e.g. the distal half) of a proximal stent can have a lower durometer than the proximal portion (e.g. the proximal half) of the proximal stent. In an example, a distal portion (e.g. the distal half) of a proximal stent can be more flexible than the proximal portion (e.g. the proximal half) of the proximal stent. In an example, a distal portion (e.g. the distal half) of a proximal stent can be less dense than the proximal portion (e.g. the proximal half) of the proximal stent. In an example, a distal portion (e.g. the distal half) of a proximal stent can be more porous dense than the proximal portion (e.g. the proximal half) of the proximal stent.

In an example, embolic members and/or material inserted into a distal portion of the aneurysm sac can be microspheres or microballs. In an example, embolic members and/or material inserted into a distal portion of the aneurysm sac can be microsponges. In an example, embolic members and/or material inserted into a distal portion of the aneurysm sac can be pieces of foam. In an example, embolic members and/or material inserted into a distal portion of the aneurysm sac can be microbeads. In an example, embolic members and/or material inserted into a distal portion of the aneurysm sac can be pieces of hydrogel. In an example, embolic members and/or material inserted into a distal portion of the aneurysm sac can be metal embolic coils. In an example, embolic members and/or material inserted into a distal portion of the aneurysm sac can be embolic ribbons. In an example, embolic members and/or material inserted into a distal portion of the aneurysm sac can be yarns or filaments. In an example, embolic members and/or material can be polymer strands or coils. In an example, accumulation of embolic members and/or material in an aneurysm sac can compress a proximal stent from a spherical, ellipsoidal, and/or globular configuration to a hemispherical, bowl-shaped, and/or distally-concave configuration by pressing against the distal surface of the proximal stent.

In an example, embolic members and/or material inserted into a distal portion of the aneurysm sac can be microspheres or microballs connected by a longitudinal wire, cord, and/or filament (e.g. in a string-of-pearls configuration). In an example, embolic members and/or material inserted into a distal portion of the aneurysm sac can be microsponges connected by a longitudinal wire, cord, and/or filament (e.g. in a string-of-pearls configuration). In an example, embolic members and/or material inserted into a distal portion of the aneurysm sac can be pieces of foam connected by a longitudinal wire, cord, and/or filament (e.g. in a string-of-pearls configuration). In an example, embolic members and/or material inserted into a distal portion of the aneurysm sac can be microbeads connected by a longitudinal wire, cord, and/or filament (e.g. in a string-of-pearls configuration).

In an example, embolic members and/or material inserted into a distal portion of the aneurysm sac can be pieces of hydrogel connected by a longitudinal wire, cord, and/or filament (e.g. in a string-of-pearls configuration). In an example, embolic members and/or material inserted into a distal portion of the aneurysm sac can be embolic coils connected by a longitudinal wire, cord, and/or filament (e.g. in a string-of-pearls configuration). In an example, embolic members and/or material inserted into a distal portion of the aneurysm sac can be embolic ribbons connected by a longitudinal wire, cord, and/or filament (e.g. in a string-of-pearls configuration). In an example, embolic members and/or material inserted into a distal portion of the aneurysm sac can be yarns or filaments connected by a longitudinal wire, cord, and/or filament (e.g. in a string-of-pearls configuration).

In an example, embolic members and/or material inserted into a distal portion of the aneurysm sac can be liquid which congeals and/or solidifies. In an example, embolic members and/or material inserted into a distal portion of the aneurysm sac can be a polymer which congeals and/or solidifies. In an example, embolic members and/or material inserted into a distal portion of the aneurysm sac can be a liquid embolic material. In an example, embolic members and/or material inserted into a distal portion of the aneurysm sac can be hydrogel material. In an example, embolic members and/or material inserted into a distal portion of the aneurysm sac can be congealing adhesive material. In an example, accumulation of embolic members and/or material in an aneurysm sac can compress a proximal stent from a spherical, ellipsoidal, and/or globular configuration to a hemispherical, bowl-shaped, and/or distally-concave configuration by pressing against the distal surface of the proximal stent. Relevant example variations discussed elsewhere in this disclosure or in priority-linked disclosures can also be applied to this example.

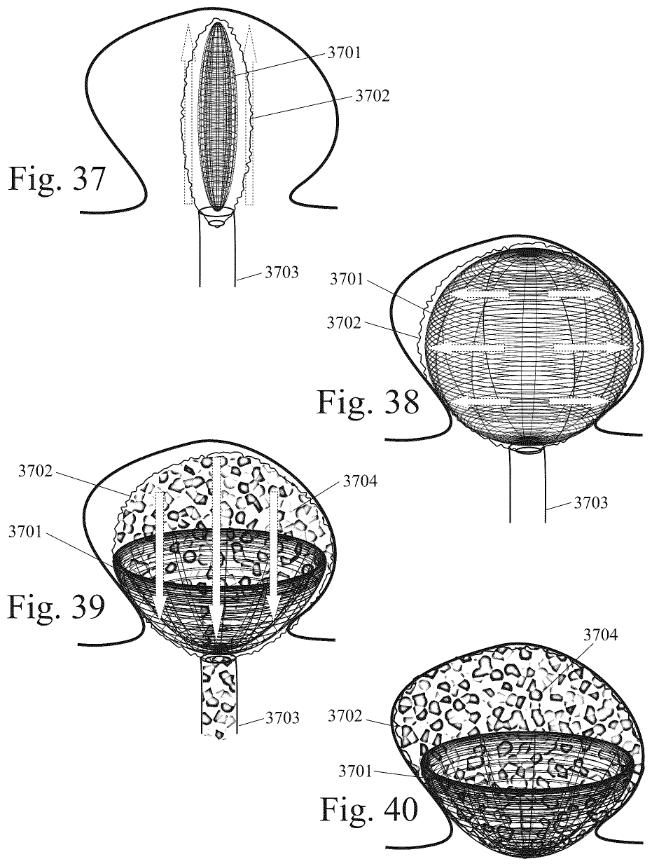

FIGS. 37 through 40 show four sequential views of an aneurysm occlusion device with a proximal stent which is compressed from a globular to a bowl-shaped configuration and embolic members/material inserted into a flexible net or mesh in a distal portion of the aneurysm sac, wherein the proximal stent is compressed by pressure from accumulating embolic members/material.

FIGS. 37 through 40 show four sequential views of an aneurysm occlusion device comprising: a proximal stent 3701, wherein the proximal stent is inserted into an aneurysm sac, expanded within the aneurysm sac into a spherical, ellipsoidal, and/or globular configuration, and then collapsed within the aneurysm sac into a hemispherical, bowl, and/or distally-concave configuration which covers the aneurysm neck; a distal flexible mesh or net 3702, wherein the flexible mesh or net is inserted into the aneurysm sac, and wherein the most distal portion of the flexible mesh or net is farther from the aneurysm neck than the most distal portion of the stent in its hemispherical, bowl, and/or distally-concave configuration; embolic members and/or material 3704, wherein the embolic members and/or material is inserted into the flexible mesh or net, wherein insertion of the embolic members and/or material into the flexible mesh or net expands the flexible mesh or net to conform to the walls of even an irregularly-shaped aneurysm sac, and wherein insertion of the embolic members and/or material into the flexible mesh or net also exerts pressure on the distal surface of the proximal stent, thereby compressing the proximal stent from its spherical, ellipsoidal, and/or globular configuration to its hemispherical, bowl, and/or distally-concave configuration; and a catheter and/or other lumen 3703, wherein the embolic members and/or material is delivered through the catheter and/or other lumen into the flexible mesh or net.

FIG. 37 shows this device at a first point in time when the device is exiting a catheter into an aneurysm sac. FIG. 38 shows this device at a second point in time when the cross-sectional width of the proximal stent is expanding and the stent is transitioning into its spherical, ellipsoidal, and/or globular configuration. FIG. 39 shows this device at a third point in time as the flexible net or mesh is being filled with embolic members and/or material and the proximal stent being collapsed into its hemispherical, bowl-shaped, and/or distally-concave configuration. FIG. 40 shows this device at a fourth point in time after the flexible net or mesh has been completely filled with embolic members and/or material (conforming to the irregular-shaped walls of the aneurysm sac) and the catheter has been removed.

In an example, there can be an opening and/or lumen through a proximal stent through which embolic members and/or material is inserted into the flexible net or mesh. In an example, this opening and/or lumen can be centrally-located with respect to the proximal surface of the proximal stent. In an example, this opening and/or lumen can be centrally-located with respect to the longitudinal axis of the proximal stent. In an example, this opening and/or lumen can be an opening and/or lumen through a hub into which proximal ends of braided wires or tubes of the stent are bound or attached. In an example, this opening and/or lumen can be off-axial with respect to the longitudinal axis of the proximal stent.

In an example, these can be a closure mechanism which closes an opening and/or lumen after embolic members and/or material has been inserted into a flexible net or mesh. In an example, this closure mechanism can be selected from the group consisting of: valve; electric detachment mechanism; elastic ring or band; threaded mechanism; sliding cover; sliding plug; filament loop; and electromagnetic solenoid. In an example, a closure mechanism can be a leaflet valve. In an example, a closure mechanism can be a one-way valve. In an example, a valve can allow embolic members and/or material to enter a flexible net or mesh through an opening, but not allow the embolic members and/or material to exit the net or mesh through the opening.

In an example, a proximal stent can self-expand into its spherical, ellipsoidal, and/or globular configuration when it is released from the catheter into the aneurysm sac. In an example, the proximal stent can be compressed from its spherical, ellipsoidal, and/or globular configuration to its hemispherical, bowl, and/or distally-concave configuration by pressure from accumulating embolic members and/or material in the flexible net or mesh. In an example, these embolic members and/or material can be delivered into the flexible net or mesh by the person deploying the device.

In another example, a proximal stent can be compressed from its spherical, ellipsoidal, and/or globular configuration to its hemispherical, bowl, and/or distally-concave configuration by a wire, cord, and/or filament which pulls the distal end of the stent in a proximal direction (e.g. down from the dome of the aneurysm sac toward the aneurysm neck). In an example, this wire, cord, and/or filament can be pulled remotely by the person deploying the device. In another example, a proximal stent can be compressed from its spherical, ellipsoidal, and/or globular configuration to its hemispherical, bowl, and/or distally-concave configuration by application of electromagnetic energy to the proximal stent. In an example, this electromagnetic energy can be activated remotely by the person deploying the device.

In an example, a proximal stent can be inside the flexible net or mesh. In an example, the flexible net or mesh can be attached to the proximal stent. In an example, an opening and/or lumen through the proximal stent can be aligned with an opening and/or lumen in the flexible net or mesh, wherein embolic members and/or material are delivered through both openings into the flexible net or mesh. In an example, a flexible net or mesh can be folded and/or compressed when it is inserted into the aneurysm sac, but expand as it is filled with embolic members and/or material. In an example, a flexible net or mesh can have radial folds as it is delivered through a catheter to an aneurysm sac. In an example, a flexible net or mesh can have longitudinal folds as it is delivered through a catheter to an aneurysm sac. In an example, a flexible net or mesh can have cross-sectional folds as it is delivered through a catheter to an aneurysm sac.

In an example, a proximal stent can have a spherical shape after having been inserted into an aneurysm sac and then be collapsed into a hemispherical shape which covers the aneurysm neck. In an example, a proximal stent can have an ellipsoidal shape after having been inserted into an aneurysm sac and then be collapsed into a half-ellipsoidal shape which covers the aneurysm neck. In an example, a proximal stent can have a globular shape after having been inserted into an aneurysm sac and then be collapsed into a paraboloidal shape which covers the aneurysm neck. In an example, a proximal stent can have a spherical, ellipsoidal, and/or globular shape after having been inserted into an aneurysm sac and then be collapsed into a shape whose proximal surface is hemispherical and/or bowl-shaped and whose distal surface is a revolution of a parabola or hemisphere.

In an example, a proximal stent can have a spherical shape after having been inserted into an aneurysm sac and then be collapsed into a hemispherical shape which covers the aneurysm neck, wherein the hemispherical shape has a central opening and/or lumen through which embolic members and/or material is inserted into the aneurysm sac. In an example, a proximal stent can have an ellipsoidal shape after having been inserted into an aneurysm sac and then be collapsed into a half-ellipsoidal shape which covers the aneurysm neck, wherein the hemispherical shape has a central opening and/or lumen through which embolic members and/or material is inserted into the aneurysm sac. In an example, a proximal stent can have a globular shape after having been inserted into an aneurysm sac and then be collapsed into a paraboloidal shape which covers the aneurysm neck, wherein the hemispherical shape has a central opening and/or lumen through which embolic members and/or material is inserted into the aneurysm sac. In an example, a proximal stent can have a spherical, ellipsoidal, and/or globular shape after having been inserted into an aneurysm sac and then be collapsed into a shape whose proximal surface is hemispherical and/or bowl-shaped and whose distal surface is a revolution of a parabola or hemisphere, wherein there are central openings and/or lumens through the proximal and distal surfaces through which embolic members and/or material is inserted into the aneurysm sac.

In an example, a proximal stent can have a spherical shape after having been inserted into an aneurysm sac and then be collapsed into a hemispherical shape which covers the aneurysm neck, wherein the hemispherical shape has an off-axis opening and/or lumen through which embolic members and/or material is inserted into the aneurysm sac. In an example, a proximal stent can have an ellipsoidal shape after having been inserted into an aneurysm sac and then be collapsed into a half-ellipsoidal shape which covers the aneurysm neck, wherein the hemispherical shape has an off-axis opening and/or lumen through which embolic members and/or material is inserted into the aneurysm sac. In an example, a proximal stent can have a globular shape after having been inserted into an aneurysm sac and then be collapsed into a paraboloidal shape which covers the aneurysm neck, wherein the hemispherical shape has an off-axis opening and/or lumen through which embolic members and/or material is inserted into the aneurysm sac. In an example, a proximal stent can have a spherical, ellipsoidal, and/or globular shape after having been inserted into an aneurysm sac and then be collapsed into a shape whose proximal surface is hemispherical and/or bowl-shaped and whose distal surface is a revolution of a parabola or hemisphere, wherein there are off-axis openings and/or lumens through the proximal and distal surfaces through which embolic members and/or material is inserted into the aneurysm sac.

In an example, a flexible net or mesh can be made from a flexible polymer. In an example, a flexible net or mesh can be made from an elastic and/or stretchable polymer. In an example, a flexible net or mesh can be elastic and/or stretchable and can expand as it is filled with embolic members and/or material. In an example, a flexible net or mesh can be sufficiently flexible to conform to the shape of even an irregularly-shaped aneurysm sac as the net or mesh is filled with embolic members and/or material. In an example, a flexible net or mesh can be sufficiently flexible to conform to the shape of even an irregularly-shaped (e.g. non-spherical) aneurysm sac as the net or mesh is filled with embolic members and/or material.

In an example, embolic members and/or material inserted into the flexible net or mesh can be microspheres or microballs. In an example, embolic members and/or material inserted into the flexible net or mesh can be microsponges. In an example, embolic members and/or material inserted into the flexible net or mesh can be pieces of foam. In an example, embolic members and/or material inserted into the flexible net or mesh can be microbeads. In an example, embolic members and/or material inserted into the flexible net or mesh can be pieces of hydrogel. In an example, embolic members and/or material inserted into the flexible net or mesh can be metal embolic coils. In an example, embolic members and/or material inserted into the flexible net or mesh can be embolic ribbons. In an example, embolic members and/or material inserted into the flexible net or mesh can be yarns or filaments. In an example, embolic members and/or material can be polymer strands or coils. In an example, accumulation of embolic members and/or material in an aneurysm sac can compress a proximal stent from a spherical, ellipsoidal, and/or globular configuration to a hemispherical, bowl-shaped, and/or distally-concave configuration by pressing against the distal surface of the proximal stent.

In an example, embolic members and/or material inserted into the flexible net or mesh can be microspheres or microballs connected by a longitudinal wire, cord, and/or filament (e.g. in a string-of-pearls configuration). In an example, embolic members and/or material inserted into the flexible net or mesh can be microsponges connected by a longitudinal wire, cord, and/or filament (e.g. in a string-of-pearls configuration). In an example, embolic members and/or material inserted into the flexible net or mesh can be pieces of foam connected by a longitudinal wire, cord, and/or filament (e.g. in a string-of-pearls configuration). In an example, embolic members and/or material inserted into the flexible net or mesh can be microbeads connected by a longitudinal wire, cord, and/or filament (e.g. in a string-of-pearls configuration).

In an example, embolic members and/or material inserted into the flexible net or mesh can be pieces of hydrogel connected by a longitudinal wire, cord, and/or filament (e.g. in a string-of-pearls configuration). In an example, embolic members and/or material inserted into the flexible net or mesh can be embolic coils connected by a longitudinal wire, cord, and/or filament (e.g. in a string-of-pearls configuration). In an example, embolic members and/or material inserted into the flexible net or mesh can be embolic ribbons connected by a longitudinal wire, cord, and/or filament (e.g. in a string-of-pearls configuration). In an example, embolic members and/or material inserted into the flexible net or mesh can be yarns or filaments connected by a longitudinal wire, cord, and/or filament (e.g. in a string-of-pearls configuration).

In an example, embolic members and/or material inserted into the flexible net or mesh can be liquid which congeals and/or solidifies. In an example, embolic members and/or material inserted into the flexible net or mesh can be a polymer which congeals and/or solidifies. In an example, embolic members and/or material inserted into the flexible net or mesh can be a liquid embolic material. In an example, embolic members and/or material inserted into the flexible net or mesh can be hydrogel material. In an example, embolic members and/or material inserted into the flexible net or mesh can be congealing adhesive material. In an example, accumulation of embolic members and/or material in an aneurysm sac can compress a proximal stent from a spherical, ellipsoidal, and/or globular configuration to a hemispherical, bowl-shaped, and/or distally-concave configuration by pressing against the distal surface of the proximal stent. Relevant example variations discussed elsewhere in this disclosure or in priority-linked disclosures can also be applied to this example.

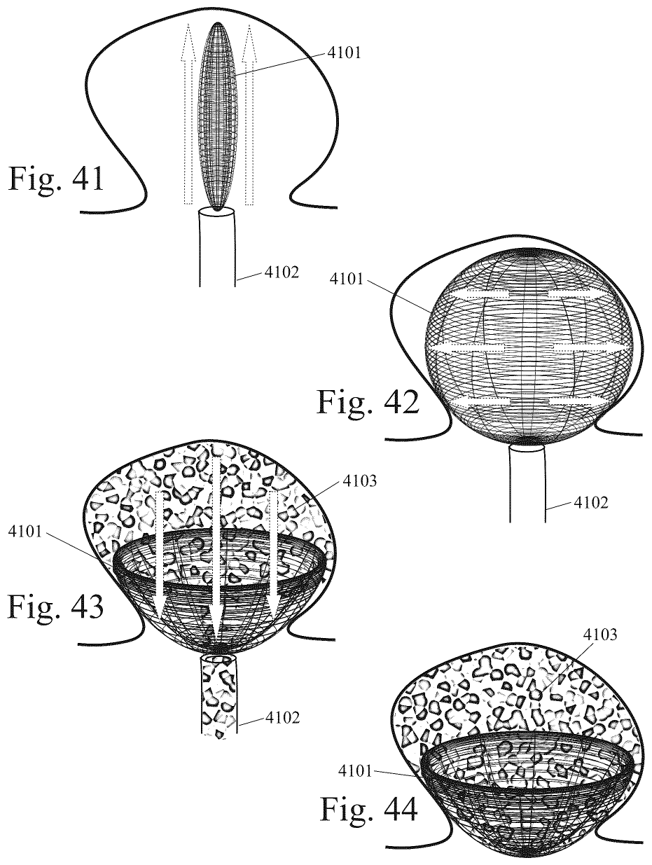

FIGS. 41 through 44 show four sequential views of an aneurysm occlusion device with a proximal stent which is compressed from a globular to a bowl-shaped configuration and embolic members/material inserted into a distal portion of the aneurysm sac, wherein the proximal stent is compressed by pressure from accumulating embolic members/material.

FIGS. 41 through 44 show four sequential views of an aneurysm occlusion device comprising: a proximal stent 4101, wherein the proximal stent is inserted into an aneurysm sac, expanded within the aneurysm sac into a spherical, ellipsoidal, and/or globular configuration, and then collapsed within the aneurysm sac into a hemispherical, bowl, and/or distally-concave configuration which covers the aneurysm neck; embolic members and/or material 4103, wherein the embolic members and/or material is inserted into a distal portion of the aneurysm sac, thereby exerting pressure on the distal surface of the proximal stent and compressing the proximal stent from its spherical, ellipsoidal, and/or globular configuration to its hemispherical, bowl, and/or distally-concave configuration; and a catheter and/or other lumen 4102, wherein the embolic members and/or material is delivered through the catheter and/or other lumen into the distal portion of the aneurysm sac.

FIG. 41 shows this device at a first point in time when the device is exiting a catheter into an aneurysm sac. FIG. 42 shows this device at a second point in time when the cross-sectional width of the proximal stent is expanding and the stent is transitioning into its spherical, ellipsoidal, and/or globular configuration. FIG. 43 shows this device at a third point in time as the distal portion of the aneurysm sac is being filled with embolic members and/or material, exerting pressure on the proximal stent which is collapsing into its hemispherical, bowl-shaped, and/or distally-concave configuration. FIG. 44 shows this device at a fourth point in time after: the distal portion of the aneurysm sac has been completely filled with embolic members and/or material; the proximal stent has been compressed into its hemispherical, bowl-shaped, and/or distally-concave configuration; and the catheter has been removed.

In an example, a proximal stent can be made from metal. In an example, a proximal stent can be made from Nitinol. In an example, a proximal stent can be a flexible metal mesh. In an example, a proximal stent can be a braided metal mesh. In an example, a proximal stent can be made from shape-memory material. In an example, a proximal stent can be made from a polymer. In an example, a proximal stent can be made with both metal and polymer components. In an example, a proximal stent can have a single layer in its spherical, ellipsoidal, and/or globular configuration and two (or more) layers in its hemispherical, bowl, and/or distally-concave configuration.

In an example, a proximal stent can have a longitudinal axis which spans in a proximal-to-distal direction. Proximal can be defined as being closer to the point of entry into a person's body during delivery through the person's vasculature (in the catheter) to the aneurysm and closer to the aneurysm neck after insertion into the aneurysm sac. In an example, the longitudinal axis of a proximal stent can have a first length while the proximal stent is delivered through the person's vasculature (in the catheter), a second length after expansion into a spherical, ellipsoidal, and/or globular configuration in the aneurysm sac, and a third length after collapse into a hemispherical, bowl, and/or distally-concave configuration in the aneurysm sac. In an example, the second length can be shorter than the first length. In an example, the third length can be shorter than the second length.

In an example, a proximal stent can be made by binding each end of a tubular mesh. In an example, a proximal stent can be made by binding and inverting ends of a tubular mesh. In an example, bound and/or inverted ends of a proximal stent can both extend into the interior of the stent in its spherical, ellipsoidal, and/or globular configuration. In an example, a distal bound and/or inverted end of a proximal stent can extend into the interior of the stent in its spherical, ellipsoidal, and/or globular configuration and a proximal bound and/or inverted end of the proximal stent can extend outward from the stent in its spherical, ellipsoidal, and/or globular configuration. In an example, a proximal bound and/or inverted end of a proximal stent can extend into the interior of the stent in its spherical, ellipsoidal, and/or globular configuration and a distal bound and/or inverted end of the proximal stent can extend outward from the stent in its spherical, ellipsoidal, and/or globular configuration.

In an example, a proximal stent can self-expand into its spherical, ellipsoidal, and/or globular configuration when it is released from the catheter into the aneurysm sac. In an example, the proximal stent is compressed from its spherical, ellipsoidal, and/or globular configuration to its hemispherical, bowl, and/or distally-concave configuration by pressure from accumulating embolic members and/or material in the distal portion of the aneurysm sac. In an example, these embolic members and/or material can be delivered into a distal portion of the aneurysm sac by the person deploying the device.

In an example, a proximal stent can have a spherical shape after having been inserted into an aneurysm sac and then be collapsed into a hemispherical shape which covers the aneurysm neck. In an example, a proximal stent can have an ellipsoidal shape after having been inserted into an aneurysm sac and then be collapsed into a half-ellipsoidal shape which covers the aneurysm neck. In an example, a proximal stent can have a globular shape after having been inserted into an aneurysm sac and then be collapsed into a paraboloidal shape which covers the aneurysm neck. In an example, a proximal stent can have a spherical, ellipsoidal, and/or globular shape after having been inserted into an aneurysm sac and then be collapsed into a shape whose proximal surface is hemispherical and/or bowl-shaped and whose distal surface is a revolution of a parabola or hemisphere.

In an example, a proximal stent can have a spherical shape after having been inserted into an aneurysm sac and then be collapsed into a hemispherical shape which covers the aneurysm neck, wherein the hemispherical shape has a central opening and/or lumen through which embolic members and/or material is inserted into the aneurysm sac. In an example, a proximal stent can have an ellipsoidal shape after having been inserted into an aneurysm sac and then be collapsed into a half-ellipsoidal shape which covers the aneurysm neck, wherein the hemispherical shape has a central opening and/or lumen through which embolic members and/or material is inserted into the aneurysm sac. In an example, a proximal stent can have a globular shape after having been inserted into an aneurysm sac and then be collapsed into a paraboloidal shape which covers the aneurysm neck, wherein the hemispherical shape has a central opening and/or lumen through which embolic members and/or material is inserted into the aneurysm sac. In an example, a proximal stent can have a spherical, ellipsoidal, and/or globular shape after having been inserted into an aneurysm sac and then be collapsed into a shape whose proximal surface is hemispherical and/or bowl-shaped and whose distal surface is a revolution of a parabola or hemisphere, wherein there are central openings and/or lumens through the proximal and distal surfaces through which embolic members and/or material is inserted into the aneurysm sac.

In an example, a proximal stent can have a spherical shape after having been inserted into an aneurysm sac and then be collapsed into a hemispherical shape which covers the aneurysm neck, wherein the hemispherical shape has an off-axis opening and/or lumen through which embolic members and/or material is inserted into the aneurysm sac. In an example, a proximal stent can have an ellipsoidal shape after having been inserted into an aneurysm sac and then be collapsed into a half-ellipsoidal shape which covers the aneurysm neck, wherein the hemispherical shape has an off-axis opening and/or lumen through which embolic members and/or material is inserted into the aneurysm sac. In an example, a proximal stent can have a globular shape after having been inserted into an aneurysm sac and then be collapsed into a paraboloidal shape which covers the aneurysm neck, wherein the hemispherical shape has an off-axis opening and/or lumen through which embolic members and/or material is inserted into the aneurysm sac. In an example, a proximal stent can have a spherical, ellipsoidal, and/or globular shape after having been inserted into an aneurysm sac and then be collapsed into a shape whose proximal surface is hemispherical and/or bowl-shaped and whose distal surface is a revolution of a parabola or hemisphere, wherein there are off-axis openings and/or lumens through the proximal and distal surfaces through which embolic members and/or material is inserted into the aneurysm sac.

In an example, embolic members and/or material inserted into a distal portion of the aneurysm sac can be microspheres or microballs. In an example, embolic members and/or material inserted into a distal portion of the aneurysm sac can be microsponges. In an example, embolic members and/or material inserted into a distal portion of the aneurysm sac can be pieces of foam. In an example, embolic members and/or material inserted into a distal portion of the aneurysm sac can be microbeads. In an example, embolic members and/or material inserted into a distal portion of the aneurysm sac can be pieces of hydrogel. In an example, embolic members and/or material inserted into a distal portion of the aneurysm sac can be metal embolic coils. In an example, embolic members and/or material inserted into a distal portion of the aneurysm sac can be embolic ribbons. In an example, embolic members and/or material inserted into a distal portion of the aneurysm sac can be yarns or filaments. In an example, embolic members and/or material can be polymer strands or coils. In an example, accumulation of embolic members and/or material in an aneurysm sac can compress a proximal stent from a spherical, ellipsoidal, and/or globular configuration to a hemispherical, bowl-shaped, and/or distally-concave configuration by pressing against the distal surface of the proximal stent.

In an example, embolic members and/or material inserted into a distal portion of the aneurysm sac can be microspheres or microballs connected by a longitudinal wire, cord, and/or filament (e.g. in a string-of-pearls configuration). In an example, embolic members and/or material inserted into a distal portion of the aneurysm sac can be microsponges connected by a longitudinal wire, cord, and/or filament (e.g. in a string-of-pearls configuration). In an example, embolic members and/or material inserted into a distal portion of the aneurysm sac can be pieces of foam connected by a longitudinal wire, cord, and/or filament (e.g. in a string-of-pearls configuration). In an example, embolic members and/or material inserted into a distal portion of the aneurysm sac can be microbeads connected by a longitudinal wire, cord, and/or filament (e.g. in a string-of-pearls configuration).

In an example, embolic members and/or material inserted into a distal portion of the aneurysm sac can be pieces of hydrogel connected by a longitudinal wire, cord, and/or filament (e.g. in a string-of-pearls configuration). In an example, embolic members and/or material inserted into a distal portion of the aneurysm sac can be embolic coils connected by a longitudinal wire, cord, and/or filament (e.g. in a string-of-pearls configuration). In an example, embolic members and/or material inserted into a distal portion of the aneurysm sac can be embolic ribbons connected by a longitudinal wire, cord, and/or filament (e.g. in a string-of-pearls configuration). In an example, embolic members and/or material inserted into a distal portion of the aneurysm sac can be yarns or filaments connected by a longitudinal wire, cord, and/or filament (e.g. in a string-of-pearls configuration).

In an example, embolic members and/or material inserted into a distal portion of the aneurysm sac can be liquid which congeals and/or solidifies. In an example, embolic members and/or material inserted into a distal portion of the aneurysm sac can be a polymer which congeals and/or solidifies. In an example, embolic members and/or material inserted into a distal portion of the aneurysm sac can be a liquid embolic material. In an example, embolic members and/or material inserted into a distal portion of the aneurysm sac can be hydrogel material. In an example, embolic members and/or material inserted into a distal portion of the aneurysm sac can be congealing adhesive material. In an example, accumulation of embolic members and/or material in an aneurysm sac can compress a proximal stent from a spherical, ellipsoidal, and/or globular configuration to a hemispherical, bowl-shaped, and/or distally-concave configuration by pressing against the distal surface of the proximal stent. Relevant example variations discussed elsewhere in this disclosure or in priority-linked disclosures can also be applied to this example.

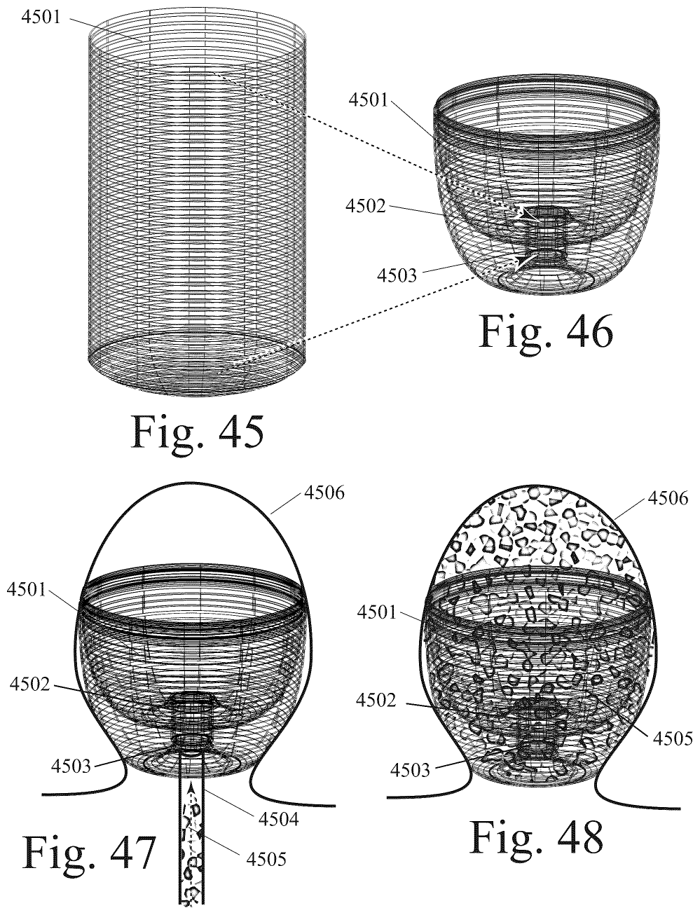

FIGS. 45 through 48 show four views, at different times, of the formation and deployment of another example of an aneurysm occlusion device comprising: at least one annular member (in this example a proximal annular member 4503 and a distal annular member 4502), wherein an annular member is selected from the group consisting of a ring, a band, a cylinder, a tube, and a catheter; a flexible net or mesh, wherein the flexible net or mesh has a spherical, ellipsoidal, generally-globular, hemispherical, and/or bowl-shaped first configuration when it is formed by encircling, pinching, inverting, and/or everting a tubular mesh 4501 at one or more longitudinal locations using the at least one annular member; wherein the flexible net or mesh has a radially-compressed second configuration for delivery through a catheter 4504 into an aneurysm sac 4506; and wherein the flexible net or mesh is inserted and expanded within the aneurysm sac; and embolic members and/or embolic material 4505 which is inserted into the interior and/or the distal-facing concavity of the flexible net or mesh through one or more of the annular members. In an example, there are two annular members: a proximal annular member which radially constrains the proximal end of the tubular mesh; and a distal annular member which radially constrains the distal end of the tubular mesh. In an example, the flexible net or mesh has a double-layer bowl shape when it is first formed from the tubular mesh.

FIG. 45 introduces tubular mesh 4501 which is used to make the intrasaccular aneurysm occlusion device. FIG. 46 shows two annular members: a proximal annular member (ring or band) 4503 which radially-constrains the proximal end of the tubular mesh; and a distal annular member (ring or band) 4502 which radially-constrains the distal end of the tubular mesh. The two annular members transform the tubular mesh into a generally-globular mesh, which, in turn, is compressed into a double-layer bowl-shaped flexible net or mesh as shown in FIG. 46. In FIG. 46, the distal portion of the tubular mesh has been compressed and inverted into the concavity of the proximal portion of the tubular mesh, thereby forming a double-layer bowl-shaped flexible net or mesh. FIG. 47 shows the flexible net or mesh after it has been radially-compressed for delivery through a catheter 4504 into an aneurysm sac 4506, wherein the flexible net or mesh has been inserted into the aneurysm sac, and wherein embolic members and/or material 4505 are starting to be delivered through the catheter (and through the annular members) into the distal-facing concavity of the bowl-shaped flexible net or mesh and the distal portion of the aneurysm sac. FIG. 48 shows the distal-facing concavity of the bowl-shaped flexible net or mesh and the distal portion of aneurysm sac having been filed with embolic members and/or material and the catheter having been removed.

In an example, an annular member is a ring or band which encircles a middle portion (between the ends) of the tubular mesh. In an example, an annular member can be a metal ring, band, or cylinder. In an example, an annular member can be a polymer ring, band, or cylinder. In an example, an annular member can be a wire, cord, or string. In an example, an annular member can be a ring or band which encircles a tubular mesh, thereby radially-constraining and/or pinching the tubular mesh but allowing embolic members and/or embolic material to pass through it into the interior and/or a concavity of the flexible net or mesh. In an example, an annular member can be a cylinder which encircles a tubular mesh, thereby radially-constraining and/or pinching the tubular mesh but allowing embolic members and/or embolic material to pass through it into the interior and/or a concavity of the flexible net or mesh. Relevant example variations discussed elsewhere in this disclosure or in priority-linked disclosures can also be applied to this example.

I claim:

1. A resilient wider-than-neck part of an aneurysm occlusion device comprising:
a mesh, net, lattice, or braid whose proximal part comprises a single layer in a first configuration and two or more overlapping layers in a second configuration.

2. The device in claim 1 wherein the resilient wider-than-neck part has a generally spherical shape which is collapsed into a generally hemispherical and/or bowl shape in an aneurysm sac in order to create a double-layer barrier across an aneurysm neck.

3. The device in claim 1 wherein there is an opening in the resilient wider-than-neck part.

4. The device in claim 3 wherein liquid embolic material is inserted through the opening into the aneurysm sac.

5. The device in claim 4 wherein a closure mechanism closes the opening after the liquid embolic material has been inserted into the aneurysm sac.

6. The device in claim 5 wherein the closure mechanism is selected from the group consisting of: tensile ring; drawstring; electromagnetic melting mechanism; insertable plug; clamp; hydrogel plug; and bioactive congealing plug.

7. The device in claim 1, wherein the device further comprises multiple delivery lumens or catheters.

8. The device in claim 7, wherein the device comprises a first delivery lumen or catheter which delivers the resilient wider-than-neck part of the device and a second delivery lumen or catheter which delivers the embolic material.

9. A resilient wider-than-neck portion of an aneurysm occlusion device comprising:
a mesh, net, or lattice which is inserted into an aneurysm sac;
wherein the mesh, net, or lattice has a first configuration in which a proximal portion of the mesh, net, or lattice has a single layer; and
wherein the mesh, net, or lattice has a second configuration in which the proximal portion of the mesh, net, or lattice has two or more layers.

10. The device in claim 9 wherein the resilient wider-than-neck portion is collapsed, compressed, and/or folded from the first configuration to the second configuration.

11. The device in claim 9 wherein the resilient wider-than-neck portion has a generally spherical or ellipsoidal shape in the first configuration.

12. The device in claim 9 wherein the resilient wider-than-neck portion has a bowl or cup shape in the second configuration.

13. The device in claim 9 wherein there is an opening in the resilient wider-than-neck portion.

14. The device in claim 13 wherein liquid embolic material is inserted through the opening into the aneurysm sac.

15. The device in claim 14 wherein a closure mechanism closes the opening after the liquid embolic material has been inserted into the aneurysm sac.

16. The device in claim 15 wherein the closure mechanism is selected from the group consisting of: tensile ring; drawstring; electromagnetic melting mechanism; insertable plug; clamp; hydrogel plug; and bioactive congealing plug.

17. The device in claim 9, wherein the device further comprises multiple delivery lumens or catheters.

18. The device in claim 17, wherein the device comprises a first delivery lumen or catheter which delivers the resilient wider-than-neck portion of the device and a second delivery lumen or catheter which delivers the embolic material.

19. An aneurysm occlusion device comprising:
a mesh, net, or lattice which is inserted into an aneurysm sac; wherein the mesh, net, or lattice is made with braided or woven filaments, wires, tubes, or strands;
wherein first ends of the filaments, wires, tubes, or strands converge at a proximal hub in the first configuration;
wherein second ends of the filaments, wires, tubes, or strands converge at a distal hub in the first configuration; and
wherein the distal hub is closer to the proximal hub in the second configuration than in the first configuration.

20. The device in claim 19 wherein the distal hub does not contact the proximal hub in the first configuration, but the distal hub does contact the proximal hub in the second configuration.
